SEQUENCE LISTING (35 sequences)

SEQ ID NO: 1 - protein, 179 aa, Homo sapiens
  MISC_FEATURE: Wild-type human IL-22 precursor
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 2 - protein, 179 aa, Artificial sequence
  Synthetic construct
  MISC_FEATURE: LH36
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMAEVVPFLAALSNALSTCHIEGDDLHIQRNVQKLKDTVKKLGESGEIKAIGELDLLFMSLRNACI

SEQ ID NO: 3 - protein, 179 aa, Artificial sequence
  Synthetic construct
  MISC_FEATURE: human 22-B3 (LH60)
MAALQKSVSSFLMGTLATSCLLLLALLVQGGAAAPISSHCRLDKENFQQPYITNRTFMLAKEASLADNNTDVRLIGEKLFHGVSMSERCYLMKQVLNFTLEEVLFPQSDRFQPYMAEVVPFLAALSNALSTCHIEGDDLHIQRNVQKLKDTVKKLGESGEIKAIGELDLLFMSLRNACI

SEQ ID NO: 4 - protein, 179 aa, Artificial sequence
  Synthetic construct
  MISC_FEATURE: human 22-B1 (LH61)
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQAVVPFLARLSNRLSTCHIEGDDLHIQRNVQKLKDTVKKLGESGEIKAIGELDLLFMSLRNACI

SEQ ID NO: 5 - protein, 179 aa, Artificial sequence
  Synthetic construct
  MISC_FEATURE: human 22-B2 (LH62)
MAALQKSVSSFLMGTLATSCLLLLALLVQGGAAAPISSHCRLDKSAFQQPYITNRTFMLAKEASLADNNTDVRLIGEKLFHGVSMSERCYLMKQVLNFTLEEVLFPQSDRFQPYMQAVVPFLARLSNRLSTCHIEGDDLHIQRNVQKLKDTVKKLGESGEIKAIGELDLLFMSLRNACI

SEQ ID NO: 6 - protein, 179 aa, Mus musculus
  MISC_FEATURE: Wild-type mouse IL-22 precursor
MAVLQKSMSFSLMGTLAASCLLLIALWAQEANALPVNTRCKLEVSNFQQPYIVNRTFMLAKEASLADNNTDVRLIGEKLFRGVSAKDQCYLMKQVLNFTLEDVLLPQSDRFQPYMQEVVPFLTKLSNQLSSCHISGDDQNIQKNVRRLKETVKKLGESGEIKAIGELDLLFMSLRNACV

SEQ ID NO: 7 - protein, 179 aa, Artificial sequence
  Synthetic construct
  MISC_FEATURE: LH10
MAVLQKSMSFSLMGTLAASCLLLIALWAQEANALPVNTRCKLEVSNFQQPYIVNRTFMLAKEASLADNNTDVRLIGEKLFRGVSAKDQCYLMKQVLNFTLEDVLLPQSDRFQPYMAEVVPFLTALSNALSSCHISGDDQNIQKNVRRLKETVKKLGESGEIKAIGELDLLFMSLRNACV

SEQ ID NO: 8 - protein, 179 aa, Artificial sequence
  Synthetic construct
  MISC_FEATURE: mouse 22-B4 (LH24)
MAVLQKSMSFSLMGTLAASCLLLIALWAQEANALPVNTRCKLEVSNFQQPYIVNRTFMLAKEASLADNNTDVRLIGEKLFRGVSAKDQCYLMKQVLNFTLEDVLLPQSDRFQPYMAEVVPFLTDLSNALSSCHISGDDQNIQKNVRRLKETVKKLGESGEIKAIGELDLLFMSLRNACV

SEQ ID NO: 9 - protein, 179 aa, Artificial sequence
  Synthetic construct
  MISC_FEATURE: LH27
MAVLQKSMSFSLMGTLAASCLLLIALWAQEANALPVNTRCKLAVSNFQQPYIVNRTFMLAKEASLADNNTDVRLIGEKLFRGVSAKDQCYLMKQVLNFTLEDVLLPQSDRFQPYMAEVVPFLTALSNALSSCHISGDDQNIQKNVRRLKETVKKLGESGEIKAIGELDLLFMSLRNACV

SEQ ID NO: 10 - protein, 179 aa, Artificial sequence
  Synthetic construct
  MISC_FEATURE: mouse 22-B5 (LH28)
MAVLQKSMSFSLMGTLAASCLLLIALWAQEANALPVNTRCKLEVSNFAQPYIVNRTFMLAKEASLADNNTDVRLIGEKLFRGVSAKDQCYLMKQVLNFTLEDVLLPQSDRFQPYMAEVVPFLTALSNALSSCHISGDDQNIQKNVRRLKETVKKLGESGEIKAIGELDLLFMSLRNACV

SEQ ID NO: 11 - protein, 179 aa, Artificial sequence
  Synthetic construct
  MISC_FEATURE: mouse 22-B3 (LH30)
MAVLQKSMSFSLMGTLAASCLLLIALWAQEANALPVNTRCKLEVENFQQPYIVNRTFMLAKEASLADNNTDVRLIGEKLFRGVSAKDQCYLMKQVLNFTLEDVLLPQSDRFQPYMAEVVPFLTALSNALSSCHISGDDQNIQKNVRRLKETVKKLGESGEIKAIGELDLLFMSLRNACV

SEQ ID NO: 12 - protein, 179 aa, Artificial sequence
  Synthetic construct
  MISC_FEATURE: LH41
MAVLQKSMSFSLMGTLAASCLLLIALWAQEANALPVNTRCKLEVSNFQQPYIVNATFMLAKEASLADNNTDVRLIGEKLFRGVSAKDQCYLMKQVLNFTLEDVLLPQSDRFQPYMQEVVPFLTKLSNQLSSCHISGDDQNIQKNVRRLKETVKKLGESGEIKAIGELDLLFMSLRNACV

SEQ ID NO: 13 - protein, 179 aa, Artificial sequence
  Synthetic construct
  MISC_FEATURE: mouse 22-B1 (LH42)
MAVLQKSMSFSLMGTLAASCLLLIALWAQEANALPVNTRCKLEVSNFQQPYIVNRTFMLAKEASLADNNTDVRLIGEKLFRGVSAKDQCYLMKQVLNFTLEDVLLPQSDRFQPYMQAVVPFLTKLSNQLSSCHISGDDQNIQKNVRRLKETVKKLGESGEIKAIGELDLLFMSLRNACV

SEQ ID NO: 14 - protein, 179 aa, Artificial sequence
  Synthetic construct
  MISC_FEATURE: mouse 22-B2 (LH48)
MAVLQKSMSFSLMGTLAASCLLLIALWAQEANALPVNTRCKLEVSAFQQPYIVNRTFMLAKEASLADNNTDVRLIGEKLFRGVSAKDQCYLMKQVLNFTLEDVLLPQSDRFQPYMQAVVPFLTKLSNQLSSCHISGDDQNIQKNVRRLKETVKKLGESGEIKAIGELDLLFMSLRNACV

SEQ ID NO: 15 - DNA, 24 bp, Artificial sequence
  Synthetic construct
  misc_feature: GAPDH qPCR primer
gtggagtcatactggaacatgtag

SEQ ID NO: 16 - DNA, 19 bp, Artificial sequence
  Synthetic construct
  misc_feature: GAPDH qPCR primer
aatggtgaaggtcggtgtg

SEQ ID NO: 17 - DNA, 22 bp, Artificial sequence
  Synthetic construct
  misc_feature: IL22R1 qPCR primer
ctcgtattctctctgtttgcct

SEQ ID NO: 18 - DNA, 22 bp, Artificial sequence
  Synthetic construct
  misc_feature: IL22R1 qPCR primer
catgacctgttctaccgcttag

SEQ ID NO: 19 - DNA, 21 bp, Artificial sequence
  Synthetic construct
  misc_feature: IL10R-beta qPCR primer
caggacggagactatgaggat

SEQ ID NO: 20 - DNA, 20 bp, Artificial sequence
  Synthetic construct
  misc_feature: IL10R-beta qPCR primer
acagaacaggagagtggagt

SEQ ID NO: 21 - DNA, 21 bp, Artificial sequence
  Synthetic construct
  misc_feature: Reg3-beta qPCR primer
tgttactccattcccatccac

SEQ ID NO: 22 - DNA, 21 bp, Artificial sequence
  Synthetic construct
  misc_feature: Reg3-beta qPCR primer
ctgaggcttcattcttgtcct

SEQ ID NO: 23 - DNA, 21 bp, Artificial sequence
  Synthetic construct
  misc_feature: Reg3-gamma qPCR primer
gattcgtctcccagttgatgt

SEQ ID NO: 24 - DNA, 18 bp, Artificial sequence
  Synthetic construct
  misc_feature: Reg3-gamma qPCR primer
ctccatgacccgacactg

SEQ ID NO: 25 - DNA, 21 bp, Artificial sequence
  Synthetic construct
  misc_feature: MUC1 qPCR primer
gactgctactgccattacctg

SEQ ID NO: 26 - DNA, 24 bp, Artificial sequence
  Synthetic construct
  misc_feature: MUC1 qPCR primer
cctaccatcctatgagtgaatacc

SEQ ID NO: 27 - DNA, 18 bp, Artificial sequence
  Synthetic construct
  misc_feature: CXCL1 qPCR primer
gtgccatcagagcagtct

SEQ ID NO: 28 - DNA, 20 bp, Artificial sequence
  Synthetic construct
  misc_feature: CXCL1 qPCR primer
ccaaaccgaagtcatagcca

SEQ ID NO: 29 - DNA, 20 bp, Artificial sequence
  Synthetic construct
  misc_feature: IL-6 qPCR primer
tccttagccactccttctgt

SEQ ID NO: 30 - DNA, 19 bp, Artificial sequence
  Synthetic construct
  misc_feature: IL-6 qPCR primer
agccagagtccttcagaga

SEQ ID NO: 31 - DNA, 20 bp, Artificial sequence
  Synthetic construct
  misc_feature: IL-1-beta qPCR primer
ctcttgttgatgtgctgctg

SEQ ID NO: 32 - DNA, 22 bp, Artificial sequence
  Synthetic construct
  misc_feature: IL-1-beta qPCR primer
gacctgttctttgaagttgacg

SEQ ID NO: 33 - protein, 13 aa, Artificial sequence
  Synthetic construct
  MISC_FEATURE: BAP tag
GLNDIFEAQKIEW

SEQ ID NO: 34 - protein, 146 aa, Homo sapiens
  MISC_FEATURE: Mature wild-type human IL-22
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 35 - protein, 146 aa, Mus musculus
  MISC_FEATURE: Mature wild-type murine IL-22
LPVNTRCKLEVSNFQQPYIVNRTFMLAKEASLADNNTDVRLIGEKLFRGVSAKDQCYLMKQVLNFTLEDVLLPQSDRFQPYMQEVVPFLTKLSNQLSSCHISGDDQNIQKNVRRLKETVKKLGESGEIKAIGELDLLFMSLRNACV

(19) United States
(12) Patent Application Publication
(10) Pub. No.: US 2023/0192794 A1
(43) Pub. Date: Jun. 22, 2023

(54) ENGINEERED INTERLEUKIN-22 POLYPEPTIDES AND USES THEREOF

(71) Applicant: The Board of Trustees of the Leland Stanford Junior University, Stanford, CA (US)

(72) Inventors: Kenan Christopher GARCIA, Palo Alto, CA (US); Robert Andrew SAXTON, Palo Alto, CA (US); Lukas Teoman HENNEBERG, Planegg (DE)

(21) Appl. No.: 17/995,985

(22) PCT Filed: Apr. 16, 2021
(86) PCT No.: PCT/US2021/027805

§ 371 Date: Oct. 11, 2022

Related U.S. Application Data
(60) Provisional application No. 63/011,922, filed on Apr. 17, 2020.

(51) Int. Cl. C07K 14/54; A61K 38/20; A61K 45/06; A61P 37/06; A61P 1/18

(52) U.S. Cl. CPC C07K 14/54; A61K 38/20; A61K 45/06; A61P 37/06; A61P 1/18

(57) ABSTRACT

The present disclosure relates generally to compositions and methods for modulating signal transduction mediated by interleukin-22 (IL-22). In particular, the disclosure provides novel IL-22 polypeptide variants with altered binding affinity to interleukin-10 receptor subunit beta (IL-10Rβ). Also provided are compositions and methods useful for producing such IL-22 polypeptide variants, as well as methods for modulating IL-22-mediated signaling, and/or for the treatment of conditions associated with the perturbation of signal transduction mediated by IL-22.

### CROSS-REFERENCE TO RELATED APPLICATIONS

This application claims the benefit of priority to U.S. Provisional Patent Application Ser. No. 63/011,922, filed on Apr. 17, 2020. The disclosure of the above-referenced application is herein expressly incorporated by reference it its entirety, including any drawings.

### STATEMENT REGARDING FEDERALLY SPONSORED R&D

This invention was made with Government support under contract AI51321 awarded by The National Institutes of Health. The Government has certain rights in the invention.

### INCORPORATION OF THE SEQUENCE LISTING

The material in the accompanying Sequence Listing is hereby incorporated by reference into this application. The accompanying Sequence Listing text file, named 078430-516001WO-Sequence Listing.txt, was created on Apr. 12, 2021 and is 31 KB.

### FIELD

The present disclosure relates generally to compositions and methods for modulating signal transduction mediated by interleukin-22 (IL-22). In particular, the disclosure provides novel IL-22 polypeptide variants with altered binding affinity to interleukin-10 receptor subunit beta (IL-10Rβ). Also provided are compositions and methods useful for producing such IL-22 polypeptide variants, as well as methods for modulating IL-22-mediated signaling, and/or for the treatment of conditions associated with the perturbation of signal transduction mediated by IL-22.

### BACKGROUND

Biopharmaceuticals or the use of pharmaceutical formulations containing therapeutic protein(s) for the treatment of health conditions and diseases is a core strategy for a number of pharmaceutical and biotechnology companies. For example, several members of the cytokine family have been reported to be effective in the treatment of cancer and play a major role in the development of cancer immunotherapy. Therefore, the cytokine family has been the focus of much clinical work and effort to improve its administration and bio-assimilation.

However, the clinical success of existing therapeutic approaches involving cytokines has been limited. Their limitations are often due to off-target toxicity and ineffectiveness of the cytokines, which is largely due to the fact that cytokines have receptors on both desired and undesired responder cells that counterbalance one another and lead to unwanted side effects. In recent years, cytokine engineering has emerged as a promising strategy with various attempts to tailor cytokines to arrive at recombinant cytokines with more desired activities and reduced toxicity.

In particular, interleukin-22 (IL-22) has been of particular clinical interest for immunotherapy due to its powerful immune-modulating effects and its ability to protect tissues from inflammation-associated damage without suppressing the immune system. Therapeutic administration of recombinant IL-22 is currently being tested in Phase I/II clinical trials for numerous auto-immune diseases and inflammatory disorders. These include ulcerative colitis, Crohn's disease, diabetic foot ulcer, and acute GvHD. IL-22 has also shown efficacy in mouse models of acute pancreatitis. However, IL-22 has also been shown to promote inflammation in some contexts, particularly through induction of inflammatory mediators in the liver, skin, and GI tract, therefore limiting its therapeutic and clinical utility.

Hence, there is a need for additional approaches to improve properties of IL-22 for its use as a therapeutic agent. In particular, there is a need for variants of IL-22 that can selectively activate certain downstream functions and actions over others, e.g., retain many beneficial properties of IL-22 but lack its known pro-inflammatory side effects, leading to improved use of these variants as anti-tumor agents or immune modulators in treating various relevant diseases, including autoimmune and inflammatory diseases.

### SUMMARY

The present disclosure relates generally to the field of immunology, and particularly to compositions and methods for modulating signal transduction pathway mediated by interleukin 22 (IL-22) in a subject in need thereof. As described in greater detail below, IL-22-mediated signaling can be modulated via biased agonism of STAT1-mediated pro-inflammatory function and/or STAT3-mediated signaling. More particularly, in some embodiments, the disclosure provides a new class of IL-22 polypeptide variants with modulated binding affinity for the natural ligands of IL-22, e.g., interleukin 10 receptor subunit beta (IL10Rβ). Some embodiments of the disclosure provide IL-22 partial agonists that results in a tissue-selective IL-22 signaling. Some embodiments of the disclosure provide IL-22 partial agonists that confer a biased IL-22 signaling, for example confer a reduction in a STAT1-mediated pro-inflammatory function while substantially retains its STAT3-mediated function. The disclosure also provides compositions and methods useful for producing such IL-22 polypeptide variants, methods for modulating IL-22-mediated signaling in a subject, as well as methods for the treatment of conditions associated with perturbations of signal transduction downstream of the IL-22 receptor.

In one aspect, provided herein are recombinant polypeptides including: (a) an amino acid sequence having at least 70% sequence identity to an interleukin 22 (IL-22) polypeptide having the amino acid sequence of SEQ ID NO: 1; and further including (b) one or more amino acid substitution at a position corresponding to an amino acid residue selected from the group consisting of X43, X49, X45, X46, X116, X124, and X128 of SEQ ID NO: 1.

Non-limiting exemplary embodiments of the disclosed recombinant polypeptides can include one or more of the following features. In some embodiments, the amino acid sequence further includes an additional amino acid substitution at a position corresponding to an amino acid residue selected from the group consisting of X48, X55, and X117 of SEQ ID NO: 1. In some embodiments, the one or more amino acid substitution reduces IL10Rβ-binding affinity of the recombinant IL-22 polypeptide compared to a reference IL-22 polypeptide lacking the one or more amino acid substitution. In some embodiments, the one or more amino acid substitution increases IL10Rβ-binding affinity of the recombinant IL-22 polypeptide compared to a reference IL-22 polypeptide lacking the one or more amino acid substitution. In some embodiments, the one or more amino acid substitution is at a position corresponding to an amino acid residue selected from the group consisting of X43, X45, X46, X48, X49, X55, and X117 of SEQ ID NO: 1. In some embodiments, the polypeptides of the disclosure further include a combination of amino acid substitutions at positions corresponding to amino acid residues X116, X124, X128 of SEQ ID NO: 1. In some embodiments, the amino acid sequence includes an amino acid substitution corresponding to amino acid residue X55 or X117 of SEQ ID NO: 1.

In some embodiments, the one or more amino acid substitution is independently selected from the group consisting of an alanine substitution, an arginine substitution, an aspartic acid substitution, a histidine substitution, a glutamic acid substitution, a lysine substitution, a serine substitution, a tryptophan substitution, and combinations of any thereof. In some embodiments, the one or more amino acid substitution is at a position corresponding to an amino acid residue selected from the group consisting of D43, S45, N46, Q49, Q116, R124, and R128 of SEQ ID NO: 1. In some embodiments, the polypeptides of the disclosure include an amino acid sequence having at least 70% sequence identity to SEQ ID NO: 1, and further include the amino acid substitutions corresponding to the following amino acid substitutions: (a) R55A; (b) E117A; (c) N46A/E117A; (d) Q116A/R124A/R128A; (e) Q116A/R124D/R128A; (f) D43A/Q116A/R124A/R128A; (g) S45E/Q116A/R124A/R128A; and (h) Q48A/Q116A/R124A/R128A.

In some embodiments, the polypeptides of the disclosure include an amino acid sequence having at least 70% sequence identity to SEQ ID NO: 1, and further include an amino acid substitution corresponding an amino acid residue selected from the group consisting of D43H, D43R, S45R, S45G, Q49S, Q49G, Q116W, Q116K, R124Y, and R128K of SEQ ID NO: 1.

In one aspect, provided herein are recombinant polypeptides including: (a) an amino acid sequence having at least 70% sequence identity to an interleukin 22 (IL-22) polypeptide having the amino acid sequence of SEQ ID NO: 6; and further including (b) one or more amino acid substitution at a position corresponding to an amino acid residue selected from the group consisting of X43, X45, X46, X49, X116, X124, and X128 of SEQ ID NO: 6.

In some embodiments, the amino acid sequence further include an additional amino acid substitution at a position corresponding to an amino acid residue selected from the group consisting of X48, X55, and X117 of SEQ ID NO: 6. In some embodiments, the one or more amino acid substitution reduces IL10Rβ-binding affinity of the recombinant IL-22 polypeptide compared to a reference IL-22 polypeptide lacking the one or more amino acid substitution. In some embodiments, the one or more amino acid substitution increases IL10Rβ-binding affinity of the recombinant IL-22 polypeptide compared to a reference IL-22 polypeptide lacking the one or more amino acid substitution. In some embodiments, the one or more amino acid substitution is at a position corresponding to an amino acid residue selected from the group consisting of X43, X45, X46, X48, X55, and X117 of SEQ ID NO: 6. In some embodiments, the nucleic acids of the disclosure include a combination of amino acid substitutions at positions corresponding to amino acid residues X116, X124, X128 of SEQ ID NO: 6. In some embodiments, the amino acid sequence includes an amino acid substitution corresponding to amino acid residue X55 or X117 of SEQ ID NO: 6.

In some embodiments, the one or more amino acid substitution is independently selected from the group consisting of an alanine substitution, an arginine substitution, an aspartic acid substitution, a histidine substitution, a glutamic acid substitution, a lysine substitution, a serine substitution, a tryptophan substitution, and combinations of any thereof. In some embodiments, the one or more amino acid substitution is at a position corresponding to an amino acid residue selected from the group consisting of E43, S45, N46, Q48, R55, Q116, E117, K124, Q128 of SEQ ID NO: 6. In some embodiments, the nucleic acids of the disclosure include an amino acid sequence having at least 70% sequence identity to SEQ ID NO: 6, and further including the amino acid substitutions corresponding to the following amino acid substitutions: (a) R55A; (b) E117A; (c) N46A/E117A; (d) Q116A/K124A/Q128A; (e) Q116A/K124D/Q128A; (f) E43A/Q116A/K124A/Q128A; (g) S45E/Q116A/K124A/Q128A; and (h) Q48A/Q116A/K124A/Q128A.

In some embodiments, the nucleic acids of the disclosure include an amino acid sequence having at least 70% sequence identity to SEQ ID NO: 6, and further including an amino acid substitution corresponding an amino acid residue selected from the group consisting of E43H, E43R, S45R, S45G, Q49S, Q49G, Q116W, Q116K, Q124Y, and Q128K.

In some embodiments, one or more amino acid substitution results in a tissue-selective IL-22 signaling compared to a reference IL-22 polypeptide lacking the one or more amino acid substitution. In some embodiments, the tissue-selective IL-22 signaling includes a reduction of IL-22 signaling in the skin while substantially retains IL-22 signaling in the pancreas and/or the gastrointestinal tract. In some embodiments, the tissue-selective IL-22 signaling includes a reduction of IL-22 signaling in the liver while substantially retains IL-22 signaling in the pancreas and/or the gastrointestinal tract.

In some embodiments, the one or more amino acid substitution results in a biased IL-22 signaling compared to a reference IL-22 polypeptide lacking the one or more amino acid substitution. In some embodiments, the biased IL-22 signaling includes a reduction in a STAT1-mediated pro-inflammatory function while substantially retains its STAT3-mediated function. In some embodiments, the biased IL-22 signaling includes a ratio of STAT1-mediated signaling to STAT3-mediated signaling ranging from 1:1.5 to 1:10. In some embodiments, the STAT3-mediated function is selected from the group consisting of tissue protection, tissue regeneration, cell proliferation, and cell survival. In some embodiments, the STAT1-mediated pro-inflammatory function is selected from the group consisting of cytokine production, chemokine production, and immune cell recruitment. In some embodiments, the STAT1-mediated pro-inflammatory function is reduced about 20% to about 100%. In some embodiments, the STAT1 signaling and/or STAT3 signaling is determined by an assay selected from the group consisting of by a gene expression assay, a phospho-flow signaling assay, and an enzyme-linked immunosorbent assay (ELISA).

In one aspect, some embodiments of the disclosure relate to recombinant nucleic acid molecules, wherein the nucleic acids include a nucleic acid sequence encoding a polypeptide that includes an amino acid sequence having at least 90% sequence identity to the amino acid sequence of the polypeptide of the disclosure.

Non-limiting exemplary embodiments of the disclosed nucleic acid molecules can include one or more of the following features. In some embodiments, the nucleic acid sequence is operably linked to a heterologous nucleic acid sequence. In some embodiments, the nucleic acid molecule is further defined as an expression cassette or an expression vector.

In one aspect, some embodiments of the disclosure relate to recombinant cells, wherein the recombinant cells include one or more of: (a) a recombinant polypeptide of the disclosure; and (b) a recombinant nucleic acid of the disclosure. In some embodiments, the recombinant cell is a eukaryotic cell. In some embodiments, the eukaryotic cell is a mammalian cell. In a related aspect, some embodiments of the disclosure relate to cell cultures including at least one recombinant cell of the disclosure and a culture medium.

In another aspect, some embodiments of the disclosure relate to methods for producing a polypeptide, wherein the methods include: (a) providing one or more recombinant cells of the disclosure; and (b) culturing the one or more recombinant cells in a culture medium such that the cells produce the polypeptide encoded by the recombinant nucleic acid molecule.

In some embodiments, the methods for producing a polypeptide of the disclosure further include isolating and/or purifying the produced polypeptide. In some embodiments, the methods for producing a polypeptide of the disclosure further include structurally modifying the produced polypeptide to increase half-life and/or extend duration of action of the polypeptide in vivo, e.g in a mammalian subject. In some embodiments, the modification includes one or more alterations selected from the group consisting of fusion to a human Fc antibody fragment, fusion to albumin, acylation, acetylation and PEGylation. Accordingly, in a related aspect, also provided herein are recombinant polypeptides produced by the method of the disclosure.

In one aspect, some embodiments of the disclosure relate to pharmaceutical compositions, wherein the pharmaceutical compositions include one or more of: (a) a recombinant polypeptide of the disclosure; (b) a recombinant nucleic acid of the disclosure; (c) a recombinant cell of the disclosure; and (d) a pharmaceutically acceptable carrier.

Non-limiting exemplary embodiments of the disclosed pharmaceutical compositions can include one or more of the following features. In some embodiments, the composition includes a recombinant polypeptide of the disclosure and a pharmaceutically acceptable carrier. In some embodiments, the composition includes a recombinant nucleic acid of the disclosure and a pharmaceutically acceptable carrier. In some embodiments, the composition comprises a recombinant viral vector comprising a nucleic acid sequence encoding the polypeptide of the disclosure. In some embodiments, the composition includes a recombinant cell comprising a nucleic acid encoding a polypeptide of the disclosure and a pharmaceutically acceptable carrier.

In one aspect, some embodiments of the disclosure relate to methods for modulating IL-22-mediated signaling in a subject, wherein the methods include administering to the subject a composition including one or more of: (a) a recombinant polypeptide of the disclosure; (b) a recombinant nucleic acid of the disclosure; (c) a recombinant cell of the disclosure; (d) a recombinant viral or non-viral vector comprising a nucleic acid of the disclosure, and (e) a pharmaceutically composition of the disclosure.

In another aspect, some embodiments of the disclosure relate to methods for the treatment of a disease, disorder or condition in a subject in need thereof, wherein the methods includes administering to the subject a composition including one or more of: (a) a recombinant polypeptide of the disclosure; (b) a recombinant nucleic acid of the disclosure; (c) a recombinant cell of the disclosure;(d) a recombinant viral or non-viral vector comprising a nucleic acid encoding a polypeptide of the disclosure; and (e) a pharmaceutically composition of the disclosure.

Non-limiting exemplary embodiments of the disclosed methods for modulating IL-22-mediated signaling in a subject and/or for the treatment of a condition in a subject in need thereof can include one or more of the following features. In some embodiments, the administered composition results in a tissue-selective IL-22 signaling in the subject compared to a reference IL-22 polypeptide lacking the one or more amino acid substitution. In some embodiments, the tissue-selective IL-22 signaling includes a reduction of IL-22 signaling in the skin while substantially retains IL-22 signaling in the pancreas and/or the gastrointestinal tract. In some embodiments, the tissue-selective IL-22 signaling includes a reduction of IL-22 signaling in the liver while substantially retains IL-22 signaling in the pancreas and/or the gastrointestinal tract.

In some embodiments, the administered composition results in a biased IL-22 signaling in the subject compared to a reference IL-22 polypeptide lacking the one or more amino acid substitution. In some embodiments, the biased IL-22 signaling includes a reduction in a STAT1-mediated pro-inflammatory function while substantially retains its STAT3-mediated function. In some embodiments, the biased IL-22 signaling includes a ratio of STAT1-mediated signaling to STAT3-mediated signaling ranging from 1:1.5 to 1:10. In some embodiments, the STAT3-mediated function is selected from the group consisting of tissue protection, tissue regeneration, cell proliferation, and cell survival. In some embodiments, the STAT1-mediated pro-inflammatory function is selected from the group consisting of cytokine production, chemokine production, and immune cell recruitment. In some embodiments, the STAT1-mediated pro-inflammatory function is reduced about 20% to about 100%, as determined by a gene expression assay, a phospho-flow signaling assay, and/or an enzyme-linked immunosorbent assay (ELISA).

In some embodiments, the administered composition results in a reduced capacity to induce expression of a pro-inflammatory gene selected from CXCL1, CXCL2, CXCL8, CXCL9, CXCL10, IL-1β, and IL-6 in the subject. In some embodiments, the administered composition substantially retains its capacity to induce expression of a gene selected from Reg3β, Reg3γ, Muc1, Muc2, Muc10, BCL-2, Cyclin-D, Claudin-2, LCN2, and β-Defensin in the subject. In some embodiments, the administration of the pharmaceutical composition does not inhibit T-cell activity in the subject.

In some embodiments, the administered composition enhances epithelial protection and regeneration. In some embodiments, the condition is an inflammatory disease, an immune disease or a chronic infection and diseases also. In some embodiments, the immune disease is an autoimmune disease. In some embodiments, the autoimmune disease is selected from the group consisting of rheumatoid arthritis, insulin-dependent diabetes mellitus, hemolytic anemias, rheumatic fever, thyroiditis, Crohn's disease, myasthenia gravis, glomerulonephritis, autoimmune hepatitis, multiple sclerosis, alopecia areata, psoriasis, vitiligo, dystrophic epidermolysis bullosa, systemic lupus erythematosus, graft vs. host disease, ulcerative colitis, pancreatitis, psoriatic arthritis, and diabetic foot ulcer. In some embodiments, the autoimmune disease is acute pancreatitis. In some embodiments, the subject is a mammal. In some embodiments, the mammal is a human. In some embodiments, the subject has or is suspected of having a condition associated with IL-22 mediated signaling.

In some embodiments, the composition is administered to the subject individually as a first therapy or in combination with a second therapy (e.g., an immunosuppressive agent, an immunosuppressant, or an anti-inflammatory agent). In some embodiments, the second therapy is selected from the group consisting of chemotherapy, radiotherapy, immunotherapy, hormonal therapy, toxin therapy, surgery, and disease modifying anti-rheumatic drugs (DMARDs). In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

In another aspect, some embodiments of the disclosure relate to kits for modulating IL-22-mediated signaling in a subject, or treating a condition in a subject in need thereof, wherein the kits include one or more of: a recombinant polypeptide of the disclosure; a recombinant nucleic acid of the disclosure; a recombinant cell of the disclosure; and a pharmaceutical composition of the disclosure.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIGS. 1A-1E schematically summarize the results of experiments performed to engineer an exemplary high-affinity IL-22 in accordance with some non-limiting embodiments of the disclosure via directed evolution approach. FIG. 1A depicts the structure of the human IL-22-IL22R1 partial complex (PDB ID: 3DLQ), with corresponding residues selected for randomization in mouse IL-22 site directed yeast-display library shown in red. FIG. 1B depicts a schematic strategy for yeast display-based affinity maturation of IL-22. MACS: magnetic activated cell sorting. FACS: fluorescence-activated cell sorting. FIG. 1C depicts histograms showing fluorescent intensity of Streptavidin-Alexa Fluor-647 (SA-647)-labeled IL10Rβ extracellular domain (ECD) binding to yeast-displayed wild-type IL-22 (WT), selected rounds of directed evolution, and the final high affinity IL-22 clone (super-22a). In these experiments, yeast were pre-bound with 1 μM unlabeled mIL22R1 (ECD). FIG. 1D illustrates that affinity matured IL-22 variants demonstrated enhanced binding to IL10Rβ. FIG. 1E illustrates that affinity matured IL-22 variants could elicit enhanced STAT3 signaling. Data are mean+/−SD for two independent replicates.

FIGS. 2A-2E depict the structure of the mouse IL-22/IL22R1/IL-10Rβ ternary complex. FIGS. 2A-2C depict three views of the 2.6 Å structure of the IL-22-Receptor complex showing IL-22 (super-22a) in yellow, IL22R1 in blue, and IL10Rβ in pink. FIG. 2D depicts a structural superposition of mouse IL-22-IL10Rβ complex with human IFN-λ-IL10Rβ (PDB ID:5T5W), showing ˜40 degree change in the relative orientation of IL10Rβ when bound to IL-22 compared to IFN-λ. FIG. 2E depicts the surface representations of IL-22 (yellow, left panel) and IFN-λ (green, right panel) bound to IL10Rβ, showing the different relative positions of the three aromatic residues in IL10Rβ that form key contacts in with both IL-22 and IFN-λ.

FIGS. 3A-3F illustrate a structure-guided design of biased IL-22 receptor agonists in accordance with some non-limiting embodiments of the disclosure. FIGS. 3B-3C depict two close-up views of the IL-22-IL10Rβ binding interface. Hydrogen bonds and salt-bridges are shown as black dashed-lines. Mutated residues in IL-22 resulting from affinity maturation are italicized. FIG. 3D illustrates that IL-22 variants that disrupted IL10Rβ-binding also uncoupled STAT3 and STAT1 signaling. Data are mean+/−SD for three independent replicates, shown as a percent of maximal WT IL-22 signal. FIG. 3E depicts normalized E max values for phospho-STAT3 and phospho-STAT1 calculated from sigmoidal dose response curves shown in FIG. 3DC. Data are mean+/−SD for three independent replicates. FIG. 3F illustrates that biased IL-22 variants could elicit activation of STAT3 but not STAT1 or STATS. Immunoblot for the indicated proteins in lysates prepared from HT-29 cells after stimulation with 100 nM wild-type IL-22 or indicated variants for 20 minutes.

FIGS. 4A-4I schematically summarize the results from experiments performed to demonstrate that an exemplary biased IL-22 variant (22-B3) in accordance with some non-limiting embodiments of the disclosure elicits tissue selective signaling activity. FIG. 4A depicts an experimental design for characterization of 22-B3 activity in vivo. In these experiments, mice (3 per group) were administered PBS or 200 μg recombinant WT IL-22 or 22-B3, and indicated organs were isolated after 30 minutes (protein analysis), 6 hours (RNA analysis) or 24 hours (serum analysis). FIGS. 4B-4E illustrate that 22-B3 elicits tissue specific phospho-STAT3 and phospho-STAT1 signaling activity. Tissue lysates from the indicated organs were analyzed by SDS-PAGE followed by immunoblot for the indicated proteins. Phospho-STAT1 signal in the skin was below the limit of detection for all samples. FIG. 4F illustrates that tissue specific 22-B3 signal strength correlates with IL10Rβ expression levels. The indicated organs were isolated from control mice, and relative expression levels of IL22R1 and IL10Rβ were analyzed by RT-qPCR (normalized to GAPDH). Data are mean+/−SEM for samples run in triplicate. FIGS. 4G-4H illustrate that biased IL-22 variants 22-B1, B2 and B3 elicit cell type selective signal activity in human cell lines in vitro. Panc-1 (FIG. 4G) or HepG2 (FIG. 4H) cells were treated with PBS or 100 nM WT IL-22 or the indicated variants for 20 minutes. Cell lysates were analyzed by SDS-PAGE followed by immunoblot for the indicated proteins. Phospho-STAT1 signal in Panc-1 cells was below the limit of detection for all samples. FIG. 4I illustrates that 22-B3 is a neutral antagonist in liver cells in vitro. HepG2 cells were incubated with 10 nM WT IL-22 and the indicated concentration of 22-B3 for 20 minutes. Cell lysates were analyzed by SDS-PAGE followed by immunoblot for the indicated proteins.

FIGS. 5A-5E schematically summarize the results from experiments performed to demonstrate that the biased IL-22 variant 22-B3 uncouples expression of tissue protective and pro-inflammatory IL-22 target genes in vivo. FIGS. 5A-5B illustrate that 22-B3 induces tissue protective gene expression in the pancreas and colon. RNA was isolated from the pancreas and colon of mice treated via intraperitoneal injection (I.P.) with PBS or 200 μg WT IL-22 or 22-B3 for 6 hours. Relative expression of target genes was analyzed by RT-qPCR (normalized to GAPDH). Data are mean+/−SEM for two independent biological replicates, each analyzed in triplicate (student's t-test, unpaired, *=p<0.05, **=p<0.01). FIGS. 5C-5E illustrate that 22-B3 does not induce expression of several pro-inflammatory genes in the colon, skin, and liver. RNA was isolated from the liver and skin (tail) of mice treated via I.P. injection with PBS or 200 μg WT IL-22 or 22-B3 6 hours. Relative expression of target genes was analyzed by RT-qPCR (normalized to GAPDH). Data are mean+/−SEM for two independent biological replicates, each analyzed in triplicate (student's t-test, unpaired, *=p<0.05, **=p<0.01).

FIGS. 6A-6D schematically summarize the results from experiments performed to demonstrate that biased IL-22 variant 22-B3 protects against acute pancreatitis without inducing systemic inflammation. FIG. 6A is a schematic depiction of acute pancreatitis regimen. FIG. 6B illustrates that 22-B3 retains protective effects in acute pancreatitis. Mice (5 per group) were pre-treated via I.P. injection with PBS or 50 μg WT IL-22 or 22-B3 at 20 hours and 2 hours before initiating pancreatitis via 6 hourly I.P. injections of Caerulein (50 μg/kg). Serum was isolated 1 hour after final Caerulein injection and levels of pancreatic amylase and lipase were analyzed (student's t-test, unpaired, *=p<0.05, **=p<0.01). FIG. 6C illustrates that 22-B3 does not induce hepatic acute phase response proteins in vivo. Mice (3 per group) were treated with PBS or the indicated amount of WT IL-22 or 22-B3 for 24 hours. Serum was isolated and levels of SAA-1/2 and Haptoglobin were analyzed by ELISA (student's t-test, unpaired, *=p<0.05, **=p<0.01). FIG. 6D schematically illustrates the differential signaling activity exhibited by 22-B3 in mouse pancreas (weak biased agonism), colon (strong biased agonism), skin (neutral antagonism) and liver (neutral antagonism).

FIG. 7A depicts a selection strategy for each round of IL-22 selections, indicating concentration of IL10Rβ ECD used and sorting method. MACS: magnetic activated cell sorting. FACS: fluorescence-activated cell sorting). FIG. 7B is a table showing the six residues targeted for randomization, the codons used for each site in library generation, and the amino acid present at each location in super-22a and super-22b. FIG. 7C depicts dose response curves for phospho-Y705-STAT3 (left panel) and phospho-Y701-STAT1 (right panel) in HT-29 (human, colorectal) cells stimulated with WT IL-22 or indicated variants for 20 minutes and analyzed by flow cytometry following fixation and permeabilization with paraformaldehyde/methanol. Data are mean+/−SD for two independent replicates.

FIGS. 8A-8B depict size exclusion chromatography traces for purified Super-22a (FIG. 8A) and Super-22b (FIG. 8B) bound IL-22 Receptor complexes and accompanying Coomassie-stained SDS-PAGE gels. Fractions used for crystallization trials are indicated in red. FIG. 8C is a table showing the mutations present in the IL-22, IL10Rβ, and IL22R1 constructs used for crystallization. FIG. 8D depicts a close-up view of the stem contacts between IL22R1 and IL10Rβ present in the ternary complex.

FIG. 9A illustrates dose response curves for phospho-Y705-STAT3 (top) and phospho-Y701-STAT1 (bottom) in HT-29 (human, colorectal) cells stimulated with WT IL-22 or indicated variants for 20 minutes and analyzed by flow cytometry following fixation and permeabilization with paraformaldehyde/methanol. Data are mean+/−SD for two independent replicates. FIG. 9B is a table showing the mutations present in several biased mouse IL-22 variants (22-B1-B5). FIG. 9C depicts normalized Eₘₐₓ values for phospho-STAT3 and phospho-STAT1 calculated from sigmoidal dose-response curves from HT-29 cells stimulated with WT human IL-22 or indicated human IL-22 variants for 20 minutes and analyzed by flow cytometry. Data are mean+/−SD for two replicates. FIG. 9D is a table showing the mutations present in several biased human IL-22 variants (h22-B1-B3)

FIG. 10 depicts a sequence alignment of human IL-22 polypeptide (SEQ ID NO: 1) and murine IL-22 polypeptide (SEQ ID NO: 6). In the alignment, identical amino acids or conserved amino acid substitutions among aligned sequences are identified by asterisks. The alignment figure provided herein was generated using the program CLUSTAL version 1.2.4.

FIG. 11A: HT-29 (human colorectal carcinoma) cells were treated with varying concentrations of wild-type or mutant mouse IL-22 for 20 minutes. Cells were fixed and permeabilized using Methonol/PFA and stained with fluorescently conjugated anti-phospho-STAT1 (AF488) or anti-phospho-STAT3 (AF647) antibodies, and fluorescent intensities were analyzed by flow cytometry. Dose-response curves. Data were fit to a sigmoidal dose-response curve allowing calculation of the E max for pSTAT1 and pSTAT3 signaling. E max for biased variants were normalized to percentages of wild type IL22. Data shown are mean+/−SEM. FIG. 11B: Graph showing the ratio of the phospho-STAT3/phospho-STAT1 E max for each IL-22 variant, using the data from (A). FIG. 12C: List of mouse IL-22 variants and corresponding mutations relative to wild-type mouse IL-22. FIG. 11C is a table showing the mutations present in biased mouse IL-22 variants shown in A and B.

FIGS. 12A-12B: HT-29 (human colorectal carcinoma; FIG. 13A) or HepG2 (human liver; FIG. 13B) cells were treated with varying concentrations of wild-type or mutant mouse IL-22 for 20 minutes. Cells were fixed and permeabilized using Methonol/PFA and stained with fluorescently conjugated anti-phospho-STAT1 (AF488) or anti-phospho-STAT3 (AF647) antibodies, and fluorescent intensities were analyzed by flow cytometry. Dose-response curves. Data were fit to a sigmoidal dose-response curve allowing calculation of the E max for pSTAT1 and pSTAT3 signaling. E max for biased variants were normalized to percentages of wild type IL22. Data shown are mean+/−SEM for 3 independent replicates. FIG. 13C: List of human IL-22 variants and corresponding mutations relative to wild-type human IL-22.

FIG. 13A shows effect of biased IL-22 variant 22-B3 on the phosphorylation of JAK1 and TYK2 in HT-29 cells. Immunoblot for the indicated proteins in lysates prepared from HT-29 cells stimulated with 100 nM WT IL-22 or the indicated variants for 20 minutes. FIGS. 13B and C depict differences in IL-22 receptor tyrosine phosphorylation induced by biased IL-22 variants 22-B1, B2, and B3. Immunoblots for the indicated proteins in lysates and anti-HA immunoprecipitates prepared from HEK-293T cells transiently expressing the indicated HA-IL22Rα constructs and stimulated with 10 nM WT IL-22 or the indicated variants for 20 minutes. FIG. 13D is a model of how IL-22 variants induce biased STAT signaling by exploiting the distinct mechanisms of STAT3 and STAT1 activation downstream of IL-22Rα.

### DETAILED DESCRIPTION OF THE DISCLOSURE

The present disclosure generally relates to, inter alia, compositions and methods for selectively modulating signal transduction pathway mediated by interleukin 22 (IL-22) in a subject. As described in greater detail below, IL-22-mediated signaling can be modulated via biased agonism of STAT1-mediated pro-inflammatory function and/or STAT3-mediated signaling. More particularly, in some embodiments, the disclosure provides IL-22 polypeptide variants with modulated binding affinity for the natural ligands of IL-22, e.g., interleukin 10 receptor subunit beta (IL10Rβ). Some embodiments of the disclosure provide IL-22 partial agonists that possess tissue-selective IL-22 signaling. Some embodiments of the disclosure provide IL-22 partial agonists that possess biased IL-22 signaling, for example confer a reduction in a STAT1-mediated pro-inflammatory function while substantially retains its STAT3-mediated function. The disclosure also provides compositions and methods useful for producing such IL-22 polypeptide variants, methods for modulating IL-22-mediated signaling in a subject, as well as methods for the treatment of conditions associated with perturbations of signal transduction downstream of the IL-22 receptor.

Interleukin-22 (IL-22) is a member of the IL-10 family of cytokine that is produced by Th22 cells, NK cells, lymphoid tissue inducer (LTi) cells, dendritic cells and Th17 cells. IL-22 binds to the IL-22R1/IL-10Rβ receptor complex, which is expressed in innate cells such as epithelial cells, hepatocytes, and keratinocytes and in barrier epithelial tissues of several organs including dermis, pancreas, intestine and the respiratory system. IL-22 acts on epithelial cells to promote tissue protection and regeneration in response to inflammation. Although the ability of IL-22 to counteract inflammatory damage without suppressing immune function has made it an attractive therapeutic target, IL-22 can be pro-inflammatory in some contexts.

The present disclosure provides, inter alia, novel IL-22 compositions which are based on new insights into how IL-22 interacts with its cognate receptors, in particular, IL-10Rβ. In particular, the experimental data presented below surprisingly revealed that mutations in the IL-22 binding site for IL-10Rβ result in biased agonists capable of selectively STAT1-mediated pro-inflammatory function and/or STAT3-mediated signaling in a tissue-dependent manner. Further, these IL-22 biased agonists can confer a reduction in STAT1-mediated pro-inflammatory functions while substantially retains its STAT3-mediated functions. In some cases, the experimental data described herein indicate that the biased IL-22 variants of the disclosure will be at least as effective as wild-type (WT) recombinant IL-22 in all of these applications, with significant reduction in side effects such as inflammation of the skin and liver. As such, these molecules can be used to treat autoimmune disorders and conditions.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof “A and/or B” is used herein to include all of the following alternatives: “A”, “B”, “A or B”, and “A and B”.

The term “about”, as used herein, has its ordinary meaning of approximately. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

The terms “administration” and “administering”, as used herein, refer to the delivery of a bioactive composition or formulation by an administration route comprising, but not limited to, oral, intravenous, intra-arterial, intramuscular, intraperitoneal, subcutaneous, intramuscular, and topical administration, or combinations thereof. The term includes, but is not limited to, administering by a medical professional and self-administering.

The terms “cell”, “cell culture”, and “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the original cell, cell culture, or cell line.

The term “effective amount”, “therapeutically effective amount”, or “pharmaceutically effective amount” of a subject recombinant polypeptide of the disclosure generally refers to an amount sufficient for a composition to accomplish a stated purpose relative to the absence of the composition (e.g., achieve the effect for which it is administered, treat a disease, reduce a signaling pathway, or reduce one or more symptoms of a disease or health condition). An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “IL-22” means wild-type IL-22, whether native or recombinant. As such, an IL-22 polypeptide refers to any IL-22 polypeptide, including but not limited to, a recombinant produced IL-22 polypeptide, synthetically produced IL-22 polypeptide, IL-22 extracted from cells or tissues. An amino acid sequence of wild-type human IL-22 precursor is depicted in SEQ ID NO: 1, which is a 179 amino acid residue protein with an N-terminal 33 amino acid signal peptide that can be removed to generate a 146 amino acid mature protein. The amino acid sequence of the mature human IL-22 is provided in SEQ ID NO: 34. An amino acid sequence of wild-type murine (Mus musculus) IL-22 precursor is depicted in SEQ ID NO: 6, which is a 179 amino acid residue protein with an N-terminal 33 amino acid signal peptide that can be removed to generate 146 amino acid mature protein that shares approximately 79% sequence identity with human IL-22 protein. For the purpose of the present disclosure, all amino acid numbering is based on the precursor polypeptide (or pre-protein) sequence of the IL-22 protein set forth in SEQ ID NO: 1 (human IL-22) or SEQ ID NO: 6 (mouse IL-22). However, one of skill in the art would understand that mature proteins are often used to generate recombinant polypeptide constructs. The amino acid sequence of the mature murine IL-22 is provided in SEQ ID NO: 35.

As used herein, the term “variant” of an IL-22 polypeptide refers to a polypeptide in which one or more amino acid substitutions, deletions, and/ or insertions are present as compared to the amino acid sequence of a reference IL-22 polypeptide, e.g., a wild-type IL-22 polypeptide. As such, the term “IL-22 polypeptide variant” includes naturally occurring allelic variants or alternative splice variants of an IL-22 polypeptide. For example, a polypeptide variant includes the substitution of one or more amino acids in the amino acid sequence of a parent IL-22 polypeptide with a similar or homologous amino acid(s) or a dissimilar amino acid(s).

The term “operably linked”, as used herein, denotes a physical or functional linkage between two or more elements, e.g., polypeptide sequences or polynucleotide sequences, which permits them to operate in their intended fashion. For example, an operably linkage between a polynucleotide of interest and a regulatory sequence (for example, a promoter) is functional link that allows for expression of the polynucleotide of interest. It should be understood that, operably linked elements may be contiguous or non-contiguous. In the context of a polypeptide, “operably linked” refers to a physical linkage (e.g., directly or indirectly linked) between amino acid sequences (e.g., different domains) to provide for a described activity of the polypeptide. In the present disclosure, various domains of the recombinant polypeptides of the disclosure may be operably linked to retain proper folding, processing, targeting, expression, binding, and other functional properties of the recombinant polypeptides in the cell. Operably linked domains of the recombinant polypeptides of the disclosure may be contiguous or non-contiguous (e.g., linked to one another through a linker).

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.gov/BLAST. Such sequences are then said to be “substantially identical.” This definition also refers to, or may be applied to, the complement of a sequence. This definition also includes sequences that have deletions and/or additions, as well as those that have substitutions. Sequence identity can be calculated using published techniques and widely available computer programs, such as the GCS program package (Devereux et al, Nucleic Acids Res. 12:387, 1984), BLASTP, BLASTN, FASTA (Atschul et al., J Mol Biol 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

The term “pharmaceutically acceptable excipient” as used herein refers to any suitable substance that provides a pharmaceutically acceptable carrier, additive or diluent for administration of a compound(s) of interest to a subject. As such, “pharmaceutically acceptable excipient” can encompass substances referred to as pharmaceutically acceptable diluents, pharmaceutically acceptable additives, and pharmaceutically acceptable carriers. As used herein, the term “pharmaceutically acceptable carrier” includes, but is not limited to, saline, solvents, dispersion media, coatings, antibacterial and antifungal agents, isotonic and absorption delaying agents, and the like, compatible with pharmaceutical administration. Supplementary active compounds (e.g., antibiotics and additional therapeutic agents) can also be incorporated into the compositions.

The term “recombinant” or “engineered” nucleic acid molecule as used herein, refers to a nucleic acid molecule that has been altered through human intervention. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. As non-limiting examples, a recombinant nucleic acid molecule can be one which: 1) has been synthesized or modified in vitro, for example, using chemical or enzymatic techniques (for example, by use of chemical nucleic acid synthesis, or by use of enzymes for the replication, polymerization, exonucleolytic digestion, endonucleolytic digestion, ligation, reverse transcription, transcription, base modification (including, e.g., methylation), or recombination (including homologous and site-specific recombination)) of nucleic acid molecules; 2) includes conjoined nucleotide sequences that are not conjoined in nature; 3) has been engineered using molecular cloning techniques such that it lacks one or more nucleotides with respect to the naturally occurring nucleic acid molecule sequence; and/or 4) has been manipulated using molecular cloning techniques such that it has one or more sequence changes or rearrangements with respect to the naturally occurring nucleic acid sequence. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. Another non-limiting example of a recombinant nucleic acid and recombinant protein is an IL-22 polypeptide variant as disclosed herein.

As used herein, an “individual” or a “subject” includes animals, such as human (e.g., human individuals) and non-human animals. In some embodiments, an “individual” or “subject” is a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

The term “vector” is used herein to refer to a nucleic acid molecule or sequence capable of transferring or transporting another nucleic acid molecule. The transferred nucleic acid molecule is generally linked to, e.g., inserted into, the vector nucleic acid molecule. Generally, a vector is capable of replication when associated with the proper control elements. The term “vector” includes cloning vectors and expression vectors, as well as viral vectors and integrating vectors. An “expression vector” is a vector that includes a regulatory region, thereby capable of expressing DNA sequences and fragments in vitro and/or in vivo. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses. In some embodiments, a vector is a gene delivery vector. In some embodiments, a vector is used as a gene delivery vehicle to transfer a gene into a cell.

It is understood that aspects and embodiments of the disclosure described herein include “comprising”, “consisting”, and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

Headings, e.g., (a), (b), (i) etc., are presented merely for ease of reading the specification and claims. The use of headings in the specification or claims does not require the steps or elements be performed in alphabetical or numerical order or the order in which they are presented.

As will be understood by one having ordinary skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to”, “at least”, “greater than”, “less than”, and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub- combination was individually and explicitly disclosed herein.

### Interleukin-22 (IL-22) and Inflammatory Immune Responses

Interleukin-22 (IL-22) is a member of the IL-10 family of cytokine that is produced by Th22 cells, NK cells, lymphoid tissue inducer (LTi) cells, dendritic cells and Th17 cells. IL-22 binds to the IL-22R1/IL-10Rβ receptor complex, which is expressed in non-hematopoietic cells such as epithelial cells, hepatocytes, and keratinocytes and in barrier epithelial tissues of several organs including dermis, pancreas, intestine and the respiratory system.

It has been reported that IL-22 plays an important role in mucosal immunity, mediating early host defense against attaching and effacing bacterial pathogens. IL-22 promotes the production of anti-microbial peptides and pro-inflammatory cytokines from epithelial cells and stimulates proliferation and migration of colonic epithelial cells in the gut. It was reported previously that upon bacterial infection, IL-22 knock-out mice displayed impaired gut epithelial regeneration, high bacterial load and increased mortality. Similarly, infection of IL-22 knock-out mice with influenza virus resulted in severe weight loss and impaired regeneration of tracheal and bronchial epithelial cells. Thus, IL-22 plays a pro-inflammatory role in suppressing microbial infection as well as an anti-inflammatory protective role in epithelial regeneration in inflammatory responses.

Inflammatory immune responses are essential for protecting organisms against infection and disease. However, excessive immune cell activation often results in damage to bystander tissues, contributing to organ malfunction, chronic inflammation, and autoimmune disease. In order to prevent and mitigate these effects, the immune system has evolved a number of regulatory mechanisms that restrain inflammation and prevent aberrant tissue damage, especially through the production of immunoregulatory cytokines and other secreted factors.

One important secreted factor is the cytokine IL-22, which plays a central role in maintaining tissue homeostasis. During periods of inflammation, multiple immune cell types including CD4⁺ T cells and Type 3 innate lymphoid cells (ILC3s) produce IL-22 at barrier interfaces, where it acts on epithelial cells to enhance tissue integrity and promote regeneration. IL-22 exerts these protective effects on a variety of tissues including the pancreas, GI tract, liver, and thymus, and treatment with exogenous IL-22 is protective in mouse models of pancreatitis, inflammatory bowel disease (IBD), and acute liver damage. Moreover, IL-22 protects against graft-versus host disease (GvHD)-mediated intestinal damage by promoting the survival and regenerative capacity of Lgr5⁺ intestinal stem cells. The unique ability of IL-22 to prevent and reverse inflammatory tissue damage without suppressing immune function makes it an appealing therapeutic target for autoimmune diseases and distinguishes it from current treatment modalities, such as corticosteroids or cytokine antagonists.

Despite these beneficial functions, IL-22 can also be pathogenic in some contexts as well, most notably in IBD and psoriasis. The pro-inflammatory effects of IL-22 are due in large part to the induction neutrophil recruiting chemokines (e.g. CXCL1) in the skin, liver and GI tract, as well as production of acute-phase response proteins such as Serum-Amyloid A (SAA)-1/2, which promotes activation of inflammatory Th₁₇ cells. Indeed, administration of exogenous IL-22 induces a significant increase in the serum levels acute-phase response proteins in both mice and humans. This potential of IL-22 to drive both local and systemic inflammation therefore presents a significant limitation to its clinical utility.

Mechanistically, IL-22 signals through a heterodimeric receptor consisting of a high affinity subunit, IL22R1, and a low affinity subunit called IL10Rβ. Whereas IL10Rβ is expressed ubiquitously, IL-22R1 is expressed primarily on epithelial cells, dictating the target cell specificity of IL-22. Upon binding to IL22R1, IL-22 facilitates the dimerization of IL22R1 and IL10Rβ, bringing together the intracellular, receptor associated kinases Jak1 and Tyk2, which phosphorylate both each other as well as tyrosines on intracellular domain (ICD) of IL22R1. These phospho-tyrosines in turn recruit the STAT1 and STAT3 transcription factors, facilitating their phosphorylation and activation. While expression of the tissue protective and regenerative genes downstream of IL-22 are mediated by STAT3, several of the pro-inflammatory effects of IL-22 are thought to be due to STAT1, which controls the expression of pro-inflammatory interferon stimulated genes (ISGs).

Functionally selective, or “biased”, agonists that uncouple downstream signaling responses have been extensively characterized for G-protein coupled receptors (GPCRs), and recent studies suggest that such agonists are possible for cytokine receptors as well. However, these approaches rely on extensive structural information, which is currently lacking for the IL-22 receptor complex. This is due primarily to the extremely low affinity of IL-22 for its shared receptor subunit, IL10Rβ, which hinders complex assembly in vitro.

As described in the Examples below, a directed evolution approach has been used to engineer an IL-22 variant with enhanced affinity for IL10Rβ, enabling us to solve the crystal structure of the IL-22/IL22R1/IL10Rβ ternary complex to 2.6 Å resolution. Structure-guided mutations in IL-22 targeting the IL-10Rβ interface resulted in biased IL-22 variants that signal through STAT3 but not STAT1. An exemplary IL-22 polypeptide variant disclosed herein, 22-B3, also elicited tissue-selective signaling responses in vivo, acting as a STAT3-biased agonist in the pancreas and colon but a neutral antagonist in the skin and liver. In particular, 22-B3 retains the tissue protective functions of IL-22 in vivo without inducing inflammatory mediators in the skin, liver, and colon, thereby uncoupling the major tissue protective and pro-inflammatory functions of IL-22.

Cytokines play numerous important roles in controlling host immune responses and promoting tissue homeostasis, but often also exert pleiotropic or counterproductive effects that can limit their use as therapeutics. As a result, while cytokine and cytokine receptor antagonists have achieved significant clinical success, there are far fewer such examples for cytokine receptor agonists. The cytokine IL-22 is a prime example of a cytokine that can exert both beneficial and deleterious functions depending on the tissue and disease context. As described in the greater detail below, a structure-guided approach has been used to develop functionally selective variants of IL-22 that effectively uncouple the tissue protective and pro-inflammatory functions of IL-22, revealing new insights into how IL-22 exerts these distinct functions, while also providing a path for the development of improved cytokine-based therapeutics.

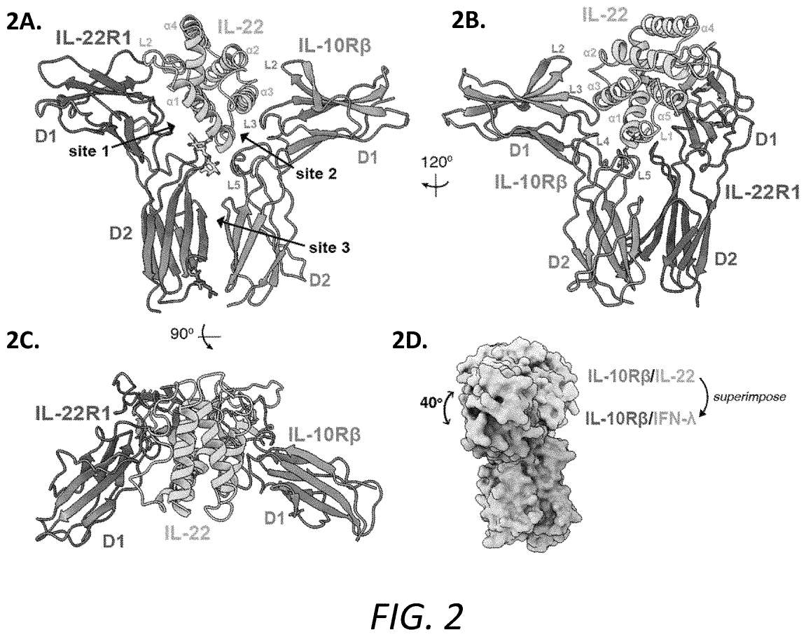

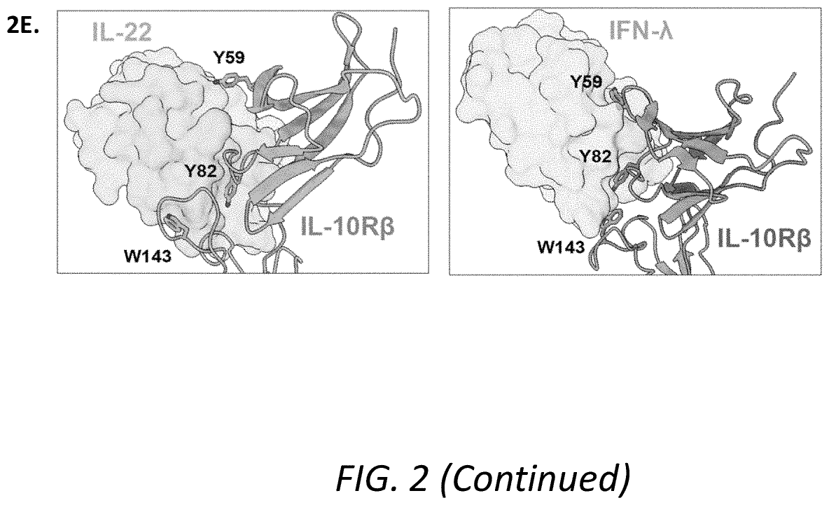

A primary barrier preventing previous structural characterization of the IL-22 receptor complex was the presence of extremely low affinity interactions between subunits, specifically IL-22 and IL10Rβ. A ligand engineering approach was used here to obtain information relating to the ternary receptor complex. The structure of the IL-22 Receptor complex presented here provides several insights into the mechanism of receptor sharing within the IL-10 superfamily. The IL-22 receptor complex is the second reported structure of a cytokine bound to the shared receptor subunit IL10Rβ, and reveals a distinct mode of ligand engagement compared to the previously solved structure of the IFN-λ complex (FIG. 2. In addition to IL-22 and IFN-λ, IL10Rβ is also present in the IL-10 and IL-26 Receptors, and the structural characterization of these complexes in relation to the mechanisms underlying receptor sharing by these cytokines.

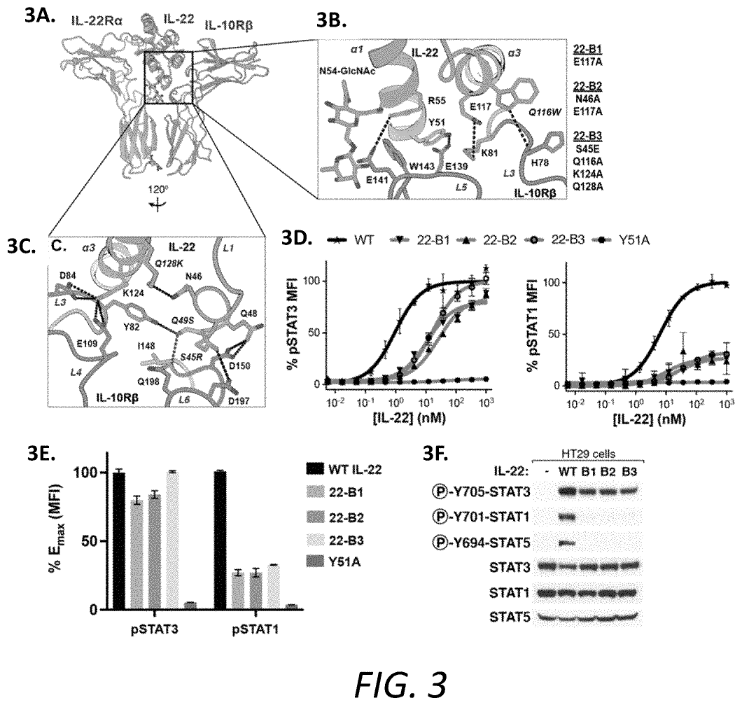

Analyzing the IL-22/IL10Rβ binding site enabled to the generation of a series of human and murine IL-22 variants with amino acid substitutions at the IL10Rβ binding interface with IL-22 that resulted in IL22 variants possessing STAT3-biased agonism. Although previous examples of engineered cytokine receptor ligands with biased activity have relied on altering receptor topology, the experimental results described below demonstrate that moculation of the affinity of a natural cytokine for its receptor is sufficient to generate biased agonism. Mechanistically, the IL22variants of the present disclosure induce substantially reduced phosphorylation of IL22R1 relative to WT IL-22, thereby exploiting the two distinct modes of activation of STAT1 and STAT3. Whereas STAT1 is recruited to the IL22 receptor by binding to phospho-tyrosines on the ICD of IL22R1, STAT3 can pre-associate with IL22R1 independent of phospho-tyrosine binding, and therefore be activated even in the complete absence of receptor phosphorylation (FIG. 3F).

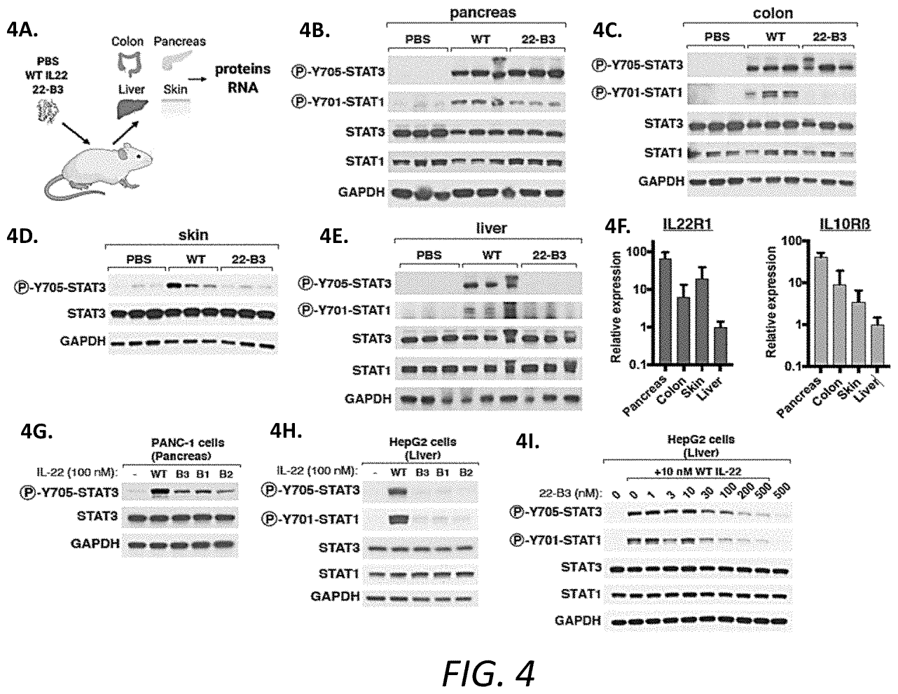

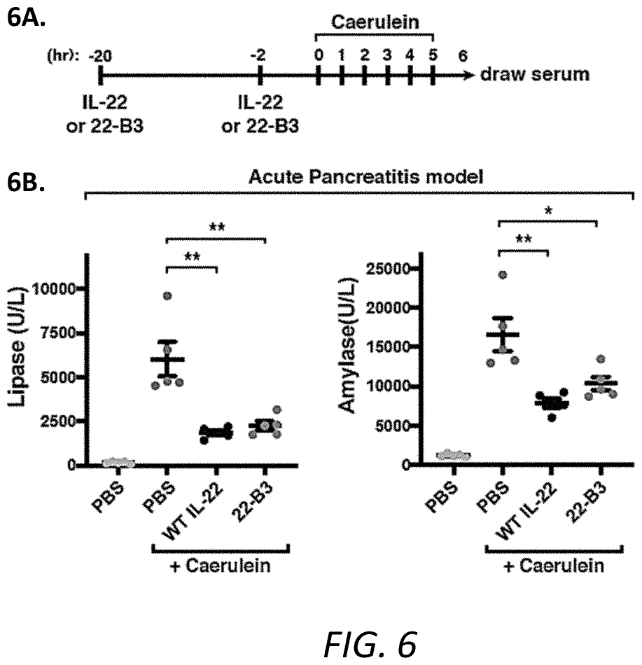

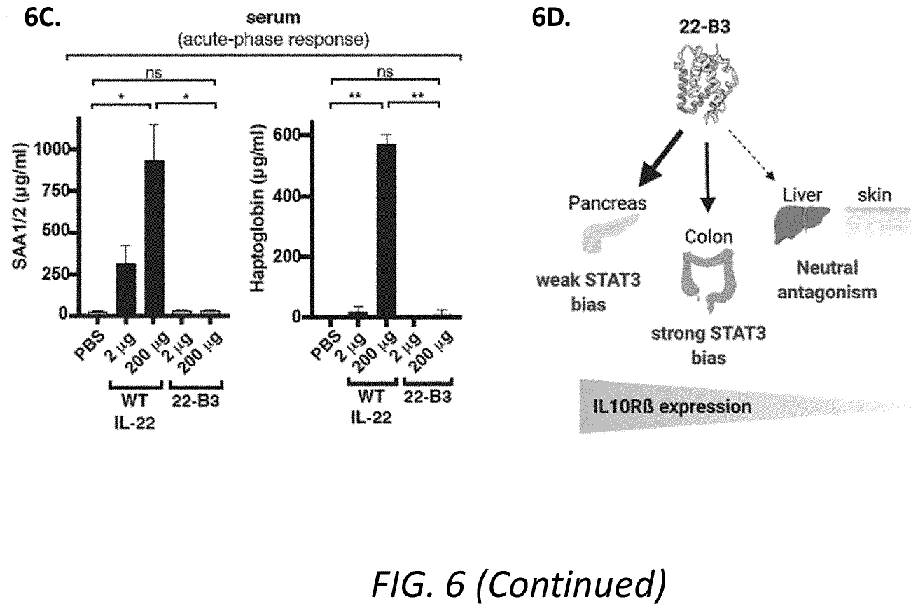

Characterization of the biased IL-22 variant 22-B3 in vivo revealed tissue selective signaling activities as well, with relative signal strength correlating with IL10Rβ expression levels (FIG. 6D). It is worth noting that this is not a result of altered tissue “targeting”, as IL-22 is still able to bind to IL-22R1 on the surface of liver cells in vitro (FIG. 4I), but rather is an example of functional tissue selectivity. The concept of tissue selectivity is well established in GPCR and nuclear receptor pharmacology, with the most notable example being Estrogen Receptor agonists (Riggs and Hartmann, 2003), and the data presented here extends this concept to cytokine receptor signaling as well.

Without being bound to any particular theory, the experimental data described herein demonstrates that a combination of affinity for IL10Rβ and level of IL10Rβ expression determines the type of signaling induced by IL-22 on a given cell. This signal strength exists on a spectrum ranging from full agonism to neutral antagonism, with intermediate signal strength resulting in biased activity leading to the phosphorylation of STAT3 but not STAT1. The ability of 22-B3 to uncouple the tissue protective and pro-inflammatory functions of IL-22 in vivo appears to result from a combination of both its biased signaling activity and tissue selectivity. For example, by activating STAT3 but not STAT1 in the colon, 22-B3 retains the ability to induce expression of STAT3-dependent tissue protective genes such as Reg3β/γ and Muc1, but no longer induces the pro-inflammatory STAT1-target gene CXCL1. However, acute phase response proteins such as SAA-1/2 are STAT3-target genes, and therefore the lack of a systemic increase in these proteins is instead due to the complete inactivity of 22-B3 in the liver. It is worth noting that due to its inability to activate STAT3 in the skin and liver, 22-B3 does not retain the tissue protective effects of WT IL-22 in these tissues. The experimental data described herein demonstrate that 22-B3 or similar IL-22 variants have therapeutic utility in the treatment of inflammatory diseases in the pancreas and GI tract, with significantly reduced risk of side effects associated with inflammation in the skin and liver.

As described in greater detail below, in order to gain mechanistic insight into these opposing effects of IL-22 signaling, several experiments were performed using yeast display-based directed evolution to engineer high affinity IL-22 variants, which in turn have enabled the structural characterization of IL-22 bound to its heterodimeric receptor complex. By revealing how IL-22 engages its low-affinity receptor subunit, IL10Rβ, the crystal structure of the heteromeric IL-22/IL22R1/IL10Rβ complex described herein was employed in the design of IL-22 polypeptide variants with one or more amino acid substitutions at the IL10Rβ-binding interface that are capable of modulating the signal transduction pathway downstream of the IL-22 receptor. It was found that several of these IL-22 variants displayed strong biased agonism in colonic epithelial cells by exploiting the two distinct mechanisms of STAT1 and STAT3 activation. In particular, an exemplary variant of these variants, 22-B3, also elicited tissue-selective signaling responses in vivo, driving expression of tissue protective genes in the pancreas and GI tract without inducing systemic inflammation.

### Compositions of the Disclosure

### A. Recombinant IL-22 Polypeptides

As outlined above, some embodiments of the disclosure relate to IL-22 polypeptide variants engineered to modulate STAT signaling downstream of the IL-22 receptor, e.g., capable of conferring STAT1-mediated pro-inflammatory functions while substantially retains its STAT3-mediated function in a tissue-specific manner. For example, in some embodiments of the disclosure, the IL-22 polypeptide variants confer a reduction of IL-22 signaling in the skin while substantially retains IL-22 signaling in the pancreas and/or the gastrointestinal tract. In some other embodiments, the IL-22 polypeptide variants of the disclosure confer a reduction of IL-22 signaling in the liver while substantially retains IL-22 signaling in the pancreas and/or the gastrointestinal tract.

In one aspect, some embodiments of the disclosure relate to recombinant polypeptides that include: (a) an amino acid sequence having at least 70% sequence identity to an interleukin 22 (IL-22) polypeptide having the amino acid sequence of SEQ ID NO: 1, and further including (b) one or more amino acid substitutions in the sequence of SEQ ID NO: 1.

Non-limiting exemplary embodiments of the recombinant polypeptides disclosed herein can include one or more of the following features. In some embodiments, the recombinant polypeptides include an amino acid sequence having at least 75%, at least 80%, at least 85%, at least 90%, at least 95%, at least 96%, at least 97%, at least 98%, or at least 99% to the sequence of SEQ ID NO: 1. In some embodiments, the recombinant polypeptides include an amino acid sequence having 100% sequence identify to the sequence of SEQ ID NO: 1.

In some embodiments, the amino acid sequence of the recombinant polypeptides disclosed herein further include one or more amino acid substitutions at a position corresponding to an amino acid residue selected from the group consisting of X43, X49, X45, X46, X116, X124, and X128 of SEQ ID NO: 1. In some embodiments, the amino acid sequence of the recombinant polypeptides further include 1 to 3, 2 to 5, 3 to 6, or 4 to 7 amino acid substitutions at a position corresponding to an amino acid residue selected from the group consisting of X43, X49, X45, X46, X116, X124, and X128 of SEQ ID NO: 1. Exemplary IL-22 polypeptide variants according to this aspect can include substitutions of 1, 2, 3, 4, 5, 6, 7, 8, 9, 10 or more amino acids in the sequence of SEQ ID NO: 1. In some embodiments, the amino acid sequence of the recombinant polypeptides further include at least 1, at least 2, at least 3, at least 4, at least 5, at least 6, or at least 7 amino acid substitutions at a position corresponding to an amino acid residue selected from the group consisting of X43, X49, X45, X46, X116, X124, and X128 of SEQ ID NO: 1.

In some embodiments, the amino acid sequence of the recombinant polypeptides further include one or more additional amino acid substitutions at a position corresponding to an amino acid residue selected from the group consisting of X48, X55, and X117 of SEQ ID NO: 1. In some embodiments, the amino acid sequence of the recombinant polypeptides further include one additional amino acid substitution at a position corresponding to an amino acid residue selected from the group consisting of X48 of SEQ ID NO: 1. In some embodiments, the amino acid sequence of the recombinant polypeptides further include one additional amino acid substitution at a position corresponding to an amino acid residue selected from the group consisting of X55 of SEQ ID NO: 1. In some embodiments, the amino acid sequence of the recombinant polypeptides further include one additional amino acid substitution at a position corresponding to an amino acid residue selected from the group consisting of X117 of SEQ ID NO: 1.

In some embodiments, the amino acid substitution(s) is at a position corresponding to an amino acid residue selected from the group consisting of X43, X45, X46, X48, X49, X55, and X117 of SEQ ID NO: 1. In some embodiments, the polypeptides of the disclosure further include a combination of amino acid substitutions at positions corresponding to amino acid residues X116, X124, X128 of SEQ ID NO: 1. In some embodiments, the amino acid sequence includes an amino acid substitution corresponding to amino acid residue X55 or X117 of SEQ ID NO: 1.

In accordance with this disclosure, any such substitutions in an IL-22 polypeptide result in an IL-22 variant that has an altered binding affinity for IL10Rβ and/or IL-22R1, relative to the binding affinity of the parent IL-22 polypeptide lacking such substitutions. For example, the IL-22 polypeptide variants disclosed herein can have increased affinity or decreased affinity for IL-22R1 and/or IL10Rβ or can have an affinity for these receptors identical or similar to that of wild-type IL-22. The IL-22 polypeptide variants disclosed herein can also include conservative modifications and substitutions at other positions of IL-22 (e.g., those that have a minimal effect on the secondary or tertiary structure of the IL-22 variants). Such conservative substitutions include those described by Dayhoff in The Atlas of Protein Sequence and Structure 5 (1978), and by Argos in EMBO J, 8:779-785 (1989). For example, amino acids belonging to one of the following groups represent conservative changes: Group I: Ala, Pro, Gly, Gln, Asn, Ser, Thr; Group II: Cys, Ser, Tyr, Thr; Group III: Val, Ile, Leu, Met, Ala, Phe; Group IV: Lys, Arg, His; Group V: Phe, Tyr, Trp, His; and Group VI: Asp, Glu.

In some embodiments, the amino acid substitution(s) in the amino acid sequence of the recombinant IL-22 polypeptides disclosed herein is independently selected from the group consisting of an alanine substitution, an arginine substitution, an aspartic acid substitution, a histidine substitution, a glutamic acid substitution, a lysine substitution, a serine substitution, a tryptophan substitution, and combinations of any thereof. Non-limiting examples of the amino acid substitutions in the recombinant IL-22 polypeptides disclosed herein are provided in Tables 1 and 2 below.

TABLE 1
Exemplary amino acid substitutions in the recombinant
IL-22 polypeptides of the disclosure.

| Position of | Original | Exemplary substitute amino |
|---|---|---|
| SEQ ID NO: 1 | amino acid | acid |
| 43 | D | A, R, N, Q, I, L, M, S, T, V, K, H |
| 45 | S | E, A, R, N, D, Q, E, I, L, K, M, V, G |
| 46 | N | A, R, D, E, I, K, K, M, S, V |
| 48 | Q | A, R, D, E, I, L, K, M, S, T, V |
| 49 | Q | A, R, D, E, I, L, K, M, S, T, V, G |
| 55 | R | A, N, D, Q, E, I, L, M, S, T, V |
| 116 | Q | A, R, D, E, I, L, K, M, S, T, V, W |
| 117 | E | A, R, N, Q, I, L, V, K, M, S, T |
| 124 | R | A, N, D, Q, E, I, L, M, S, T, V, Y |
| 128 | R | A, N, D, Q, E, I, L, M, S, T, V, K |

In some embodiments, the amino acid substitution(s) is at a position corresponding to an amino acid residue selected from the group consisting of D43, S45, N46, Q49, Q116, R124, and R128 of SEQ ID NO: 1. In some embodiments, the polypeptides of the disclosure include an amino acid sequence having at least 70% sequence identity to SEQ ID NO: 1, and further include the amino acid substitutions corresponding to the following amino acid substitutions: (a) R55A; (b) E117A; (c) N46A/E117A; (d) Q116A/R124A/R128A; (e) Q116A/R124D/R128A; (f) D43A/Q116A/R124A/R128A; (g) S45E/Q116A/R124A/R128A; and (h) Q48A/Q116A/R124A/R128A. In some embodiments, the polypeptides of the disclosure include an amino acid sequence having at least 75%, at least 80%, at least 85%, at least 90%, at least 95%, at least 96%, at least 97%, or at least 98%, at least 99% sequence identity to SEQ ID NO: 1, and further include the amino acid substitutions corresponding to the following amino acid substitutions: (a) R55A; (b) E117A; (c) N46A/E117A; (d) Q116A/R124A/R128A; (e) Q116A/R124D/R128A; (f) D43A/Q116A/R124A/R128A; (g) S45E/Q116A/R124A/R128A; or (h) Q48A/Q116A/R124A/R128A. In some embodiments, the polypeptides of the disclosure include an amino acid sequence having 100% sequence identity to SEQ ID NO: 1, and further include the amino acid substitutions corresponding to the following amino acid substitutions: (a) R55A; (b) E117A; (c) N46A/E117A; (d) Q116A/R124A/R128A; (e) Q116A/R124D/R128A; (f) D43A/Q116A/R124A/R128A; (g) S45E/Q116A/R124A/R128A; or (h) Q48A/Q116A/R124A/R128A.

In some embodiments, the recombinant polypeptides include an amino acid sequence having at least 70% sequence identity to the sequence of SEQ ID NO: 1, and further include an amino acid substitution corresponding an amino acid residue selected from the group consisting of D43H, D43R, S45R, S45G, Q49S, Q49G, Q116W, Q116K, R124Y, and R128K of SEQ ID NO: 1. In some embodiments, the recombinant polypeptides include an amino acid sequence having at least 75%, at least 80%, at least 85%, at least 90%, at least 95%, at least 96%, at least 97%, at least 98%, at least 99%, or at least 100% sequence identity to the sequence of SEQ ID NO: 1, and further include an amino acid substitution corresponding an amino acid residue selected from the group consisting of D43H, D43R, S45R, S45G, Q49S, Q49G, Q116W, Q116K, R124Y, and R128K of SEQ ID NO: 1. In some embodiments, the amino acid sequence of the recombinant polypeptides further include at least 1, at least 2, at least 3, at least 4, at least 5, at least 6, or at least 7 amino acid substitutions at a position corresponding to an amino acid residue selected from the group consisting of D43H, D43R, S45R, S45G, Q49S, Q49G, Q116W, Q116K, R124Y, and R128K of SEQ ID NO: 1.

In one aspect, provided herein are recombinant polypeptides including: (a) an amino acid sequence having at least 70% sequence identity to an interleukin 22 (IL-22) polypeptide having the amino acid sequence of SEQ ID NO: 6, and further including (b) one or more amino acid substitutions in the sequence of SEQ ID NO: 6. In some embodiments, the recombinant polypeptides include an amino acid sequence having at least 75%, at least 80%, at least 85%, at least 90%, at least 95%, at least 96%, at least 97%, at least 98%, or at least 99% to the sequence of SEQ ID NO: 6. In some embodiments, the recombinant polypeptides include an amino acid sequence having 100% sequence identify to the sequence of SEQ ID NO: 6.

In some embodiments, the amino acid sequence of the recombinant polypeptides disclosed herein further include one or more amino acid substitutions at a position corresponding to an amino acid residue selected from the group consisting of X43, X45, X46, X49, X116, X124, and X128 of SEQ ID NO: 6. In some embodiments, the amino acid sequence of the recombinant polypeptides further include 1 to 3, 2 to 5, 3 to 6, or 4 to 7 amino acid substitutions at a position corresponding to an amino acid residue selected from the group consisting of X43, X45, X46, X49, X116, X124, and X128 of SEQ ID NO: 6. In some embodiments, the amino acid sequence of the recombinant polypeptides further include at least 1, at least 2, at least 3, at least 4, at least 5, at least 6, or at least 7 amino acid substitutions at a position corresponding to an amino acid residue selected from the group consisting of X43, X45, X46, X49, X116, X124, and X128 of SEQ ID NO: 6.

In some embodiments, the amino acid sequence of the recombinant polypeptides further include one or more additional amino acid substitutions at a position corresponding to an amino acid residue selected from the group consisting of X48, X55, and X117 of SEQ ID NO: 6.

In some embodiments, the amino acid sequence of the recombinant polypeptides further include one additional amino acid substitution at a position corresponding to an amino acid residue selected from the group consisting of X48 of SEQ ID NO: 6. In some embodiments, the amino acid sequence of the recombinant polypeptides further include one additional amino acid substitution at a position corresponding to an amino acid residue selected from the group consisting of X55 of SEQ ID NO: 6. In some embodiments, the amino acid sequence of the recombinant polypeptides further include one additional amino acid substitution at a position corresponding to an amino acid residue selected from the group consisting of X117 of SEQ ID NO: 6.

In some embodiments, the amino acid substitution(s) is at a position corresponding to an amino acid residue selected from the group consisting of X43, X45, X46, X48, X55, and X117 of SEQ ID NO: 6. In some embodiments, the nucleic acids of the disclosure include a combination of amino acid substitutions at positions corresponding to amino acid residues X116, X124, X128 of SEQ ID NO: 6. In some embodiments, the amino acid sequence includes an amino acid substitution corresponding to amino acid residue X55 or X117 of SEQ ID NO: 1.

As discussed above, the IL-22 polypeptide variants disclosed herein can also include conservative modifications and substitutions at other positions of IL-22 (e.g., those that have a minimal effect on the secondary or tertiary structure of the IL-22 variants). Such conservative substitutions include those described by Dayhoff 1978, supra, and by Argos 1989, supra. For example, amino acids belonging to one of the following groups represent conservative changes: Group I: Ala, Pro, Gly, Gln, Asn, Ser, Thr; Group II: Cys, Ser, Tyr, Thr; Group III: Val, Ile, Leu, Met, Ala, Phe; Group IV: Lys, Arg, His; Group V: Phe, Tyr, Trp, His; and Group VI: Asp, Glu.

In some embodiments, the amino acid substitution(s) is independently selected from the group consisting of an alanine substitution, an arginine substitution, an aspartic acid substitution, a histidine substitution, a glutamic acid substitution, a lysine substitution, a serine substitution, a tryptophan substitution, and combinations of any thereof.

TABLE 2
Exemplary amino acid substitutions in the recombinant
IL-22 polypeptides of the disclosure.

| Position of | Original | Exemplary substitute amino |
|---|---|---|
| SEQ ID NO: 6 | amino acid | acid |
| 43 | D | A, R, N, Q, I, L, M, S, T, V, K, H |
| 45 | S | E, A, R, N, D, Q, E, I, L, K, M, V, G |
| 46 | N | A, R, D, E, I, K, K, M, S, V |
| 48 | Q | A, R, D, E, I, L, K, M, S, T, V |
| 49 | Q | A, R, D, E, I, L, K, M, S, T, V, G |
| 55 | R | A, N, D, Q, E, I, L, M, S, T, V |
| 116 | Q | A, R, D, E, I, L, K, M, S, T, V, W |
| 117 | E | A, R, N, Q, I, L, V, K, M, S, T |
| 124 | K | A, N, D, Q, E, I, L, M, S, T, V, Y |
| 128 | Q | A, N, D, R, K, E, I, L, M, S, T, V |

In some embodiments, the amino acid substitution(s) is at a position corresponding to an amino acid residue selected from the group consisting of E43, S45, N46, Q48, R55, Q116, E117, K124, Q128 of SEQ ID NO: 6. In some embodiments, the nucleic acids of the disclosure include an amino acid sequence having at least 70% sequence identity to SEQ ID NO: 6, and further including the amino acid substitutions corresponding to the following amino acid substitutions: (a) R55A; (b) E117A; (c) N46A/E117A; (d) Q116A/K124A/Q128A; (e) Q116A/K124D/Q128A; (f) E43A/Q116A/K124A/Q128A; (g) S45E/Q116A/K124A/Q128A; or (h) Q48A/Q116A/K124A/Q128A. In some embodiments, the polypeptides of the disclosure include an amino acid sequence having at least 75%, at least 80%, at least 85%, at least 90%, at least 95%, at least 96%, at least 97%, or at least 98%, at least 99% sequence identity to SEQ ID NO: 6, and further include the amino acid substitutions corresponding to the following amino acid substitutions: (a) R55A; (b) E117A; (c) N46A/E117A; (d) Q116A/K124A/Q128A; (e) Q116A/K124D/Q128A; (f) E43A/Q116A/K124A/Q128A; (g) S45E/Q116A/K124A/Q128A; or (h) Q48A/Q116A/K124A/Q128A. In some embodiments, the polypeptides of the disclosure include an amino acid sequence having 100% sequence identity to SEQ ID NO: 6, and further include the amino acid substitutions corresponding to the following amino acid substitutions: (a) R55A; (b) E117A; (c) N46A/E117A; (d) Q116A/K124A/Q128A; (e) Q116A/K124D/Q128A; (f) E43A/Q116A/K124A/Q128A; (g) S45E/Q116A/K124A/Q128A; or (h) Q48A/Q116A/K124A/Q128A.

In some embodiments, the nucleic acids of the disclosure include an amino acid sequence having at least 70% sequence identity to SEQ ID NO: 6, and further including an amino acid substitution corresponding an amino acid residue selected from the group consisting of E43H, E43R, S45R, S45G, Q49S, Q49G, Q116W, Q116K, Q124Y, and Q128K. In some embodiments, the recombinant polypeptides include an amino acid sequence having at least 75%, at least 80%, at least 85%, at least 90%, at least 95%, at least 96%, at least 97%, at least 98%, at least 99%, or at least 100% sequence identity to the sequence of SEQ ID NO: 1, and further include an amino acid substitution corresponding an amino acid residue selected from the group consisting of E43H, E43R, S45R, S45G, Q49S, Q49G, Q116W, Q116K, Q124Y, and Q128K of SEQ ID NO: 6. In some embodiments, the amino acid sequence of the recombinant polypeptides further include at least 1, at least 2, at least 3, at least 4, at least 5, at least 6, or at least 7 amino acid substitutions at a position corresponding to an amino acid residue selected from the group consisting of E43H, E43R, S45R, S45G, Q49S, Q49G, Q116W, Q116K, Q124Y, and Q128K of SEQ ID NO: 6. Exemplary IL-22 polypeptide variants according to this aspect can include substitutions of 1, 2, 3, 4, 5, 6, 7, 8, 9, 10 or more amino acids in the sequence of SEQ ID NO: 6.

In some embodiments, the amino acid substitution(s) in the sequence of the recombinant IL-22 polypeptide disclosed herein results in a modulation of binding affinity of the recombinant IL-22 polypeptide for IL10Rβ. The term “modulating”, in relation to the binding activity of an IL-22 polypeptide refers to a change in the binding affinity of the polypeptide for IL10Rβ. Modulation includes both increase (e.g., induce, stimulate) and decrease (e.g., reduce, inhibit), or otherwise affecting the binding affinity of the polypeptide. In some embodiments, the amino acid substitution(s) in the sequence of the recombinant IL-22 polypeptide disclosed herein reduces IL10Rβ-binding affinity of the recombinant IL-22 polypeptide compared to a reference IL-22 polypeptide lacking the amino acid substitution(s). In some embodiments, the amino acid substitution(s) increases IL10Rβ-binding affinity of the recombinant IL-22 polypeptide compared to a reference IL-22 polypeptide lacking the amino acid substitution(s).

The binding activity of recombinant polypeptides of the disclosure, including the IL-22 polypeptide variants described herein, can be assayed by any suitable method known in the art. For example, the binding activity of an IL-22 polypeptide variant disclosed herein and its receptors (e.g., IL-22R1 and/or IL10Rβ) can be determined by Scatchard analysis (Munsen et al. Analyt. Biochem. 107:220-239, 1980). Specific binding may also be assessed using techniques known in the art including but not limited to competition ELISA, Biacore® assays and/or KinExA® assays. A polypeptide that preferentially binds or specifically binds to a target protein is a concept well understood in the art, and methods to determine such specific or preferential binding are also known in the art.

A variety of assay formats may be used to select a recombinant IL-22 polypeptide that binds a ligand of interest (e.g., IL-22R1 and/or IL10Rβ). For example, solid-phase ELISA immunoassay, immunoprecipitation, Biacore™ (GE Healthcare, Piscataway, N.J.), KinExA, fluorescence-activated cell sorting (FACS), Octet™ (ForteBio, Inc., Menlo Park, Calif.) and Western blot analysis are among many assays that may be used to identify a polypeptide that specifically reacts with a receptor or a ligand binding portion thereof, that specifically binds with a cognate ligand or binding partner. Generally, a specific or selective binding reaction will be at least twice the background signal or noise, more typically more than 10 times background, more than 20 times background, even more typically, more than 50 times background, more than 75 times background, more than 100 times background, yet more typically, more than 500 times background, even more typically, more than 1000 times background, and even more typically, more than 10,000 times background.

One of ordinary skill in the art will appreciate that binding affinity can also be used as a measure of the strength of a non-covalent interaction between two molecules, e.g., an IL-22 polypeptide and an IL10Rβ receptor. In some instance, binding affinity is used to describe monovalent interactions (intrinsic activity). Binding affinity between two molecules may be quantified by determination of the dissociation constant (K_{D}). In turn, K_{D} can be determined by measurement of the kinetics of complex formation and dissociation using, e.g., the surface plasmon resonance (SPR) method (Biacore). The rate constants corresponding to the association and the dissociation of a monovalent complex are referred to as the association rate constants kₐ (or kₒₙ) and dissociation rate constant k_{d} (or k_{off}), respectively. K_{D} is related to kₐ and k_{d} through the equation K_{D}=kd/kₐ. The value of the dissociation constant can be determined directly by well-known methods and can be computed even for complex mixtures by methods such as those set forth in Caceci et al. (Byte 9: 340-362, 1984). For example, the K_{D} may be established using a double-filter nitrocellulose filter binding assay such as that disclosed by Wong & Lohman (1993, Proc. Natl. Acad. Sci. USA 90: 5428-5432). Other standard assays to evaluate the binding ability of the IL-22 polypeptide variants of the present disclosure towards target receptors are known in the art, including for example, ELISAs, Western blots, RIAs, and flow cytometry analysis, and other assays exemplified in the Examples. The binding kinetics and binding affinity of the IL-22 polypeptide variants also can be assessed by standard assays known in the art, such as Surface Plasmon Resonance (SPR), e.g. by using a Biacore™ system, or KinExA. In some embodiments, the binding affinity of the IL-22 polypeptide variant of the disclosure to IL-22R1 and/or IL10Rβ is determined by a solid-phase receptor binding assay (Matrosovich M N et al., Methods Mol Biol. 865:71-94, 2012). In some embodiments, the binding affinity of the Il-22 polypeptide variant of the disclosure to IL-22R1 and/or IL10Rβ is determined by a Surface Plasmon Resonance (SPR) assay.

In some embodiments, the amino acid substitutions in the recombinant IL-22 polypeptide variants disclosed herein result in a tissue-selective IL-22 signaling compared to a reference IL-22 polypeptide lacking the amino acid substitutions. As described in further detail below, an exemplary IL-22 polypeptide variant of the disclosure, 22-B3, elicites tissue-selective signaling responses in vivo, acting as a STAT3-biased agonist in the pancreas and colon but a neutral antagonist in the skin and liver. In particular, 22-B3 variant retains the tissue protective functions of IL-22 in vivo without inducing inflammatory mediators in the skin, liver, and colon, thereby uncoupling the major tissue protective and pro-inflammatory functions of IL-22. Accordingly, in some embodiments, the amino acid substitutions in the recombinant IL-22 polypeptide variants disclosed herein result in a tissue-selective IL-22 signaling which involves a reduction of IL-22 signaling in the skin while substantially retains IL-22 signaling in the pancreas and/or the gastrointestinal tract. In some other embodiments, the amino acid substitutions in the recombinant IL-22 polypeptide variants disclosed herein result in a tissue-selective IL-22 signaling which involves a reduction of IL-22 signaling in the liver while substantially retains IL-22 signaling in the pancreas and/or the gastrointestinal tract.

In some embodiments, the amino acid substitution(s) in the IL-22 polypeptide variants disclosed herein results in a biased IL-22 signaling as determined by, for example, phosphorylation of STAT1 and STAT3, compared to a reference IL-22 polypeptide lacking the amino acid substitution(s). In some embodiments, the biased IL-22 signaling includes a reduction in a STAT1 phosphorylation by at least 10%, e.g., by at least 10%, at least 20%, at least 30%, at least 40%, at least 50%, at least 60%, at least 70%, at least 80%, or at least 90% when compared to a reference IL-22 polypeptide lacking the amino acid substitution(s). In some embodiments, the biased IL-22 signaling includes a reduction in a STAT3 phosphorylation by at least 10%, e.g., by at least 10%, at least 20%, at least 30%, at least 40%, at least 50%, at least 60%, at least 70%, at least 80%, or at least 90% when compared to a reference IL-22 polypeptide lacking the amino acid substitution(s).

In some embodiments, the amino acid substitution(s) in the IL-22 polypeptide variants disclosed herein results in a reduction in one or more STAT1-mediated pro-inflammatory functions. There are no specific limitations to the STAT1-mediated functions that can be suitably assayed. Non-limiting examples of suitable STAT1-mediated pro-inflammatory function include cytokine production, chemokine production, and immune cell recruitment. In some embodiments, the STAT1-mediated pro-inflammatory function is reduced about 20% to about 100%. In some embodiments, the STAT1 signaling is determined by an assay selected from the group consisting of by a gene expression assay, a phospho-flow signaling assay, and an enzyme-linked immunosorbent assay (ELISA). In some embodiments, the amino acid substitution(s) in the IL-22 polypeptide variants disclosed herein results in a reduction in one or more STAT1-mediated pro-inflammatory functions, as determined by the polypeptides' capacity to induce expression of a pro-inflammatory gene. Non-limiting examples of pro-inflammatory genes include CXCL1, CXCL2, CXCL8, CXCL9, CXCL10, IL-1β, and IL-6. In some embodiments, the STAT1-mediated pro-inflammatory function is reduced about 20% to about 100%, for example, about 20% to about 50%, about 30% to about 60%, about 40% to about 70%, about 50% to about 80%, about 40% to about 90%, bout 50% to about 100%, about 40% to about 80%, about 30% to about 70%, about 20% to about 80%, about 20% to about 70%, about 20% to about 60%, about 30% to about 80%, about 30% to about 90%, or about 30% to about 100% compared to a reference IL-20 lacking the amino acid substitution(s).

In some embodiments, the amino acid substitution(s) in the IL-22 polypeptide variants disclosed herein substantially retain one or more STAT3-mediated function. There are no specific restrictions with respect to the STAT3-mediated functions that can be suitably evaluated. Examples of suitable STAT3-mediated functions include, but are not limited to tissue protection, tissue regeneration, cell proliferation, and cell survival. In some embodiments, the amino acid substitution(s) in the IL-22 polypeptide variants disclosed herein substantially retains one or more STAT3-mediated function, as determined by the polypeptides' capacity to induce expression of a biomarker, such as Reg3β, Reg3γ, Muc1, Muc2, Muc10, BCL-2, Cyclin-D, Claudin-2, LCN2, or β-Defensin.

In some embodiments, the amino acid substitution(s) in the IL-22 polypeptide variants disclosed herein results in a reduction in a STAT1-mediated pro-inflammatory function while substantially retains its STAT3-mediated function. In some embodiments, the biased IL-22 signaling includes a ratio of STAT1-mediated signaling to STAT3-mediated signaling ranging from about 1:1.5 to about 1:10 such as, for example, about 1:2 to about 1:5, about 1:2 to about 1:7, about 1:3 to about 1:8, about 1:4 to about 1:10, about 1:4 to about 1:9, or about 1:4 to about 1:8. In some embodiments, the biased IL-22 signaling includes a ratio of STAT1-mediated signaling to STAT3-mediated signaling ranging from about 1.5 to 3.2. In some embodiments, the STAT1 signaling and/or STAT3 signaling is determined by an assay selected from the group consisting of by a gene expression assay, a phospho-flow signaling assay, and an enzyme-linked immunosorbent assay (ELISA).

### B. Nucleic Acids

In one aspect, provided herein are various nucleic acid molecules including nucleotide sequences encoding the recombinant IL-22 polypeptides the disclosure, including expression cassettes, and expression vectors containing these nucleic acid molecules operably linked to heterologous nucleic acid sequences such as, for example, regulator sequences which allow in vivo expression of the recombinant IL-22 polypeptide in a host cell or ex-vivo cell-free expression system.

The terms “nucleic acid molecule” and “polynucleotide” are used interchangeably herein, and refer to both RNA and DNA molecules, including nucleic acid molecules comprising cDNA, genomic DNA, synthetic DNA, and DNA or RNA molecules containing nucleic acid analogs. A nucleic acid molecule can be double-stranded or single-stranded (e.g., a sense strand or an antisense strand). A nucleic acid molecule may contain unconventional or modified nucleotides. The terms “polynucleotide sequence” and “nucleic acid sequence” as used herein interchangeably refer to the sequence of a polynucleotide molecule. The polynucleotide and polypeptide sequences disclosed herein are shown using standard letter abbreviations for nucleotide bases and amino acids as set forth in 37 CFR § 1.82), which incorporates by reference WIPO Standard ST.25 (1998), Appendix 2, Tables 1-6.

Nucleic acid molecules of the present disclosure can be nucleic acid molecules of any length, including nucleic acid molecules that are generally between about 0.5 Kb and about 20 Kb, for example between about 0.5 Kb and about 20 Kb, between about 1 Kb and about 15 Kb, between about 2 Kb and about 10 Kb, or between about 5 Kb and about 25 Kb, for example between about 10 Kb to 15 Kb, between about 15 Kb and about 20 Kb, between about 5 Kb and about 20 Kb, about 5 Kb and about 10 Kb, or about 10 Kb and about 25 Kb.

In some embodiments disclosed herein, the nucleic acid molecules of the disclosure include a nucleotide sequence encoding a polypeptide which includes an amino acid sequence having at least 90%, at least 95%, at least 96%, at least 97, at least 98%, at least 99%, or at least 100% sequence identity to the amino acid sequence of a recombinant polypeptide as disclosed herein. In some embodiments, the nucleic acid molecules of the disclosure include a nucleotide sequence encoding a polypeptide that includes: (a) an amino acid sequence having at least 70%, 80%, 90%, 95%, 99%, or 100% sequence identity to an IL-22 polypeptide having the amino acid sequence of SEQ ID NO: 1; and further including (b) one or more amino acid substitution at a position corresponding to an amino acid residue selected from the group consisting of X43, X49, X45, X46, X116, X124, and X128 of SEQ ID NO: 1. In some embodiments, the polypeptide further includes an additional amino acid substitution at a position corresponding to an amino acid residue selected from the group consisting of X48, X55, and X117 of SEQ ID NO: 1. In some embodiments, the amino acid substitution(s) is at a position corresponding to an amino acid residue selected from the group consisting of X43, X45, X46, X48, X49, X55, and X117 of SEQ ID NO: 1. In some embodiments, the polypeptides of the disclosure further include a combination of amino acid substitutions at positions corresponding to amino acid residues X116, X124, X128 of SEQ ID NO: 1. In some embodiments, the amino acid sequence includes an amino acid substitution corresponding to amino acid residue X55 or X117 of SEQ ID NO: 1. In some embodiments, the nucleic acid molecules of the disclosure include a nucleotide sequence encoding a polypeptide that includes an amino acid sequence having at least 70%, 80%, 90%, 95%, 99%, or 100% sequence identity to SEQ ID NO: 1, and further include an amino acid substitution corresponding an amino acid residue selected from the group consisting of D43H, D43R, S45R, S45G, Q49S, Q49G, Q116W, Q116K, R124Y, and R128K of SEQ ID NO: 1. In some embodiments, the nucleic acid molecules of the disclosure include a nucleotide sequence encoding a polypeptide that includes an amino acid sequence having at least 70% sequence identity to SEQ ID NO: 1, and further include the amino acid substitutions corresponding to the following amino acid substitutions: (a) R55A; (b) E117A; (c) N46A/E117A; (d) Q116A/R124A/R128A; (e) Q116A/R124D/R128A; (f) D43A/Q116A/R124A/R128A; (g) S45E/Q116A/R124A/R128A; or (h) Q48A/Q116A/R124A/R128A.

In some embodiments, the nucleic acid molecules of the disclosure include a nucleotide sequence encoding a polypeptide that includes: (a) an amino acid sequence having at least 70%, 80%, 90%, 95%, 99%, or 100% sequence identity to an IL-22 polypeptide having the amino acid sequence of SEQ ID NO: 6; and further including (b) one or more amino acid substitution at a position corresponding to an amino acid residue selected from the group consisting of X43, X45, X46, X49, X116, X124, and X128 of SEQ ID NO: 6. In some embodiments, the polypeptide further includes an additional amino acid substitution at a position corresponding to an amino acid residue selected from the group consisting of X48, X55, and X117 of SEQ ID NO: 6. In some embodiments, the amino acid substitution(s) is at a position corresponding to an amino acid residue selected from the group consisting of X43, X45, X46, X48, X55, and X117 of SEQ ID NO: 6. In some embodiments, the polypeptides of the disclosure further include a combination of amino acid substitutions at positions corresponding to amino acid residues X116, X124, X128 of SEQ ID NO: 6. In some embodiments, the amino acid sequence includes an amino acid substitution corresponding to amino acid residue X55 or X117 of SEQ ID NO: 6. In some embodiments, the nucleic acid molecules of the disclosure include a nucleotide sequence encoding a polypeptide that includes an amino acid sequence having at least 70%, 80%, 90%, 95%, 99%, or 100% sequence identity to SEQ ID NO: 6, and further include an amino acid substitution corresponding an amino acid residue selected from the group consisting of E43H, E43R, S45R, S45G, Q49S, Q49G, Q116W, Q116K, Q124Y, and Q128K of SEQ ID NO: 6. In some embodiments, the nucleic acid molecules of the disclosure include a nucleotide sequence encoding a polypeptide that includes an amino acid sequence having at least 70% sequence identity to SEQ ID NO: 6, and further include the amino acid substitutions corresponding to the following amino acid substitutions: (a) R55A; (b) E117A; (c) N46A/E117A; (d) Q116A/K124A/Q128A; (e) Q116A/K124D/Q128A; (f) E43A/Q116A/K124A/Q128A; (g) S45E/Q116A/K124A/Q128A; or (h) Q48A/Q116A/K124A/Q128A.

In some embodiments disclosed herein, the nucleic acid molecules of the disclosure include a nucleotide sequence encoding a polypeptide which includes an amino acid sequence having at least 90%, 95%, 96%, 97, 98%, 99% sequence identity to an amino acid sequence selected from the group consisting of SEQ ID NOS: 1-5 and 7-14. In some embodiments, the nucleic acid molecules of the disclosure include a nucleotide sequence encoding a polypeptide which includes an amino acid sequence having at least 90%, 95%, 96%, 97, 98%, 99%, or 100% sequence identity to the amino acid sequence of SEQ ID NO: 2. In some embodiments, the nucleic acid molecules of the disclosure include a nucleotide sequence encoding a polypeptide which includes an amino acid sequence having at least 90%, 95%, 96%, 97, 98%, 99%, or 100% sequence identity to the amino acid sequence of SEQ ID NO: 3. In some embodiments, the nucleic acid molecules of the disclosure include a nucleotide sequence encoding a polypeptide which includes an amino acid sequence having 90%, 95%, 96%, 97, 98%, 99%, or 100% sequence identity to the amino acid sequence of SEQ ID NO: 4. In some embodiments, the nucleic acid molecules of the disclosure include a nucleotide sequence encoding a polypeptide which includes an amino acid sequence having 90%, 95%, 96%, 97, 98%, 99%, or 100% sequence identity to the amino acid sequence of SEQ ID NO: 5. In some embodiments, the nucleic acid molecules of the disclosure include a nucleotide sequence encoding a polypeptide which includes an amino acid sequence selected from the group consisting of SEQ ID NOS: 7-14. In some embodiments, the nucleic acid molecules of the disclosure include a nucleotide sequence encoding a polypeptide which includes an amino acid sequence having 90%, 95%, 96%, 97, 98%, 99%, or 100% sequence identity to the amino acid sequence of SEQ ID NO: 11.

In some embodiments, the nucleotide sequence is incorporated into an expression cassette or an expression vector. It will be understood that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell and/or into an individual. As such, in some embodiments, an expression cassette of the disclosure include a coding sequence for the recombinant polypeptide as disclosed herein, which is operably linked to expression control elements, such as a promoter, and optionally, any or a combination of other nucleic acid sequences that affect the transcription or translation of the coding sequence.

In some embodiments, the nucleotide sequence is incorporated into an expression vector. It will be understood by one skilled in the art that the term “vector” generally refers to a recombinant polynucleotide construct designed for transfer between host cells, and that may be used for the purpose of transformation, e.g., the introduction of heterologous DNA into a host cell. As such, in some embodiments, the vector can be a replicon, such as a plasmid, phage, or cosmid, into which another DNA segment may be inserted so as to bring about the replication of the inserted segment. In some embodiments, the expression vector can be an integrating vector.

In some embodiments, the expression vector can be a viral vector. As will be appreciated by one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. In some embodiments, the viral vector is an adenoviral vector, AAV vector, bacculorival vector, a retroviral vector, or a lentiviral vector. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus, which is a genus of retrovirus.

Accordingly, also provided herein are vectors, plasmids, or viruses containing one or more of the nucleic acid molecules encoding any recombinant polypeptide or IL-22 polypeptide variant disclosed herein. The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan.

DNA vectors can be introduced into eukaryotic cells via conventional transformation or transfection techniques. Suitable methods for transforming or transfecting host cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection.

Viral vectors that can be used in the disclosure include, for example, baculoviral vectors, retrovirus vectors, adenovirus vectors, and adeno-associated virus vectors, lentivirus vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

The precise components of the expression system are not critical. For example, a recombinant polypeptide as disclosed herein can be produced in a eukaryotic host, such as a mammalian cells (e.g., COS cells, NIH 3T3 cells, or HeLa cells). These cells are available from many sources, including the American Type Culture Collection (Manassas, Va.). In selecting an expression system, care should be taken to ensure that the components are compatible with one another. Artisans or ordinary skill are able to make such a determination. Furthermore, if guidance is required in selecting an expression system, skilled artisans may consult P. Jones, “Vectors: Cloning Applications”, John Wiley and Sons, New York, N.Y., 2009).

The nucleic acid molecules provided can contain naturally occurring sequences, or sequences that differ from those that occur naturally, but, due to the degeneracy of the genetic code, encode the same polypeptide, e.g., antibody. These nucleic acid molecules can consist of RNA or DNA (for example, genomic DNA, cDNA, or synthetic DNA, such as that produced by phosphoamidite-based synthesis), or combinations or modifications of the nucleotides within these types of nucleic acids. In addition, the nucleic acid molecules can be double-stranded or single-stranded (e.g., either a sense or an antisense strand).

The nucleic acid molecules are not limited to sequences that encode polypeptides (e.g., IL-22 polypeptide variants); some or all of the non-coding sequences that lie upstream or downstream from a coding sequence (e.g., the coding sequence of an IL-22 polypeptide variant) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

In another aspect, provided herein are cell cultures including at least one recombinant cell as disclosed herein, and a culture medium. Generally, the culture medium can be any suitable culture medium for culturing the cells described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### C. Recombinant Cells and Cell Cultures

The recombinant nucleic acids of the present disclosure can be introduced into a host cell, such as, for example, a human T lymphocyte, to produce a recombinant cell containing the nucleic acid molecule. Introduction of the nucleic acid molecules of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid molecules can be delivered by viral or non-viral delivery vehicles known in the art. For example, the nucleic acid molecule can be stably integrated in the host genome, or can be episomally replicating, or present in the recombinant host cell as a mini-circle expression vector for transient expression. Accordingly, in some embodiments, the nucleic acid molecule is maintained and replicated in the recombinant host cell as an episomal unit. In some embodiments, the nucleic acid molecule is stably integrated into the genome of the recombinant cell. Stable integration can be achieved using classical random genomic recombination techniques or with more precise techniques such as guide RNA-directed CRISPR/Cas9 genome editing, or DNA-guided endonuclease genome editing with NgAgo (Natronobacterium gregoryi Argonaute), or TALENs genome editing (transcription activator-like effector nucleases). In some embodiments, the nucleic acid molecule is present in the recombinant host cell as a mini-circle expression vector for transient expression.

The nucleic acid molecules can be encapsulated in a viral capsid or a lipid nanoparticle, or can be delivered by viral or non-viral delivery means and methods known in the art, such as electroporation. For example, introduction of nucleic acids into cells may be achieved by viral transduction. In a non-limiting example, baculoviral virus or adeno-associated virus (AAV) can be engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

In some embodiments, host cells can be genetically engineered (e.g., transduced or transformed or transfected) with, for example, a vector construct of the present application that can be, for example, a viral vector or a vector for homologous recombination that includes nucleic acid sequences homologous to a portion of the genome of the host cell, or can be an expression vector for the expression of the polypeptides of interest. Host cells can be either untransformed cells or cells that have already been transfected with at least one nucleic acid molecule.

In some embodiments, the recombinant cell is a prokaryotic cell or a eukaryotic cell. In some embodiments, the cell is in vivo. In some embodiments, the cell is ex vivo. In some embodiments, the cell is in vitro. In some embodiments, the recombinant cell is a eukaryotic cell. In some embodiments, the recombinant cell is an animal cell. In some embodiments, the animal cell is a mammalian cell. In some embodiments, the animal cell is a human cell. In some embodiments, the cell is a non-human primate cell. In some embodiments, the recombinant cell is an immune system cell, e.g., a lymphocyte (e.g., a T cell or NK cell), or a dendritic cell. In some embodiments, the immune cell is a B cell, a monocyte, a natural killer (NK) cell, a basophil, an eosinophil, a neutrophil, a dendritic cell, a macrophage, a regulatory T cell, a helper T cell (T_{H}), a cytotoxic T cell (T_{CTL}), or other T cell. In some embodiments, the immune system cell is a T lymphocyte. In some embodiments, the cell can be obtained by leukapheresis performed on a sample obtained from a subject. In some embodiments, the subject is a human patient. Non-limiting examples of suitable cell lines include Trichoplusia ni (Hi5) cells, Expi-293F cells, HEK-293T (ATCC CRL-3216), HT-29 (ATCC HTB-38), Panc-1 (ATCC CRL-1469), HepG2 (ATCC HB-8065), and EC4 cells.

In another aspect, provided herein are cell cultures including at least one recombinant cell as disclosed herein, and a culture medium. Generally, the culture medium can be any suitable culture medium for culturing the cells described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### D. Methods for Producing an IL-22 Polypeptide

In another aspect, some embodiments of the disclosure relate to various methods for producing a recombinant polypeptide of the disclosure, the methods include: (a) providing one or more recombinant cells of the disclosure; and culturing the recombinant cell(s) in a culture medium such that the cells produce the polypeptide encoded by the recombinant nucleic acid molecule. Accordingly, the recombinant polypeptides produced by the method disclosed herein are also within the scope of the disclosure.

Non-limiting exemplary embodiments of the disclosed methods for producing a recombinant polypeptide can include one or more of the following features. In some embodiments, the methods further include isolating and/or purifying the produced polypeptide. In some embodiments, the methods for producing a recombinant polypeptide of the disclosure further include isolating and/or purifying the produced polypeptide. In some embodiments, the methods for producing a polypeptide of the disclosure further include structurally modifying the produced polypeptide to increase half-life.

In some embodiments, the modification includes one or more alterations selected from the group consisting of fusion to a human Fc antibody fragment, fusion to albumin, and PEGylation. For example, any of the recombinant polypeptides disclosed herein can be prepared as fusions or chimeric polypeptides that include a recombinant polypeptide and a heterologous polypeptide (e.g., a polypeptide that is not IL-22 or a variant thereof). Exemplary heterologous polypeptides can increase the circulating half-life of the recombinant polypeptide in vivo, and may, therefore, further enhance the properties of the recombinant polypeptides of the disclosure. In various embodiments, the heterologous polypeptide that increases the circulating half-life may be a serum albumin, such as human serum albumin, or the Fc region of the IgG subclass of antibodies that lacks the IgG heavy chain variable region. Exemplary Fc regions can include a mutation that inhibits complement fixation and Fc receptor binding, or it may be lytic, e.g., able to bind complement or to lyse cells via another mechanism, such as antibody-dependent complement lysis (ADCC).

In some embodiments, the “Fc region” can be a naturally occurring or synthetic polypeptide that is homologous to the IgG C-terminal domain produced by digestion of IgG with papain. IgG Fc has a molecular weight of approximately 50 kDa. The recombinant fusion polypeptides of the disclosure can include the entire Fc region, or a smaller portion thereof that retains the ability to extend the circulating half-life of a fusion polypeptide of which it is a part. In addition, full-length or fragmented Fc regions can be variants of the wild-type molecule. That is, they can contain mutations that may or may not affect the function of the polypeptides; as described further below, native activity is not necessary or desired in all cases. In some embodiments, the recombinant fusion protein (e.g., an IL-22 partial agonist or antagonist as described herein) includes an IgG1, IgG2, IgG3, or IgG4 Fc region.

The Fc region can be “lytic” or “non-lytic”, but is typically non-lytic. A non-lytic Fc region typically lacks a high affinity Fc receptor binding site and a C′lq binding site. The high affinity Fc receptor binding site of murine IgG Fc includes the Leu residue at position 235 of IgG Fc. Thus, the Fc receptor binding site can be destroyed by mutating or deleting Leu 235. For example, substitution of Glu for Leu 235 inhibits the ability of the Fc region to bind the high affinity Fc receptor. The murine C′lq binding site can be functionally destroyed by mutating or deleting the Glu 318, Lys 320, and Lys 322 residues of IgG. For example, substitution of Ala residues for Glu 318, Lys 320, and Lys 322 renders IgG1 Fc unable to direct antibody-dependent complement lysis. In contrast, a lytic IgG Fc region has a high affinity Fe receptor binding site and a C′lq binding site. The high affinity Fc receptor binding site includes the Leu residue at position 235 of IgG Fc, and the C′lq binding site includes the Glu 318, Lys 320, and Lys 322 residues of IgG1. Lytic IgG Fc has wild-type residues or conservative amino acid substitutions at these sites. Lytic IgG Fc can target cells for antibody dependent cellular cytotoxicity or complement directed cytolysis (CDC). Appropriate mutations for human IgG are also known in the art.

In other embodiments, the recombinant fusion polypeptide can include a recombinant IL-22 polypeptide of the disclosure and a polypeptide that functions as an antigenic tag, such as a FLAG sequence. FLAG sequences are recognized by biotinylated, highly specific, anti-FLAG antibodies. In some embodiments, the recombinant fusion polypeptide further includes a C-terminal c-myc epitope tag.

In other embodiments, the recombinant fusion polypeptide includes a recombinant IL-22 polypeptide of the disclosure and a heterologous polypeptide that functions to enhance expression or direct cellular localization of the IL-22 polypeptide, such as the Aga2p agglutinin subunit.

In other embodiments, a fusion polypeptide including a recombinant IL-22 polypeptide of the disclosure and an antibody or antigen-binding portion thereof can be generated. The antibody or antigen-binding component of the recombinant IL-22 polypeptide can serve as a targeting moiety. For example, it can be used to localize the recombinant IL-22 polypeptide to a particular subset of cells or target molecule. Methods of generating cytokine-antibody chimeric polypeptides are known in the art.

In some embodiments, the recombinant IL-22 polypeptides of the disclosure can be modified with one or more polyethylene glycol (PEG) molecules to increase its half-life. The term “PEG” as used herein means a polyethylene glycol molecule. In its typical form, PEG is a linear polymer with terminal hydroxyl groups and has the formula HO—CH₂CH₂—(CH₂CH₂O)n-CH₂CH₂—OH, where n is from about 8 to about 4000.

Generally, “n” is not a discrete value but constitutes a range with approximately Gaussian distribution around an average value. The terminal hydrogen may be substituted with a capping group such as an alkyl or alkanol group. PEG can have at least one hydroxy group, more preferably it is a terminal hydroxy group. This hydroxy group is can be attached to a linker moiety which can react with the peptide to form a covalent linkage. Numerous derivatives of PEG exist in the art. The PEG molecule covalently attached to the recombinant IL-22 polypeptides of the present disclosure may be approximately 10,000, 20,000, 30,000, or 40,000 daltons average molecular weight. PEGylation reagents may be linear or branched molecules and may be present singularly or in tandem. The PEGylated IL-22 polypeptides of the present disclosure can have tandem PEG molecules attached to the C-terminus and/or the N-terminus of the peptide. The term “PEGylation” as used herein means the covalent attachment of one or more PEG molecules, as described above, to a molecule such as the IL-22 polypeptides of the present disclosure.

### E. Pharmaceutical Compositions

The recombinant polypeptides, nucleic acids, recombinant cells, and/or cell cultures of the disclosure can be incorporated into compositions, including pharmaceutical compositions. Such compositions generally include the recombinant polypeptides, nucleic acids, recombinant cells, and/or cell cultures as described herein and a pharmaceutically acceptable excipient, e.g., carrier.

Accordingly, one aspect of the present disclosure relates to pharmaceutical compositions that include a pharmaceutical acceptable carrier and one or more of the following: (a) a recombinant polypeptide of the disclosure; (b) a recombinant nucleic acid of the disclosure; (c) a recombinant cell of the disclosure; and (d) a pharmaceutically acceptable carrier.

In some embodiments, the pharmaceutical compositions of the disclosure are formulated for the treating, preventing, ameliorating a disease such as cancer, or for reducing or delaying the onset of the disease.

Non-limiting exemplary embodiments of the disclosed pharmaceutical compositions can include one or more of the following features. In some embodiments, the composition includes a recombinant polypeptide of the disclosure and a pharmaceutically acceptable carrier. In some embodiments, the composition includes a recombinant cell of the disclosure and a pharmaceutically acceptable carrier. In some embodiments, the recombinant cell expresses a recombinant polypeptide of the disclosure. Examples of recombinant cells genetically modified to express and secret interleukin as new therapeutic approaches are described previously in, for example, Steidler L. et al., Nature Biotechnology, Vol. 21, No. 7, July 2003 and Oh J. H et al., mSphere, Vol. 5, Issue 3, May/June 2020.

In some embodiments, the composition includes a recombinant nucleic acid of the disclosure and a pharmaceutically acceptable carrier. In some embodiments, the recombinant nucleic acid is encapsulated in a viral capsid or a lipid nanoparticle.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM. (BASF, Parsippany, N.J.), or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Systemic administration of the subject recombinant polypeptides of the disclosure can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art

In some embodiments, the recombinant polypeptides of the disclosure can also be administered by transfection or infection using methods known in the art, including but not limited to the methods described in McCaffrey et al. (Nature 418:6893, 2002), Xia et al. (Nature Biotechnol. 20: 1006-1010, 2002), or Putnam (Am. J. Health Syst. Pharm. 53: 151-160, 1996, erratum at Am. J. Health Syst. Pharm. 53:325, 1996).

In some embodiments, the subject recombinant polypeptides of the disclosure are prepared with carriers that will protect the recombinant polypeptides against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.As described in greater detail below, the recombinant polypeptides of the present disclosure may also be modified to achieve extended duration of action such as by PEGylation, acylation, Fc fusions, linkage to molecules such as albumin, etc. In some embodiments, the recombinant polypeptides can be further modified to prolong their half-life in vivo and/or ex vivo. Non-limiting examples of known strategies and methodologies suitable for modifying the recombinant polypeptides of the disclosure include (1) chemical modification of a recombinant polypeptide described herein with highly soluble macromolecules such as polyethylene glycol (“PEG”) which prevents the recombinant polypeptides from contacting with proteases; and (2) covalently linking or conjugating a recombinant polypeptide described herein with a stable protein such as, for example, albumin. Accordingly, in some embodiments, the recombinant polypeptides of the disclosure can be fused to a stable protein, such as, albumin. For example, human albumin is known as one of the most effective proteins for enhancing the stability of polypeptides fused thereto and there are many such fusion proteins reported.

In some embodiments, the pharmaceutical compositions of the disclosure include one or more pegylation reagents. As used herein, the term “PEGylation” refers to modifying a protein by covalently attaching polyethylene glycol (PEG) to the protein, with “PEGylated” referring to a protein having a PEG attached. A range of PEG, or PEG derivative sizes with optional ranges of from about 10,000 Daltons to about 40,000 Daltons may be attached to the recombinant polypeptides of the disclosure using a variety of chemistries. In some embodiments, the average molecular weight of said PEG, or PEG derivative, is about 1 kD to about 200 kD such as, e.g., about 10 kD to about 150 kD, about 50 kD to about 100 kD, about 5 kD to about 100 kD, about 20 kD to about 80 kD, about 30 kD to about 70 kD, about 40 kD to about 60 kD, about 50 kD to about 100 kD, about 100 kD to about 200 kD, or about 150 kD to about 200 kD. In some embodiments, the average molecular weight of said PEG, or PEG derivative, is about 5 kD, about 10 kD, about 20 kD, about 30 kD, about 40 kD, about 50 kD, about 60 kD, about 70 kD, or about 80 kD. In some embodiments, the average molecular weight of said PEG, or PEG derivative, is about 40 kD. In some embodiments, the pegylation reagent is selected from methoxy polyethylene glycol-succinimidyl propionate (mPEG-SPA), mPEG-succinimidyl butyrate (mPEG-SBA), mPEG-succinimidyl succinate (mPEG-SS), mPEG-succinimidyl carbonate (mPEG-SC), mPEG-Succinimidyl Glutarate (mPEG-SG), mPEG-N-hydroxyl-succinimide (mPEG-NHS), mPEG-tresylate and mPEG-aldehyde. In some embodiments, the pegylation reagent is polyethylene glycol; for example said pegylation reagent is polyethylene glycol with an average molecular weight of 20,000 Daltons covalently bound to the N-terminal methionine residue of the recombinant polypeptides of the disclosure. In some embodiments, the pegylation reagent is polyethylene glycol with an average molecular weight of about 5 kD, about 10 kD, about 20 kD, about 30 kD, about 40 kD, about 50 kD, about 60 kD, about 70 kD, or about 80 kD covalently bound to the N-terminal methionine residue of the recombinant polypeptides of the disclosure. In some embodiments, the pegylation reagent is polyethylene glycol with an average molecular weight of about 40 kD covalently bound to the N-terminal methionine residue of the recombinant polypeptides of the disclosure.

Accordingly, in some embodiments, the recombinant polypeptides of the disclosure are chemically modified with one or more polyethylene glycol moieties, e.g., PEGylated; or with similar modifications, e.g. PASylated. In some embodiments, the PEG molecule or PAS molecule is conjugated to one or more amino acid side chains of the disclosed recombinant polypeptide. In some embodiments, the PEGylated or PASylated polypeptide contains a PEG or PAS moiety on only one amino acid. In other embodiments, the PEGylated or PASylated polypeptide contains a PEG or PAS moiety on two or more amino acids, e.g., attached to two or more, five or more, ten or more, fifteen or more, or twenty or more different amino acid residues. In some embodiments, the PEG or PAS chain is 2000, greater than 2000, 5000, greater than 5,000, 10,000, greater than 10,000, greater than 10,000, 20,000, greater than 20,000, and 30,000 Da. The PASylated polypeptide may be coupled directly to PEG or PAS (e.g., without a linking group) through an amino group, a sulfhydryl group, a hydroxyl group, or a carboxyl group. In some embodiments, the recombinant polypeptide of the disclosure is covalently bound to a polyethylene glycol with an average molecular weight ranging from about 1 kD to about 200 kD such as, e.g., about 10 kD to about 150 kD, about 50 kD to about 100 kD, about 5 kD to about 100 kD, about 20 kD to about 80 kD, about 30 kD to about 70 kD, about 40 kD to about 60 kD, about 50 kD to about 100 kD, about 100 kD to about 200 kD, or about 150 kD to about 200 kD. In some embodiments, the recombinant polypeptide of the disclosure is covalently bound to a polyethylene glycol with an average molecular weight of about 5 kD, about 10 kD, about 20 kD, about 30 kD, about 40 kD, about 50 kD, about 60 kD, about 70 kD, or about 80 kD. In some embodiments, the recombinant polypeptide of the disclosure is covalently bound to a polyethylene glycol with an average molecular weight of about 40 kD.

### Incorporation of Site-Specific PEGylation Sites

In some embodiments, the recombinant polypeptides of the disclosure (e.g., IL-22 variants) may be modified by the incorporation of non-natural amino acids with non-naturally occurring amino acid side chains to facilitate site specific conjugation (e.g., PEGylation) as described in, for example, U.S. Pat. Nos. 7,045,337; 7,915,025; Dieters, et al. (2004) Bioorganic and Medicinal Chemistry Letters 14(23):5743-5745; Best, M (2009) Biochemistry 48(28): 6571-6584. In some embodiments, cysteine residues may be incorporated at various positions within the recombinant polypeptides of the disclosure to facilitate site-specific PEGylation via the cysteine side chain as described in, for example, Dozier and Distefano (2015) International Journal of Molecular Science 16(10): 25831-25864.

In certain embodiments, the present disclosure provides IL-22 variant polypeptides comprising incorporation of one or more amino acids enabling site specific PEGylation (e.g., cysteine or non-natural amino acid) of the present disclosure, wherein the amino acid substitution for site specific PEGylation site is not in the interface between the IL-22 and a component of the IL-22 receptor, e.g., IL-10Rβ or IL-22R1. In such instances, the incorporation of the site-specific amino acid modification are incorporated at IL-22 amino acid positions other than amino acid residues 41-58, 97-115, 124-131 of SEQ ID NO: 1 or SEQ ID NO: 6, which encompass the IL-10Rβ binding site revealed in the crystal structure described herein. As discussed supra, for the purpose of the present disclosure, all amino acid numbering is based on the precursor polypeptide (or pre-protein) sequence of the IL-22 protein set forth in SEQ ID NO: 1 (human IL-22) or SEQ ID NO: 6 (mouse IL-22). In some embodiments, the present disclosure provides IL-22 variant polypeptides comprising site-specific amino acid substitutions to enable site specific conjugation (e.g., PEGylation) at one or more following amino acid positions 41-58, 97-115, 124-131 of SEQ ID NO: 1 or SEQ ID NO: 6.

In some embodiments, site specific PEGylation can be employed by conjugation of PEGs to the surface of the IL-22 variant that interacts with an IL-22 binding protein (e.g., IL-10Rβ or IL22R1) so as to modulate or eliminate neutralization of the IL-22 by the IL-22 binding protein (e.g., IL-10Rβ or IL22R1). Examples of such amino acid residues within IL-22 which are involved in the interaction between IL-22 and IL-22 binding protein include, but are not limited, to amino acid residues 50-73, 166-176 of SEQ ID NO: 1 or SEQ ID NO: 6.

Incorporation of Site-Specific PEGylation Sites

In some embodiments, the interaction of the IL-22 protein with the IL-10Rβ protein may be modulated by incorporation of site specific pegylation at the amino acid locations described herein at the IL-22 interface. The incorporation of non-natural amino acids (or cysteine residues) that facilitate site specific PEGylation at one or more of positions corresponding to an amino acid residue selected from the group consisting of D43, S45, N46, Q49, Q116, R124, and R128 of SEQ ID NO: 1 of SEQ ID NO: 1 or SEQ ID NO: 6 (i.e., residues D10, S12, N13, Q16, Q83, R91, and R95 when numbered in accordance with the mature human IL-22 protein lacking the signal peptide, i.e., the sequence of SEQ ID NO: 34 or SEQ ID NO: 35) provide IL-22 variant polypeptides with modulated binding to the IL-10Rβ protein resulting in an variant IL-22/IL-22R1/IL-10Rβ receptor complex having partial agonist activity. In such instances where PEG molecules are incorporated at the interface, so as to not completely disrupt the binding of the IL-22 variant polypeptide with the IL-10Rβ or IL22R1 proteins thereby ablating activity, the PEG is typically a low molecular weight PEG species of from about 1 kDa, alternatively about 2 kDa, alternatively about 3 kDa, alternatively about 4 kDa, alternatively about 5 kDa, alternatively about 6 kDa, alternatively about 7 kDa, alternatively about 8 kDa, alternatively about 9 kDa, alternatively about 10 kDa, alternatively about 12 kDa, alternatively about 15 kDa, or alternatively about 20 kDa.

Formulations

The IL-22 variant polypeptides of the present disclosure are useful for the treatment and/or prevention of inflammatory diseases in a mammalian subject suffering therefrom by the administration to the subject of a therapeutically effective amount of such I IL-22 variant polypeptide, alone or in combination with one or more additional therapeutic agents. Additionally, the present disclosure provides a method of treating and/or preventing an inflammatory diseases in a mammalian subject suffering therefrom by the administration to the subject of a therapeutically effective amount of such IL-22 variant polypeptide, alone or in combination with one or more additional therapeutic agents.

In some embodiments, the compositions and methods of the present disclosure are useful in the treatment of inflammatory diseases of the gastrointestinal (GI) tract including but not limited to Crohn's Disease (CD), ulcerative colitis (UC), and other forms of inflammatory bowel disease (IBD) which are characterized by chronic inflammation of the intestinal tract. While Crohn's disease may affect areas of the intestinal tract from the mouth to the anus, ulcerative colitis is typically associated with inflammation of the large intestine and rectum.

Traditional first line treatments for such inflammatory intestinal conditions include 5-aminosalicyclic acid, optionally in combination with corticosteroids. The more recent development of therapeutic proteins such anti-TNF alpha antibodies (e.g. infliximab, adalimumab, golimumab) and integrin receptor antagonists (e.g., natalizumab and vedolizumab) and IL23 antagonists (e. g. Ustekinumab). While providing significant efficacy in the treatment of chronic inflammatory diseases, the conventional parenteral (IM, IV or SQ) administration of such polypeptide in systemic exposure with a comparatively small fraction of the agent being exposed to the therapeutic. Although these agents provide specific disruption of the inflammatory pathways, the systemic exposure to such agents resulting from their parenteral administration is associated with significant systemic side effects including immunosuppression leading to increased vulnerability to serious, potentially life-threatening, infections including tuberculosis, fungal infections, urinary tract infections, and lymphoma. Such significant systemic toxicities limit the dose of the agent to be administered resulting in suboptimal exposure of the affected tissue to the biologic therapeutic.

Although the selective nature of the IL-22 variant polypeptides of the present disclosure mitigate such systemic side effects when administered parenterally, in some instances it may be desirable to provide direct administration of the IL-22 variant polypeptides to the intestinal tract.

Formulation for Oral Administration of IL-22 Variant Polypeptides

The present disclosure further provides a method of treating and/or preventing an inflammatory disease of the gastrointestinal tract in a mammalian subject, the method comprising administering to the mammalian subject a therapeutically or prophyactially effective amount of a composition comprising an IL-22 variant polypeptide of the present disclosure, alone or in combination with one or more additional therapeutic agents. The present disclosure further provides an enteral composition comprising an IL-22 variant polypeptide of the present disclosure. In some embodiments, the enteral composition comprises a pharmaceutically acceptable formulation of a IL-22 variant polypeptide wherein such formulation resists degradation of the IL-22 variant polypeptide in the upper GI tract and disintegrates in the lower GI tract thus facilitating administration of the IL-22 variant polypeptide to the lower GI tract including the small intestine, large intestine, rectum and anus and treatment of inflammatory diseases of the lower GI tract including but not limited to ulcerative colitis. Exemplary polypeptide formulations suitable for oral administration are known in the art such as those described in Hamman, et al “Oral Delivery of Peptide Drugs” (2005). BioDrugs 19, 165-177; Blichmann, P. “Oral Delivery of Peptide Drugs for Mitigation of Crohn's Disease” (2012). All Theses. 1486 and reviewed in Anselmo, et al “Non-invasive delivery strategies for biologics) (2019) Nat Rev Drug Discov 18, 19-40.

Delivery of Engineered Prokaryotic Cells

In another embodiment, the present disclosure provides methods for administration of an IL-22 variant to the GI tract of a mammalian subject the method comprising the step of administering to the subject a pharmaceutically acceptable composition comprising a recombinantly engineered procaryotic cell, the procaryotic cell comprising a nucleic acid sequence encoding an IL-22 variant of the present disclosure operably linked to one or more expression control sequences such that the IL-22 variant is expressed in the recombinant procaryotic cell and the IL-22 variant released into the GI tract by secretion from or lysis of the recombinantly modified procaryotic cell or displayed on the surface of the procaryotic cell. Examples of recombinantly modified bacterial cells for the administration of IL-22 are known in the art Lin, et al. Lactobacillus delivery of bioactive interleukin-22. Microb Cell Fact 16, 148 (2017). In some embodiments, the engineered bacterial cell expressing the IL-22 variant may be administered orally, typically in aqueous suspension, or rectally (e.g. enema).

Procaryotic Viral Delivery to Intestinal Flora

In one embodiment, the present disclosure provides a recombinantly modified procaryotic virus (e.g., bacteriophage) comprising a nucleic acid sequence encoding an IL-22 variant polypeptide. In another embodiment, the present disclosure provides a method of treating an inflammatory disease of the intestinal tract of a mammalian subject, the method comprising administering to the subject a therapeutically effective amount of a pharmaceutically acceptable formulation of a recombinantly modified procaryotic virus (e.g., bacteriophage) comprising a nucleic acid sequence encoding an IL-22 variant polypeptide, the nucleic acid sequence operably linked to one or more expression control sequences functional in a host procaryotic cell for the virus such that upon infection of a procaryotic cell by the procaryotic virus, the IL-22 variant is expressed in the host cell. In one embodiment, the bacteriophage is a lytic phage such that the bacteriophage induces the lytic pathway of the bacterial cell following infection resulting local release of the IL-22 variant and delivery of the IL-22 variant to the intestinal mucosa.

As used herein, the terms ‘procaryotic virus,” “bacteriophage” and “phage” are used interchangeably hereinto describe any of a variety of bacterial viruses that infect and replicate within a bacterium. A wide variety of bacteriophages capable of selection a broad range of bacterial cells have been identified and characterized extensively in the scientific literature. Since bacteriophages exhibit significant selectivity in the bacterial cell susceptible to infection, the bacteriophage is typically selected in view of the target bacterium of the intestinal flora for expression. Samples of bacteriophages suitable as starting material for the generation of the recombinantly engineered bacteriophage against any of a wide variety of bacterial cells are available from the American Type Culture Collection (Manassas, Va.). The manipulation of the phage genome is conducted in accordance with standard methodologies for the manipulation of recombinant nucleic acids well known to those of skill in the art such as those describing in Molecular Cloning: A Laboratory Manual (available from Cold Spring Harbor Press) and conventional reagents available from a wide variety of laboratory supply houses.

The delivery of the IL-22 variant to the site of infection is achieved by a bacteriophage capable of infecting the a procaryotic cell of the intestinal flora of the subject, the bacteriophage modified using recombinant technology to introduce an expression cassette comprising a promoter active in the target procaryotic cell operably linked to a nucleic acid sequence encoding an IL22 variant, said expression cassette inserted into a non-essential region of a bacteriophage genome such that following transfection of the target bacterial cell by the recombinantly engineered bacteriophage, the IL22 variant polypeptide is expressed in the target bacterial cell. For example, in the case of bacteriophage targeting S. aureus cells, the expression cassette would employ a S. aureus promoter driving expression of the IL22 variant inserted into a non-essential region of the S. aureus specific bacteriophage. The identification of non-essential regions of the bacteriophage genome are readily identified by the known genomic organization and coding sequences of such phages. Prokaryotic promoter sequences active in a wide variety of bacteria are well known in the art. Promoter sequences may be obtained from excising naturally occurring sequences or through sequencing and synthetic synthesis of nucleic acid sequences corresponding to naturally occurring prokaryotic promoter sequences of the target bacterial cell. See, e.g. Estrem, et al. (1999) Bacterial promoter architecture: Subsite structure of UP elements and interactions with the carboxy-terminal domain of the RNA polymerase alpha subunit; Genes & Development 13(16): 2134-2147.

Codon Otimization

The amino acid sequences and corresponding encoding nucleic acid sequences if IL-22 variant are provided herein and nucleic acid sequences may be generated by one of skill in the art based on the degeneracy of the tgenetic code. In one embodiment of the invention, the mammalian sequence of the IL-22 variant is optimized for expression in the bacterial cell target environment through the use of codons optimized for expression. The techniques for the construction of synthetic nucleic acid sequences encoding IL-22 variant using and preferred codons optimal for bacterial cell expression may be determined by computational methods analyzing the commonality of codon usage for encoding native proteins of the bacteriophage genome and their relative abundance by techniques well known in the art. The codon usage database (http://www.kazusa.or.jp/codon) may be used for generation of codon optimized sequences in bacterial environments. Furthermore, a variety of software tools are available to convert sequences from one organism to the optimal codon usage for a different host organism such as the JCat Codon Optimization Tool (www.jcat.de), Integrated DNA technologies Codon Optimization Tool (https://www.idtdna.com/CodonOpt) or the Optimizer online codon optimization tool (http://genomes.urv.es/OPTIMIZER). Such synthetic sequences may be constructed by techniques well known in the art for the construction of synthetic nucleic acid molecules and may be obtained from a variety of commercial vendors.

Deletion of PAM Motifs of Phage Vector

In some embodiments, to facilitate expression of the IL-22 polypeptide from the recombinant vector in the intestinal flora, the recombinant procaryotic vector of the present invention may be modified to avoid or inhibit the defense mechanisms of the bacterial host cell. Bacterial hosts have developed defense mechanisms to guard against bacteriophage infection such as the Cas9 endonuclease which introduces a double strand DNA cleavage inactivating the phage. The Cas9 endonuclease surveys the genome to identify a protospacer adjacent motif (PAM) site which is essential for Cas9 to bind to the target DNA. As PAM sequences are essential for Cas9 function, elimination of Cas9 sequences from the procaryotic virus minimizes the ability of the Cas9 endonuclease endogenous to the subject's intestinal bacterial flora to neutralize the invading phage encoding the IL-22 variant.

Engineered Regulatory T Cells (Tregs) that Express IL-22 Variant Polypeptide

In some embodiments, the present disclosure further provides a recombinantly modified Treg cell, the Treg comprising an expression cassette comprising a nucleic acid sequence encoding an IL22 variant, optionally comprising a signal peptide, operably linked to expression control sequences functional in the Treg cell capable of directing the transcription and translation of the nucleic acid sequence encoding the IL22 variant, optionally in secreted form when associated with a signal peptide. The terms “regulatory T cell” or “Treg cell” as used herein refers to a type of CD4+ T cell that can suppress the responses of other T cells including but not limited to effector T cells (Teff). Treg cells are characterized by expression of CD4, the a-subunit of the IL-2 receptor (CD25), and the transcription factor forkhead box P3 (FOXP3) (Sakaguchi, Annu Rev Immunol 22, 531-62 (2004). Engineered Treg cells and the process for their preparation are well known in the art. See, e.g. McGovern, et al. Engineering Specificity and Function of Therapeutic Regulatory T Cells (2017). Front. Immunol. 8:1517; Scott, D. Genetic Engineering of T Cells for Immune Tolerance (2020) Molecular Therapy: Methods & Clinical Development 16:103 and include CAR Tregs as described in Zhang, et al (2018) Frontiers in Immunology 12(9):235.

Administration of IL-22 Via Viral Vector

In some embodiments, the IL-22 variant may be administered to the mammalian subject by contacting the subject with an expression vector comprising a nucleic acid sequence encoding the IL-22 variant. Expression vectors may be viral vectors or non-viral vectors. The term “nonviral vector” refers to an autonomously replicating, extrachromosomal circular DNA molecule, distinct from the normal genome and nonessential for cell survival under nonselective conditions capable of effecting the expression of an coding sequence in the target cell. Plasmids are examples of non-viral vectors. In order to facilitate transfection of the target cells, the target cell may be exposed directly with the non-viral vector may under conditions that facilitate uptake of the non-viral vector. Examples of conditions which facilitate uptake of foreign nucleic acid by mammalian cells are well known in the art and include but are not limited to chemical means (such as Lipofectamine®,Thermo-Fisher Scientific), high salt, magnetic fields (electroporation)

In one embodiment, a non-viral vector may be provided in a non-viral delivery system. Non-viral delivery systems are typically complexes to facilitate transduction of the target cell with a nucleic acid cargo wherein the nucleic acid is complexed with agents such as cationic lipids (DOTAP, DOTMA), surfactants, biologicals (gelatin, chitosan), metals (gold, magnetic iron) and synthetic polymers (PLG, PEI, PAMAM). Numerous embodiments of non-viral delivery systems are well known in the art including lipidic vector systems (Lee et al. (1997) Crit Rev Ther Drug Carrier Syst. 14:173-206); polymer coated liposomes (Marin et al., U.S. Pat. No. 5,213,804, issued May 25, 1993; Woodle, et al., U.S. Pat. No. 5,013,556, issued May 7, 1991); cationic liposomes (Epand et al., U.S. Pat. No. 5,283,185, issued Feb. 1, 1994; Jessee, J. A., U.S. Pat. No. 5,578,475, issued Nov. 26, 1996; Rose et al, U.S. Pat. No. 5,279,833, issued Jan. 18, 1994; Gebeyehu et al., U.S. Pat. No. 5,334,761, issued Aug. 2, 1994).

In another embodiment, the expression vector may be a viral vector. As used herein, the term viral vector is used in its conventional sense to refer to any of the obligate intracellular parasites having no protein-synthesizing or energy-generating mechanism and generally refers to any of the enveloped or non-enveloped animal viruses commonly employed to deliver exogenous transgenes to mammalian cells. A viral vector may be replication competent (e.g., substantially wild-type), conditionally replicating (recombinantly engineered to replicate under certain conditions) or replication deficient (substantially incapable of replication in the absence of a cell line capable of complementing the deleted functions of the virus). The viral vector can possess certain modifications to make it “selectively replicating,” i.e. that it replicates preferentially in certain cell types or phenotypic cell states, e.g., cancerous. Viral vector systems useful in the practice of the instant invention include, for example, naturally occurring or recombinant viral vector systems. Examples of viruses useful in the practice of the present invention include recombinantly modified enveloped or non-enveloped DNA and RNA viruses. For example, viral vectors can be derived from the genome of human or bovine adenoviruses, vaccinia virus, lentivirus, herpes virus, adeno-associated virus, lentivirus (e.g. human immunodeficiency virus) sindbis virus, and retroviruses (including but not limited to Rous sarcoma virus), and hepatitis B virus. Typically, genes of interest are inserted into such vectors to allow packaging of the gene construct, typically with accompanying viral genomic sequences, followed by infection of a sensitive host cell resulting in expression of the gene of interest.

To facilitate combination therapy with additional polypeptide therapeutic agents, the expression vector may encode one or more polypeptides in addition to the IL22 variant polypeptide. When expressing multiple polypeptides as in the practice of the present invention, each polypeptide may be operably linked to an expression control sequence (monocistronic) or multiple polypeptides may be encoded by a polycistronic construct where multiple polypeptides are expressed under the control of a single expression control sequence. In one embodiment, the expression vector encoding the targeting antigen may optionally further encode one or more immunological modulators. Examples of immunological modulators useful in the practice of the present invention include but are not limited to cytokines. The expressed cytokines can be directed for intracellular expression or expressed with a signal sequence for extracellular presentation or secretion.

The expression vector may optionally provide an expression cassette comprising a nucleic acid sequence encoding a “rescue” gene. A “rescue gene” is a nucleic acid sequence, the expression of which renders the cell susceptible to killing by external factors or causes a toxic condition in the cell such that the cell is killed. Providing a rescue gene enables selective cell killing of transduced cells. Thus the rescue gene provides an additional safety precaution when said constructs are incorporated into the cells of a mammalian subject to prevent undesirable spreading of transduced cells or the effects of replication competent vector systems. In one embodiment, the rescue gene is the thymidine kinase (TK) gene (see e.g. Woo, et al. U.S. Pat. No. 5,631,236 issued May 20, 1997 and Freeman, et al. U.S. Pat. No. 5,601,818 issued Feb. 11, 1997) in which the cells expressing the TK gene product are susceptible to selective killing by the administration of ganciclovir.

### Methods of the Disclosure

Administration of any one of the therapeutic compositions described herein, e.g., recombinant polypeptides, IL-22 polypeptide variants, nucleic acids, recombinant cells, and pharmaceutical compositions, can be used to treat patients in the treatment of relevant diseases, such as cancers and chronic infections. In some embodiments, recombinant polypeptides, IL-22 polypeptide variants, nucleic acids, recombinant cells, and pharmaceutical compositions as described herein can be incorporated into therapeutic agents for use in methods of treating an individual who has, who is suspected of having, or who may be at high risk for developing one or more autoimmune disease or conditions associated with IL-22 signaling. Exemplary autoimmune disease or conditions can include, without limitation, cancers, immune diseases, and chronic infection. In some embodiments, the individual is a patient under the care of a physician.

Accordingly, in one aspect, some embodiments of the disclosure relate to methods for modulating IL-22-mediated signaling in a subject, wherein the methods include administering to the subject a composition including one or more of: (a) a recombinant polypeptide of the disclosure; (b) a recombinant nucleic acid of the disclosure; (c) a recombinant cell of the disclosure; and (d) a pharmaceutically composition of the disclosure. In some embodiments, the methods include administering a therapeutically effective amount of the recombinant polypeptide of the disclosure.

In another aspect, some embodiments of the disclosure relate to methods for the treatment of a condition in a subject in need thereof, wherein the methods includes administering to the subject a composition including one or more of: (a) a recombinant polypeptide of the disclosure; (b) a recombinant nucleic acid of the disclosure; (c) a recombinant cell of the disclosure; and (d) a pharmaceutically composition of the disclosure. In some embodiments, the methods include administering a therapeutically effective amount of the recombinant polypeptide of the disclosure.

In some embodiments, the disclosed pharmaceutical composition is formulated to be compatible with its intended route of administration. The recombinant polypeptides of the disclosure may be given orally or by inhalation, but it is more likely that they will be administered through a parenteral route. Examples of parenteral routes of administration include, for example, intravenous, intradermal, subcutaneous, transdermal (topical), transmucosal, and rectal administration. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5). The parenteral preparation can be enclosed in ampoules, disposable syringes or multiple dose vials made of glass or plastic.

Dosage, toxicity and therapeutic efficacy of such subject recombinant polypeptides of the disclosure can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are generally suitable. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the disclosure, the therapeutically effective dose can be estimated initially from cell culture assays. A dose may be formulated in animal models to achieve a circulating plasma concentration range that includes the IC₅₀ (e.g., the concentration of the test compound which achieves a half-maximal inhibition of symptoms) as determined in cell culture. Such information can be used to more accurately determine useful doses in humans. Levels in plasma may be measured, for example, by high performance liquid chromatography.

The therapeutically effective amount of a subject recombinant polypeptide of the disclosure (e.g, an effective dosage) depends on the polypeptide selected. For instance, single dose amounts in the range of approximately 0.001 to 0.1 mg/kg of patient body weight can be administered; in some embodiments, about 0.005, 0.01, 0.05 mg/kg may be administered. In some embodiments, 600,000 IU/kg is administered (IU can be determined by a lymphocyte proliferation bioassay and is expressed in International Units (IU). The compositions can be administered one from one or more times per day to one or more times per week; including once every other day. The skilled artisan will appreciate that certain factors may influence the dosage and timing required to effectively treat a subject, including but not limited to the severity of the disease, previous treatments, the general health and/or age of the subject, and other diseases present. Moreover, treatment of a subject with a therapeutically effective amount of the subject recombinant polypeptides of the disclosure can include a single treatment or, can include a series of treatments. In some embodiments, the compositions are administered every 8 hours for five days, followed by a rest period of 2 to 14 days, e.g., 9 days, followed by an additional five days of administration every 8 hours.

Non-limiting exemplary embodiments of the disclosed methods for modulating IL-22-mediated signaling in a subject and/or for the treatment of a condition in a subject in need thereof can include one or more of the following features. In some embodiments, the administered composition confers a tissue-selective IL-22 signaling in the subject compared to a composition comprising a reference IL-22 polypeptide lacking the amino acid substitution(s). As described in greater detail in the Examples, an exemplary IL-22 polypeptide variant of the disclosure, 22-B3, elicits tissue-selective signaling responses in vivo, acting as a STAT3-biased agonist in the pancreas and colon but a neutral antagonist in the skin and liver. In particular, 22-B3 variant retains the tissue protective functions of IL-22 in vivo without inducing inflammatory mediators in the skin, liver, and colon, thereby uncoupling the major tissue protective and pro-inflammatory functions of IL-22. Accordingly, in some embodiments, the amino acid substitutions in the recombinant IL-22 polypeptide variants disclosed herein result in a tissue-selective IL-22 signaling which involves a reduction of IL-22 signaling in the skin while substantially retains IL-22 signaling in the pancreas and/or the gastrointestinal tract.

Accordingly, in some embodiments, the administered composition results in a tissue-selective IL-22 signaling which involves a reduction of IL-22 signaling in the skin while substantially retains IL-22 signaling in the pancreas and/or the gastrointestinal tract. In some embodiments, the tissue-selective IL-22 signaling includes a reduction of IL-22 signaling in the liver while substantially retains IL-22 signaling in the pancreas and/or the gastrointestinal tract.

In some embodiments, the administered composition results in a biased IL-22 signaling as determined by, for example, phosphorylation of STAT1 and STAT3, compared to a composition comprising a reference IL-22 polypeptide lacking the amino acid substitution(s). In some embodiments, the biased IL-22 signaling includes a reduction in a STAT1 phosphorylation by at least 10%, e.g., by at least 10%, at least 20%, at least 30%, at least 40%, at least 50%, at least 60%, at least 70%, at least 80%, or at least 90% when compared to a reference IL-22 polypeptide lacking the amino acid substitution(s). In some embodiments, the biased IL-22 signaling includes a reduction in a STAT3 phosphorylation by at least 10%, e.g., by at least 10%, at least 20%, at least 30%, at least 40%, at least 50%, at least 60%, at least 70%, at least 80%, or at least 90% when compared to a reference IL-22 polypeptide lacking the amino acid substitution(s).

In some embodiments, the administered composition results in a reduction in one or more STAT1-mediated pro-inflammatory functions. Non-limiting examples of STAT1-mediated pro-inflammatory function include cytokine production, chemokine production, and immune cell recruitment. In some embodiments, the STAT1-mediated pro-inflammatory function is reduced about 20% to about 100%, as determined by a gene expression assay, a phospho-flow signaling assay, and/or an enzyme-linked immunosorbent assay (ELISA). In some embodiments, the administered composition results in a reduction in one or more STAT1-mediated pro-inflammatory functions, as determined by the polypeptides' capacity to induce expression of a pro-inflammatory gene. Non-limiting examples of pro-inflammatory genes include CXCL1, CXCL2, CXCL8, CXCL9, CXCL10, IL-1β, and IL-6. In some embodiments, the STAT1-mediated pro-inflammatory function is reduced about 20% to about 100%, for example, about 20% to about 50%, about 30% to about 60%, about 40% to about 70%, about 50% to about 80%, about 40% to about 90%, bout 50% to about 100%, about 40% to about 80%, about 30% to about 70%, about 20% to about 80%, about 20% to about 70%, about 20% to about 60%, about 30% to about 80%, about 30% to about 90%, or about 30% to about 100% compared to a reference IL-20 lacking the amino acid substitution(s).

In some embodiments, the administered composition substantially retain one or more STAT3-mediated function. Examples of STAT3-mediated functions include, but are not limited to tissue protection, tissue regeneration, cell proliferation, and cell survival. In some embodiments, the administered composition substantially retains one or more STAT3-mediated function, as determined by the polypeptides' capacity to induce expression of a biomarker, such as Reg3β, Reg3γ, Muc1, Muc2, Muc10, BCL-2, Cyclin-D, Claudin-2, LCN2, or β-Defensin.

In some embodiments, the administered composition results in a reduction in a STAT1-mediated pro-inflammatory function while substantially retains its STAT3-mediated function. In some embodiments, the administered composition results in a ratio of STAT1-mediated signaling to STAT3-mediated signaling ranging from about 1:1.5 to about 1:10 such as, for example, about 1:2 to about 1:5, about 1:2 to about 1:7, about 1:3 to about 1:8, about 1:4 to about 1:10, about 1:4 to about 1:9, or about 1:4 to about 1:8. In some embodiments, the administered composition results in a ratio of STAT1-mediated signaling to STAT3-mediated signaling ranging from about 1.5 to 3.2. In some embodiments, the STAT1 signaling and/or STAT3 signaling is determined by an assay selected from the group consisting of by a gene expression assay, a phospho-flow signaling assay, and an enzyme-linked immunosorbent assay (ELISA).

In some embodiments, the administered composition confers a reduced capacity to induce expression of a pro-inflammatory gene selected from CXCL1, CXCL2, CXCL8, CXCL9, CXCL10, IL-1β, and IL-6 in the subject. In some embodiments, he administered composition substantially retains its capacity to induce expression of a gene selected from Reg3β, Reg3γ, Muc1, Muc2, Muc10, BCL-2, Cyclin-D, Claudin-2, LCN2, and β-Defensin in the subject. In some embodiments, the administration of the pharmaceutical composition does not inhibit T-cell activity in the subject.

In some embodiments, the administered composition enhances epithelial protection and regeneration.

In some embodiments, the immune disease is an autoimmune disease. In some embodiments, the autoimmune disease is selected from the group consisting of rheumatoid arthritis, insulin-dependent diabetes mellitus, hemolytic anemias, rheumatic fever, thyroiditis, Crohn's disease, myasthenia gravis, glomerulonephritis, autoimmune hepatitis, multiple sclerosis, alopecia areata, psoriasis, vitiligo, dystrophic epidermolysis bullosa, systemic lupus erythematosus, graft vs. host disease, ulcerative colitis, pancreatitis, psoriatic arthritis, and diabetic foot ulcer. In some embodiments, the autoimmune disease is acute pancreatitis. In some embodiments, the subject is a mammal. In some embodiments, the mammal is a human. In some embodiments, the subject has or is suspected of having a condition associated with IL-22 mediated signaling.

### Additional Therapies

As discussed supra, any one of the recombinant polypeptides, nucleic acids, recombinant cells, cell cultures, and/or pharmaceutical compositions described herein can be administered in combination with one or more additional therapeutic agents such as, for example, immunosuppressive agents, immunosuppressants, or anti-inflammatory agents. Administration “in combination with” one or more additional therapeutic agents includes simultaneous (concurrent) and consecutive administration in any order. In some embodiments, the one or more additional therapeutic agents, immunosuppressants, or anti-inflammatory agents is selected from the group consisting of chemotherapy, radiotherapy, immunotherapy, hormonal therapy, toxin therapy, surgery, and disease modifying anti-rheumatic drugs (DMARDs). Various classes of anti-inflammatory agents can be used. Non-limiting examples include cytokine antagonists, cytokine receptor antagonists, soluble receptors, integrin antagonists, corticosteroids, kinase inhibitors, S1P agonists, and PDE4 antagonists.

In some embodiments, the methods of treatment as described herein further include administration of a composition that suppresses inflammatory immune responses, e.g., anti-inflammatory drugs. Suitable anti-inflammatory drugs include, but are not limited to, TNF antagonists (e.g., adalimumab, infliximab), IL-23 antagonists (e.g., ustekinumab), IL-17 antagonists (e.g. secukinumab), IL-1 antagonists (e.g. anakinra), IL-6 antagonists (e.g. tocilizumab), IL-12 antagonists (e.g., ustekinumab), IL-2 antagonists (e.g., Daclizumab), integrin antagonists (e.g., vedolizumab), corticosteroids (e.g. prednisone), aminosalicylates (e.g. mesalamine, balsaazide, olsalazine), methotrexate, azathioprine, leflunomide, chloroquine, calcineurin inhibitors (cyclosporine, tacrolimus), abatacept, rituximab, JAK inhibitors (e.g., tofacitinib), PDE4 inhibitors (e.g., apremilast), mTOR inhibitors (e.g., sirolimus), S1P receptor agonists (e.g., Ozanimod).

In some embodiments, the methods of treatment as described herein further include administration of a composition comprising a tissue regenerative factor such as a Wnt agonist or a Notch agonist.

Accordingly, in some embodiments of the methods disclosed herein, the composition is administered to the subject individually as a first therapy or in combination with a second therapy. In some embodiments, the second therapy is selected from the group consisting of DMARDs, immunotherapy, hormonal therapy. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

### Kits

Also provided herein are kits including the IL-22 partial agonists, recombinant nucleic acids, recombinant cells, or pharmaceutical compositions provided and described herein as well as written instructions for making and using the same. For example, provided herein, in some embodiments, are kits that include one or more of: a recombinant polypeptide of the disclosure, an IL-22 partial agonist of the disclosure, a recombinant nucleic acids of the disclosure, a recombinant cell of the disclosure, or a pharmaceutical composition of the disclosure; and instructions for use thereof. In some embodiments, the kits of the disclosure further include one or more syringes (including pre-filled syringes) and/or catheters (including pre-filled syringes) used to administer one any of the provided recombinant nucleic acids, recombinant cells, or pharmaceutical compositions to an individual; and instructions for use thereof . In some embodiments, a kit can have one or more additional therapeutic agents that can be administered simultaneously or sequentially with the other kit components for a desired purpose, e.g., for modulating an activity of a cell, inhibiting a target cancer cell, or treating a disease in an individual in need thereof

Any of the above-described kits can further include one or more additional reagents, where such additional reagents can be selected from: dilution buffers; reconstitution solutions, wash buffers, control reagents, control expression vectors, negative control polypeptides, positive control polypeptides, reagents for in vitro production of the recombinant polypeptides. In some embodiments, the components of a kit can be in separate containers. In some other embodiments, the components of a kit can be combined in a single container. For example, in some embodiments of the disclosure, the kit includes one or more of the recombinant I IL-22 polypeptides, recombinant nucleic acids, recombinant cells, or pharmaceutical compositions as described herein in one container (e.g., in a sterile glass or plastic vial) and a further therapeutic agent in another container (e.g., in a sterile glass or plastic vial).

In some embodiments, a kit can further include instructions for using the components of the kit to practice a method described herein. For example, the kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the disclosure may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and intellectual property information.

In some embodiments, a kit can further include instructions for using the components of the kit to practice the methods. The instructions for practicing the methods are generally recorded on a suitable recording medium. For example, the instructions can be printed on a substrate, such as paper or plastic, etc. The instructions can be present in the kit as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

### EXAMPLES

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, N.Y.: Cold Spring Harbor Laboratory and Sambrook, J., & Russel, D. W. (2001). Molecular Cloning: A Laboratory Manual (3rd ed.). Cold Spring Harbor, N.Y.: Cold Spring Harbor Laboratory (jointly referred to herein as “Sambrook”); Ausubel, F. M. (1987). Current Protocols in Molecular Biology. New York, N.Y.: Wiley (including supplements through 2014); Bollag, D. M. et al. (1996). Protein Methods. New York, N.Y.: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, Calif.: Academic Press; Lefkovits, I. (1997). The Immunology Methods Manual: The Comprehensive Sourcebook of Techniques. San Diego, Calif.: Academic Press; Doyle, A. et al. (1998). Cell and Tissue Culture: Laboratory Procedures in Biotechnology. New York, N.Y.: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, N.Y.: Cold Spring Harbor Laboratory Press; Beaucage, S. L. et al. (2000). Current Protocols in Nucleic Acid Chemistry. New York, N.Y.: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

Additional embodiments are disclosed in further detail in the following examples, which are provided by way of illustration and are not in any way intended to limit the scope of this disclosure or the claims.

### Example 1

### General Experimental Procedures

### Protein Production and Purification

For yeast-binding studies and affinity maturation, the ECDs of mouse IL22R1 (16-228) and IL10Rβ (20-220) were cloned into the pAcGP67a baculoviral vector with an N terminal GP64 signal peptide, C-terminal 3C cleavage site followed by a biotin-acceptor peptide tag (BAP tag, GLNDIFEAQKIEW, SEQ ID NO: 33) and 6× His tag. The baculovirus stocks were prepared by cotransfection of the BaculoSapphire DNA (Orbigen) and the pAcGP67-A DNA into Spodoptera frugiperda (Sf9). Next, the viruses were used to infect the Trichoplusia ni (Hi5) cells. The proteins were purified from the supernatant of baculovirus infected Hi5 cells 48 hours after infection and purified with Ni-NTA resin (Qiagen) followed by size-exclusion chromatography (SEC) on a Superdex 200 column (GE). The proteins were maintained in HEPES buffered saline (HBS, 20 mM HEPES pH 7.4, 150 mM sodium chloride). IL10Rβ ECD was site-specifically biotinylated at the C-terminal BAP tag using BirA ligase and re-purified by size exclusion chromatography.

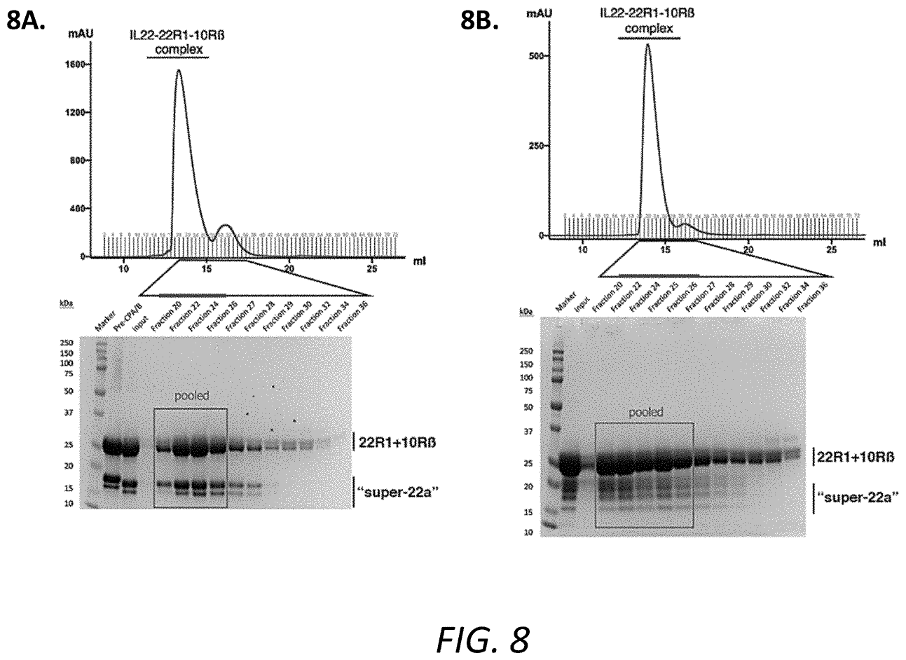

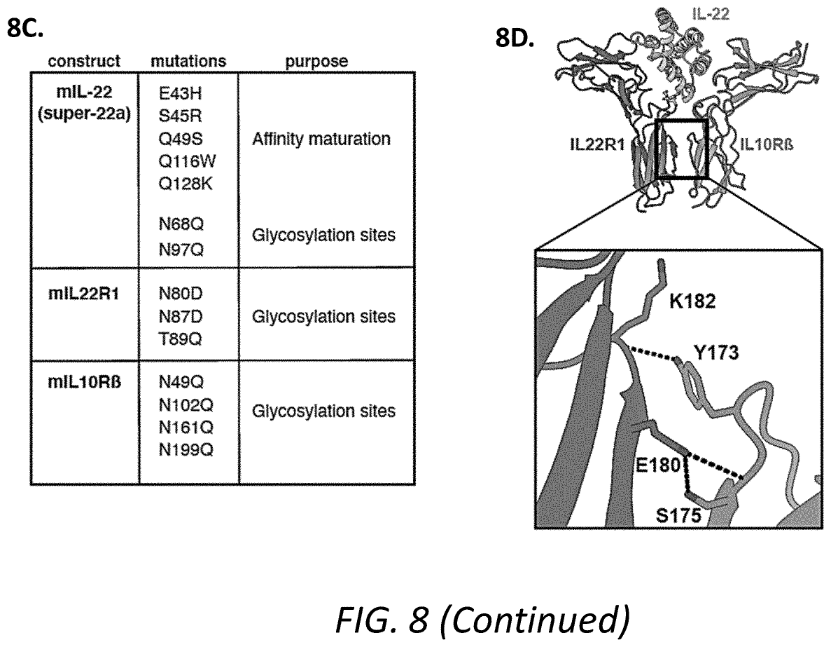

For crystallographic studies, the fully glycosylated Super-22b and glycomutant Super-22a versions of affinity matured IL-22, as well as glycomutant versions the IL22R1 and IL10Rβ ECDs (FIG. 8A were cloned into the pAcGP67a baculoviral vector with an N terminal GP64 signal peptide and C-terminal 6× His tag, expressed and purified as described above, followed by size-exclusion chromatography (SEC) on a Superdex 75 column (GE). Following SEC, the individual proteins were incubated overnight at a 1:1:1.2 molar ratio of IL-22:IL22R1:IL10Rβ in the presence of carboxypeptidase A and B before purification by SEC on an S200 column (GE).

For signaling experiments, mouse IL-22 variants were cloned into the pD649 mammalian expression vector containing an N-terminal HA signal peptide and C-terminal 6× His-tag. DNA was transiently transfected into Expi-293F cells (Thermo Fischer Scientific) using Expifectamine transfection reagent (Thermo). 96 hours after transfection, cell supernatant was harvested and proteins were purified with Ni-NTA resin (Qiagen) followed by size-exclusion chromatography (SEC) on a Superdex 75 column (GE) in HEPES buffered saline (HBS, 30 mM HEPES pH 7.4, 150 mM sodium chloride). For in vivo studies, endotoxin was removed using the NoEndoHC Column Kit (Proteus), and endotoxin removal was confirmed using the Pierce™ Chromogenic Endotoxin Quant Kit (Thermo Fisher Scientific).

### Yeast Display, Library Assembly, and Affinity Maturations

Mouse IL-22 (residues 34-179) containing a C-terminal Myc-tag was displayed on the surface of S. cerevisiae strain EBY100 as a C-terminal fusion to Aga2 using the pCT302 vector. A mutant mIL-22 library containing six randomized residues at the predicted IL10Rβ contact site was generated by primer assembly PCR using degenerate codons (NNK). Electroporation, rescue and expansion of the yeast library were performed as described previously (Chao et al., 2006). The final library contained approximately 6.5×10⁸ yeast transformants.

Library selection was conducted as described previously with some modifications. Briefly, the initial selections (rounds 1-4) were conducted using magnetic activated cell sorting (MACS). For rounds 1 and 2, 5×10⁹ and 1×10⁸ cells, respectively, were pre-incubated in 1 μM mIL22R1 and selected with paramagnetic streptavidin microbeads (Miltenyi) that were pre-coated with 400 nM biotinylated IL10Rβ. IL10Rβ-binding clones were isolated using MACS LS columns (Miltenyi). For round 3, 1.0×10⁸ yeast were pre-incubated in 1 μM mIL22R1 and stained and selected with 500 nM monomeric biotinylated IL10Rβ labeled with streptavidin conjugated to Alexa Fluor 647. IL10Rβ-binding clones were isolated using MACS LS columns (Miltenyi) in combination with Anti-Cy5/Anti-Alexa Fluor 647 MicroBeads (Miltenyi). In rounds 4 and 5, library selection was performed using two-color fluorescence activated cell sorting (FACS) to normalize apparent affinity by protein expression on the cell surface. The yeast library was pre-incubated with 1 μM mIL22R1 followed by 100 nM and 10 nM of biotinylated mIL10Rβ, for round 4 and 5, respectively. The cells were washed twice with PBE (PBS, pH 7.4%+0.5% (w/v) BSA+2 mM EDTA, pH 8.0) and co-labeled with streptavidin-Alexa Fluor 647 (mIL10Rβ binding) and anti-c-Myc-Alexa Fluor 488 (mIL-22 variant cell surface expression) for 15 min at 4° C. Alexa647⁺Alexa488⁺ yeast were purified using a SH800S Cell Sorter (Sony Biotechnology). At the conclusion of the selections, post-selection library at each round was simultaneously expanded and incubated with varying concentrations of biotinylated mIL10Rβ for 1 hour at 4° C., washed twice with PBE, then stained with fluorescently labeled streptavidin for 10 min at 4° C. to assess the enrichment of high-affinity clones via flow cytometry. In addition, 100 μL of post-round 5 library was used to extract library DNA using the Zymoprep Yeast Plasmid Miniprep II Kit (Zymo Research), according to the manufacturer's instructions. The extracted DNA was transformed into DHSα E. coli and plated to sequence the individual clones.

To measure relative binding affinities, individual clones were displayed on the yeast surface and incubated with 1 μM mIL22R1 and increasing concentrations of biotinylated mIL10Rβ. The cells were then stained with streptavidin-Alexa Fluor 647 for 15 minutes and analyzed on a CytoFLEX Flow Cytometer (Beckman Coulter).

### Crystallization, Data Collection, and Refinement

Following purification, the IL-22/IL22R1/IL10Rβ complex was concentrated to 10 mg/ml. Crystals of the fully glycosylated “Super-22b” complex were grown by hanging drop vapor diffusion with 1 μl of protein mixed with an equal volume of reservoir solution containing 0.2 M sodium potassium tartrate, 15% PEG 3350, and 0.1 M HEPES pH 7.8 at 20° C. These crystals were subsequently microseeded into hanging drops containing 1 μl of the glycomutant “Super-22a” complex, mixed with an equal volume of 0.2 M sodium potassium tartrate, 12.5% PEG 3350, and 0.1 M HEPES pH 8.0 at 20° C. Crystals were harvested and cryoprotected in mother liquor containing 25% glycerol.

Diffraction data was collected at the Advanced Photon Source (APS) beamline 23 ID-B. The 2.6 Å dataset was integrated and scaled using XDS (Kabsch, 2010) before merging symmetry-related reflections with aimless (Evans, 2011; Winn et al., 2011). Phases were solved by molecular replacement with Phaser (Adams et al., 2010; McCoy et al., 2007) using the crystal structures of human IL-22, IL-22R1 (PDB ID: 3DGC) and IL10Rβ (PDB ID: 3LQM) as search models. Model building was carried out using iterative rounds of reciprocal space refinement in Phenix. refine (Adams et al., 2010; Terwilliger et al., 2008) and manual model building in Coot (Emsley et al., 2010). All data-processing steps were carried out with programs provided through SBgrid (Morin et al., 2013). Statistics for data collection and refinement are provided in Table 3 below. Protein-protein and protein-ligand interfaces were analyzed using PDBePISA (Krissinel and Henrick, 2007). All structure figures were made using ChimeraX (Goddard et al., 2018).

TABLE 3
Data collection and refinement statistics.

| Protein | IL-22/IL-22R1/IL-10Rβ complex | |
|---|---|---|
| Organism | Mus musculus | |
| PDB ID | 6WEO | |
| Data collection | | |
| Space group | P1 | |
| a, b, c (Å) | 134.49, 145.22, 152.04 | |
| α, β, γ (°) | 71.06, 81.84, 62.48 | |
| Wavelength (Å) | 1.03317 | |
| Resolution range (Å) | 48.09-2.60 | (2.64-2.60) |
| Total reflections | 831,807 | |
| Unique reflections | 289,478 | |
| Completeness (%) | 97.5 | (97.6) |
| Redundancy | 2.9 | (3.0) |
| Anomalous completeness (%) | — | |
| R_{merge} (%) | 9.5 | (>100) |
| Rₘₑₐₛ (%) | 11.6 | (>100) |
| R_{p.i.m.} (%) | 6.6 | (93.3) |
| I/σ | 6.5 | (0.8) |
| CC_{1/2} (%) | 99.4 | (33.1) |
| Refinement | | |
| Resolution range (Å) | 48.09-2.60 | (2.67-2.60) |
| R_{work} (%) | 23.6 | |
| R_{free} (%) | 29.6 | |
| Number of Reflections: | | |
| Total | 289,145 | |
| R_{free} reflections | 1,998 | |
| Number of non-hydrogen atoms | 53,152 | |
| Protein atoms | 53,095 | |
| Water atoms | 57 | |
| R.m.s. deviations: | | |
| Bond lengths (Å) | 0.011 | |
| Bond angles (°) | 1.323 | |
| Average B factors (Å²): | | |
| Protein | 101.13 | |
| Water | 74.11 | |
| Wilson B-factor (Å²) | 69.1 | |
| Ramachandran (%): | | |

| Favored (%) | 95.36 |
|---|---|
| Allowed (%) | 3.98 |
| Outlier (%) | 0.66 |
| Clashscore | 14.14 |
| Molprobity score | 2.76 |
| Molprobity percentile | 65^{th} |

### Cell Culture

HEK-293T (ATCC CRL-3216), HT-29 (ATCC HTB-38), Panc-1 (ATCC CRL-1469), HepG2 (ATCC HB-8065), and EC4 (gift from D. Felsher) cells were all grown in DMEM (Dulbecco's Modified Eagle Medium) supplemented with 10% v/v fetal bovine serum, penicillin-streptomycin, 1 mM sodium pyruvate, 10 nM HEPES and 2 mM GlutaMAX™. The cells were maintained at 37° C. with 5% CO2. Expi293F cells were grown in serum free Expi293 expression media (Thermo) and maintained at 37° C. with 5% CO2.

### Phospho-Flow Signaling Assays

HT-29 cells were plated in 96-well plates and stimulated with WT or mutant IL-22 for 20 min at 37° C., followed by fixation with paraformaldehyde (Electron Microscopy Sciences) for 10 min at room temperature. The cells were permeabilized for intracellular staining by treatment with ice cold methanol (Fisher) for 30 min at −20° C. The cells were then incubated with the desired antibodies at a 1:50 dilution for 1 hour at room temperature in autoMACS buffer (Miltenyi). The background fluorescence of the unstimulated samples was subtracted from all samples. Data was acquired using CytoFlex, flow cytometer instrument (Beckman Coulter). The MFI values were normalized to the maximal WT IL-22 value within each experiment and plotted in Prism 7 (GraphPad). The dose-response curves were generated using the “sigmoidal dose-response” analysis. Alexa Fluor 488 Mouse Anti-Stat1 (pY701) and Alexa Fluor 647 Mouse Anti-Stat3 (pY705) antibodies were purchased from BD Biosciences.

### Immunoprecipitation and Western Blot

For signaling and immunoprecipitation experiments in HEK-293T cells, 0.6×10⁶ cells were plated in 6-well culture dishes coated with fibronectin (Millipore). 24 hours later, cells were transfected using FuGene 6 (Promega) with pLV-EF1a-IRES-Puro vector containing HA-tagged IL22R1, WT or YtoF mutant.

48 hours after transfection, cells were treated with IL-22 (WT or mutant) for 20 minutes at 37° C. Cells were then rinsed one time with ice-cold PBS and immediately lysed with Triton lysis buffer (1% Triton, 20 mM HEPES pH 7.4, 150 mM NaCl, 1 tablet of PhosSTOP phosphatase inhibitor cocktail (Roche), and 1 tablet of EDTA-free protease inhibitor (Roche) (per 10 ml buffer). The cell lysates were cleared by centrifugation at 13,000 rpm at 4° C. in a microcentrifuge for 10 minutes. For anti-HA-immunoprecipitations, the magnetic anti-HA beads (Pierce) were washed 3 times with lysis buffer. Twenty microliters of anti-HA beads was then added to clarified cell lysates and incubated with rotation for 2 hour at 4° C. Following immunoprecipitation, the beads were washed 4 times with lysis buffer. Immunoprecipitated proteins and cell lysates were denatured by the addition of SDS sample buffer and boiling for 5 minutes, resolved by SDS-PAGE, and analyzed by immunoblotting.

For western blot based signaling assays in HT-29 (1×10⁶ cells/well), HepG2 (0.6×10⁶ cells/well), and Panc-1 (0.6×10⁶ cells/well) cells were seeded in 6-well culture dishes. 24 hours later cells were treated with IL-22 (WT or mutant) for 20 minutes at 37° C., and lysates were prepared and analyzed as described above.

### In-Vivo Signaling, RT-PCR, and Serum Analysis

For in vivo signaling experiments, C5BL/6J mice were administered 200 uL of PBS or IL-22 variants (200 ug/mouse) via I.P. injection. After 30 minutes, mice were euthanized and the pancreas, colon, liver, and tail skin tissues were isolated and flash frozen in liquid nitrogen. Tissues were disrupted with mortar and pestle in liquid nitrogen and 50 mg tissue was lysed in ice-cold RIPA lysis buffer (Thermo) supplemented with 1 tablet PhosSTOP (Roche) and one tablet of protease inhibitor cocktail (Roche) per 10 mL. Lysates were further homogenized via centrifugation through a QiaShredder column (Qiagen). Protein concentrations for each sample of a given tissue were normalized by Bradford assay (Bio-Rad) before being denatured in SDS sample buffer and resolved by SDS-PAGE and analyzed by immunoblot.

For gene expression analysis in mouse tissues, C5BL/6J mice were administered 200 uL of PBS or IL-22 variants (200 ug/mouse) via I.P. injection. After 6 hours, mice were euthanized, and tissues were processed and homogenized as described above. RNA from each tissue was isolated using RNAeasy plus mini kit (Qiagen) per the manufacturer's instructions. 1 μg RNA for each sample was used for cDNA generation with iScript Reverse Transcription Supermix (BioRad). Relative gene expression was measured by SYBR-green based quantitative PCR (qPCR) using the comparative Ct method and normalized to GAPDH expression. All samples were run in triplicate. The following mouse qPCR primers were designed and ordered from Integrated DNA Technologies (IDT):

GAPDH:
(SEQ ID NO: 15)
5′GTG GAG TCA TAC TGG AAC ATG TAG3′,
and
(SEQ ID NO: 16)
5′AAT GGT GAA GGT CGG TGT G3′.
IL22R1:
(SEQ ID NO: 17)
5′CTC GTA TTC TCT CTG TTT GCC T3′,
and
(SEQ ID NO: 18)
5′CAT GAC CTG TTC TAC CGC TTA G3′.
IL10Rβ:
(SEQ ID NO: 19)
5′CAG GAC GGA GAC TAT GAG GAT3′,
and
(SEQ ID NO: 20)
5′AC A GAA CAG GAG AGT GGA GT3′.
Reg3β:
(SEQ ID NO: 21)
5′TGT TAC TCC ATT CCC ATC CAC3′,
and
(SEQ ID NO: 22)
5′CTG AGG CTT CAT TCT TGT CCT3′.
Reg3g:
(SEQ ID NO: 23)
5′GAT TCG TCT CCC AGT TGA TGT3′,
and
(SEQ ID NO: 24)
5′CTC CAT GAC CCG AC A CTG3′.
MUC1:
(SEQ ID NO: 25)
5′GAC TGC TAC TGC CAT TAC CTG3′,
and
(SEQ ID NO: 26)
5′CCT ACC ATC CTA TGA GTG AAT ACC3′.
CXCL1:
(SEQ ID NO: 27)
5′GTG CCA TCA GAG CAG TCT3′,
and
(SEQ ID NO: 28)
5′CCA AAC CGA AGT CAT AGC CA3′.
IL-6:
(SEQ ID NO: 29)
5′TCC TTA GCC ACT CCT TCT GT3′,
and
(SEQ ID NO: 30)
5′AGC CAG AGT CCT TCA GAG A3′.
IL-1β:
(SEQ ID NO: 31)
5′CTC TTG TTG ATG TGC TG3′,
(SEQ ID NO: 32)
5′GAC CTG TTC TTT GAA GTT GAC G3′.

For serum analysis of acute phase response proteins, C5BL/6J mice were administered 200 uL of PBS or IL-22 variants (2 μg or 200 μg per mouse) via I.P. injection. After 24 hours, blood was obtained by cardiac puncture, and serum was isolated by centrifugation at 2000 RPM for 10 minutes at 4° C. Levels of SAA-1/2 (Invitrogen) and Haptoglobin (R&D) were measured by ELISA following the manufacturer's instructions. Each sample was run in triplicate.

### Acute Pancreatitis Model

Acute Pancreatitis was induced in C57BL/6 mice by injection of 100 μl caerulein (50 μg/kg) once per hour for six hours. Control mice were injected with 100 μl PBS at the same time points. Mice were pretreated with PBS or 50 μg/mouse of IL-22 (WT or 22-B3) via I.P. injection 20 hours and 2 hours before the first caerulein injection. One hour after the last caerulein injection, blood was drawn by submandibular puncture, and serum was isolated by centrifugation at 2000 RPM for 10 minutes at 4° C. Serum levels of Pancreatic Lipase and Amylase were analyzed by the Stanford Veterinary Service center.

### Example 2

### Engineering a High-Affinity IL-22 Via Directed Evolution

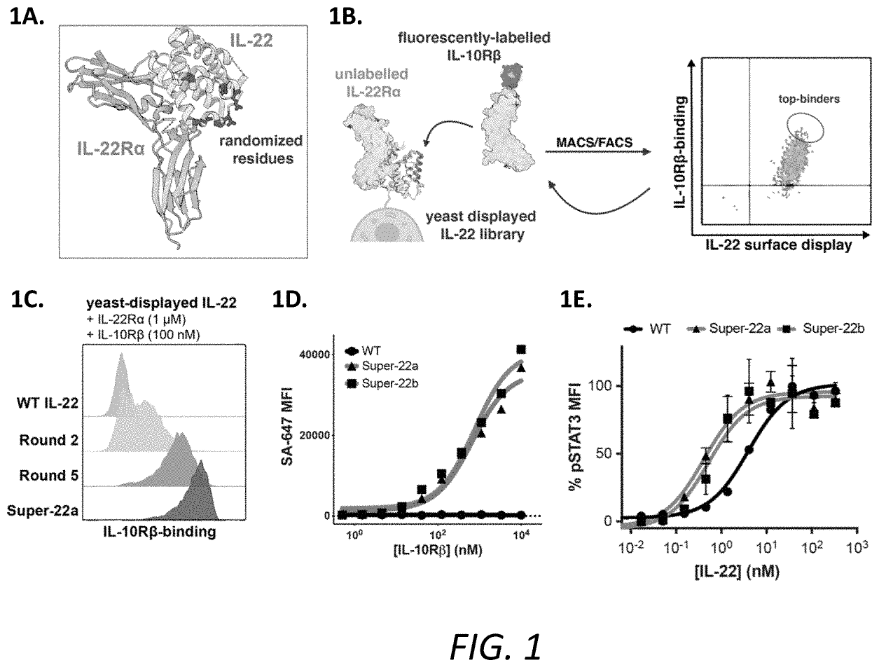

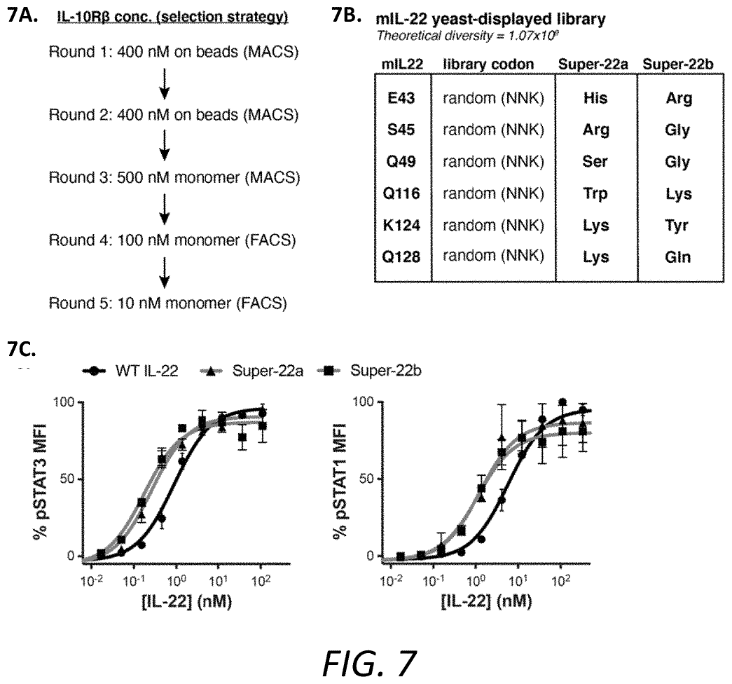

This Example describes the results of experiments performed to engineer an exemplary high-affinity IL-22 in accordance with some non-limiting embodiments of the disclosure via directed evolution approach. In order to stabilize the assembly of the ternary IL-22 receptor complex for structural studies, a yeast surface display to affinity mature the IL-22/IL10Rβ interaction was used (Angelini et al., 2015). A site-directed library was designed in which six amino acid positions in mouse IL-22 were selected for randomization. These amino acids were predicted to be in close proximity to the IL10Rβ binding interface based on information from the partial complex structure of IL-22 bound to IL22R1 as well as homology to interferon-gamma (IFN)-λ (see, e.g., FIG. 1A. This mutant IL-22 library was displayed on the surface of yeast, pre-bound with the unlabeled extracellular domain (ECD) of IL22R1, and selected with the ECD of IL10Rβ (see, e.g., FIG. 1B). This process was repeated over five rounds of in vitro evolution, yielding progressive improvements in IL10Rβ binding (see, e.g., FIG. 1C and FIG. 7A).

Two high-affinity clones were selected from the final yeast population, each containing five mutations relative to wild-type mouse IL-22, named Super-22a and Super-22b (see, e.g., FIG. 7B). Both clones exhibited significantly improved binding to the IL10Rβ ECD when displayed on the surface of yeast (see, e.g., FIG. 1D). As illustrated in FIG. 1D, affinity matured IL-22 variants demonstrated enhanced binding to IL10Rβ. Binding titration of SA-647-IL10Rβ ECD on yeast displaying WT IL-22 or affinity matured clones, Super-22a and Super-22b are shown. In these experiments, yeast were pre-bound with 1 μM unlabeled mIL22R1 ECD.

In addition, both clones displayed enhanced activity in cultured cells, with approximately 5 to 10-fold reduced EC₅₀ on both mouse and human epithelial-derived cell lines, as determined by phosphorylation of STAT1 and STAT3 (see, e.g., FIGS. 1E and 7C). As illustrated in FIG. 1E, affinity matured IL-22 variants could elicit enhanced STAT3 signaling. In these experiments, dose response curve for phospho-Y705-STAT3 in EC4 (mouse, liver) cells stimulated with WT IL-22 or indicated variants for 20 minutes and analyzed by flow cytometry following fixation and permeabilization with paraformaldehyde/methanol. Thus, these engineered high affinity IL-22 variants showed substantially improved binding to IL10Rβ while retaining the ability to functionally dimerize both receptor subunits.

### Example 3

### Crystal Structure of the Heteromeric IL-22/IL22R1/IL10Rβ Complex

This Example describes the results of experiments performed to determine the crystal structure of the heteromeric receptor complex IL-22/IL22R1/IL10Rβ, which in turns helps elucidate the chemistry that drives each of the cytokine-receptor interactions of the heteromeric receptor complex.

In contrast to wild-type IL-22, both high affinity IL-22 clones described in Example 2 above (Super-22a and Super-22b) formed a stable ternary complex with IL22R1 and IL10Rβ, as assessed by co-migration during gel filtration (FIGS. 8A and 8B). It was observed that a complex containing fully glycosylated Super-22b bound to glycomutant variants of IL22R1 and IL10Rβ yielded poorly diffracting crystals, which were ultimately used as a seed stock to facilitate the crystallization of the complex containing a glycomutant variant of Super-22a (see, e.g., FIG. 7C). These crystals diffracted to 2.6 Å resolution, and the structure was determined by molecular replacement using models derived from previously reported structures of the partial IL-22-IL22R1 complex (PDB ID: 3DGC, Jones et al., 2008) and monomeric IL10Rβ (PDB ID: 3LQM, Yoon et al., 2010).

It was observed that the overall architecture of the IL-22/IL22R1/IL10Rβ ternary complex is similar to that of other class 2 cytokine receptor complexes and consists of three distinct binding interfaces (sites 1-3, FIGS. 2A-2C). At site 1, multiple loops from both Class 2 Homology Region (C2HR) domains of IL22R1 (D1 and D2) engage IL-22 helices A and E, as well as loop L2, burying 961 Å² of the IL-22 surface. The lower affinity site 2 contact is formed by both D1 and D2 of IL10Rβ, which engage IL-22 primarily through loops L2, L3 and L4 in D1, along with L5 and L6 in D2. L5 of IL10Rβ forms extensive contacts with the bottom of IL-22 Helix A, while Loops L2, L3 and L4 engage Helix C (FIGS. 2A and 2B). Finally, site 3 consists of the receptor-receptor “stem contacts” between the D2 domains of IL22R1 and IL10Rβ, forming a smaller interface with only three hydrogen bond contacts and 421 Å² of buried surface area (see, e.g., FIG. 8D).

Both IL22R1 and IL10Rβ are shared receptor subunits that bind to additional cytokines in the IL-10 superfamily in the context of other heterodimeric receptor complexes. Previously, IL22R1 has been crystallized in complex with IL-24 and IL20Rβ, and IL10Rβ has been crystallized with IFN-λ and IFNλR1. The IL-22 Receptor structure presented herein reveals several notable differences with these related complexes. In particular, the site 2 contact formed between IL10Rβ and IL-22 differs substantially from that observed in the IFN-λ complex. In the IFN-λ bound structure, IL10Rβ engages primarily the N-terminal edge of helix C in IFN-λ, appearing to clasp the “front” of the cytokine, whereas IL10Rβ engages IL-22 via the central portion of Helix C. This differential binding mode can be readily observed when the two complexes are aligned via superposition of the cytokine ligand, showing an approximately 40° relative rotation of the IL10Rβ D1 domains between the complexes (FIG. 2D). The altered orientation of IL10Rβ in these two complexes also results in a change in the relative positioning of three aromatic residues in IL10Rβ, Tyr59, Tyr82, and Trp143, which form important contacts with the ligand in both structures (FIG. 2E).

In these experiments, despite the relatively low affinity of IL-22 for IL10Rβ, it was observed that this unique site 2 contact forms an extensive interface burying 723 Å² of the IL-22 surface. A close-up view of this interface reveals several key residues in IL-22 that mediate the interaction with IL10Rβ (FIGS. 3A and 3B).

In particular, on Helix A of IL-22, 22-Arg55 forms a salt-bridge with Rβ-Glu141, which simultaneously contacts the adjacent N-linked glycan modification on 22-Asn54 (FIG. 3A). In addition, 22-Tyr51 on Helix A makes multiple contacts with IL10Rβ, including hydrogen bonds with both Rβ-Lys81 and Rβ-Glu139, as well as a perpendicular pi-stacking interaction with Rβ-Trp143. Moreover, 22-Glu117 and 22-Lys124 in Helix C form ionic contacts with Rβ-Lys81, Rβ-Asp84, and Rβ-Glu109 in loops L3 and L4 of IL10Rβ (FIGS. 3A and 3B). Near the C-terminal edge of Helix C, 22-Gln48 in Loop L1 forms an additional hydrogen bond with Rβ-Asp150 in the D2 domain.

In addition to the contacts described above, without being bound to any particular theory, the crystal structure described herein reveals the basis for the affinity enhancement resulting from the yeast-display selected mutations (see, e.g., FIG. 7B). In particular, it was observed that the S45R mutation in Super-22a converts what is likely a hydrogen bond contact with Rβ-Gln198 in WT IL-22, into a salt-bridge with Rβ-Asp197 observed in the structure presented in FIG. 3B. In addition, the Q96S mutation forms a hydrogen bond contact with Rβ-Tyr82, while the Q116W mutation is primarily forming a hydrogen-bond contact via the indole nitrogen with the side chain of Rβ-Ser80 (FIG. 3A). In particular, both the E43H and Q128K appear to only be contacting other residues within IL-22, potentially stabilizing the orientation of loop L1 at the 10Rβ interface (FIG. 3B). Overall, without being bound to any particular theory, it appears that the affinity enhancing mutations in Super-22a are primarily improving upon pre-existing polar contacts made between WT IL-22 and IL10Rβ, rather than creating novel contact sites.

### Example 4

### Structure-Guided Design of Biased IL-22 Receptor Agonists

This Example describes experiments performed to design biased IL-22 receptor agonists based on the crystal structure of the heteromeric IL-22/L22R1/IL10Rβ complex described in Example 3 above.

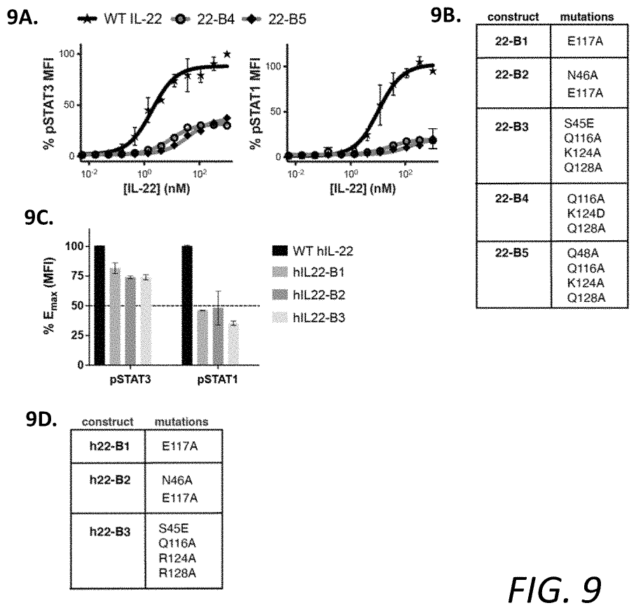

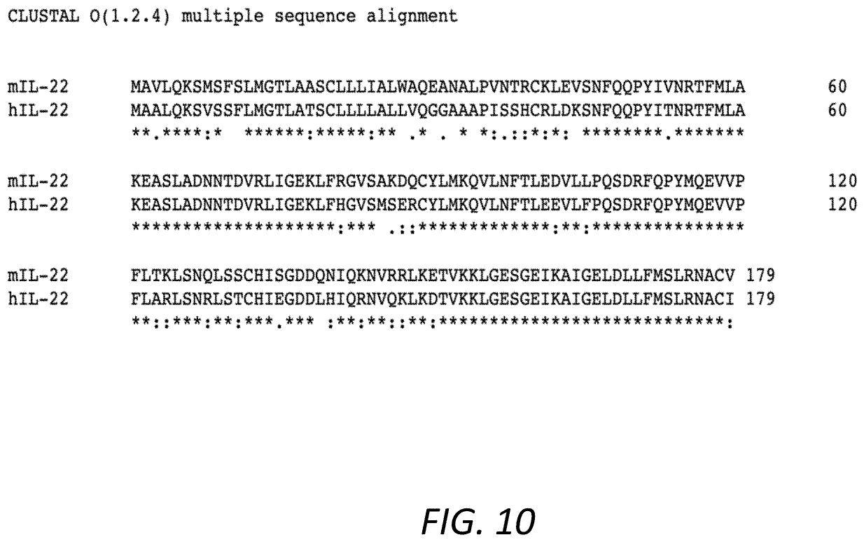

In these experiments, a series of IL-22 variants with amino acid substitutions targeting the IL10Rβ-binding site was designed and assessed for their ability to induce activation of STAT3 and STAT1 in colonic epithelial derived HT-29 cells. As illustrated in FIG. 3D, IL-22 variants that partially disrupted ILiORB-binding retained the capacity to elicit activation of STAT3 but induce substantially reduced activation of STAT1. In these experiments, dose response curves for phospho-Y705-STAT3 (left panel) and phospho-Y701-STAT1 (right panel) in HT-29 cells stimulated with WT IL-22 or indicated variants for 20 minutes and analyzed by flow cytometry following fixation and permeabilization with paraformaldehyde/methanol. Data are mean+/−SD for three independent replicates, shown as a percent of maximal WT IL-22 signal. FIG. 3E depicts normalized E max values for phospho-STAT3 and phospho-STAT1 calculated from sigmoidal dose response curves shown in FIG. 3D. In particular, mutation of contact residue Glu117, either alone (E117A, “22-B1”) or in combination with Asn46 (N46A/E117A, “22-B2”), surprisingly resulted in only a slight decrease in STAT3 activation, but an almost complete abrogation of STAT1 signaling (FIGS. 3D-3F). A complex variant including the amino acid substitutions Q116A, K124A, Q128A and S45E (“22-B3”) elicited an even greater level of biased agonism, retaining 100% of the WT IL-22 Eₘₐₓ for STAT3, but only ˜30% for STAT1 (FIGS. 3C-3F). The related IL10Rβ-binding variants comprising Q116A/K124D/Q128A (“22-B4”) and Q48A/Q116A/K124A/Q128A (“22-B5”) also yielded biased agonism, albeit with a greater overall reduction in both STAT1 and STAT3 activation (FIG. 9A). Together, these results show that mutations in IL-22 that partially disrupt the IL10Rβ-binding interface disproportionately weaken STAT1 activation, resulting in STAT3-biased signaling.

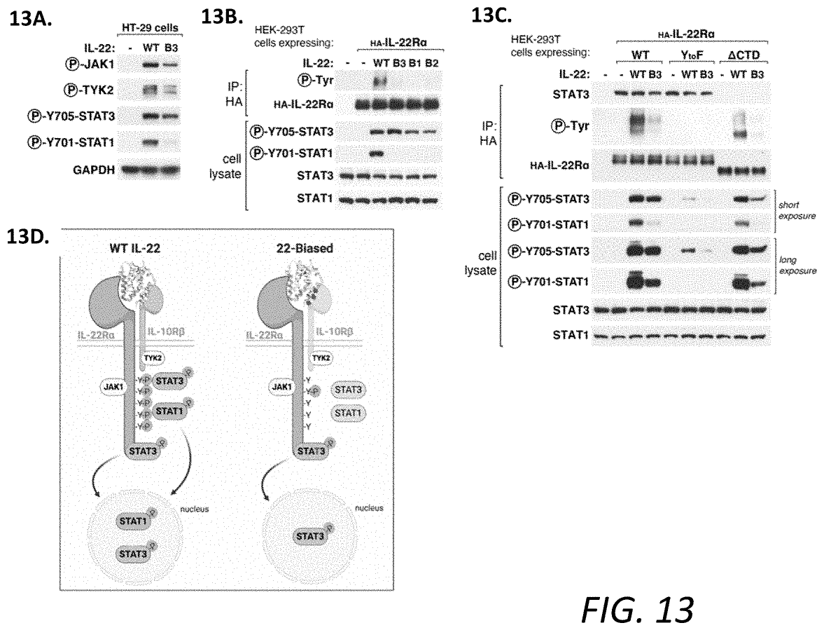

In order to understand the mechanism through which reducing the affinity of IL-22 for IL10Rβ results in biased agonism, the IL-22 receptor signaling in HEK-293T cells was reconstituted by transiently expressing HA-tagged IL22R1 (FIG. 13B). As shown in FIG. 13B, it was observed that biased IL-22 variants induced significantly reduced levels of IL22R1 tyrosine phosphorylation relative to WT IL-22, which correlated with a loss of STAT1 signaling, but had a negligible effect on STAT3 activation. Furthermore, it was observed that in cells expressing an IL22R1 variant lacking all tyrosine residues in the ICD (IL22R1-YtoF), WT IL-22 induced significant activation of STAT3, but not STAT1, thus resembling the signaling profile of the biased agonists on WT IL22R1 (FIG. 13C). This observation is consistent with a previous report showing that a pool of STAT3 is pre-associated with IL22R1 even in the absence of receptor phosphorylation (FIG. 13C), enabling STAT3 activation via both receptor phosphorylation-dependent and independent mechanisms (FIG. 13D). By contrast, STAT1 activation appears to strictly require receptor phosphorylation (FIG. 13B-D). Together, these results support a model in which IL-22 variants with partially defective IL10Rβ-binding affinity induce significantly reduced levels of receptor phosphorylation, which is sufficient to trigger strong activation of STAT3 but not STAT1 due to the two distinct modes of STAT3 activation (FIG. 13D).

### Example 5

### Biased IL-22 Variant 22-B3 Elicits Tissue Selective Signaling

This Example describes experiments performed to demonstrate that an exemplary biased IL-22 variant (22-B3) in accordance with some non-limiting embodiments of the disclosure elicits tissue selective signaling activity.

In these experiments, in order to assess the effects of biasing STAT signaling downstream of the IL-22 receptor in vivo, mice were first administered with a single, high dose injection of WT IL-22 or 22-B3, followed by analysis of STAT3 and STAT1 responses in several IL-22 responsive tissues after 30 minutes (FIG. 4A). It was observed, in the pancreas, the biased IL-22 variant 22-B3 induced full activation of STAT3 relative to WT IL-22, and a strong but slightly reduced activation of STAT1, thereby exhibiting weak STAT3-biased agonism in this tissue (FIG. 4B). In the colon, however, 22-B3 also retained full activation of STAT3, but induced no detectable phosphorylation of STAT1 (FIG. 4C). The biased IL-22 variant 22-B3 therefore functions as a strong biased-agonist in the colon, consistent with the results obtained in HT-29 cells in vitro described above (FIG. 3F). Remarkably, unlike WT IL-22, the biased IL-22 variant 22-B3 elicited no detectable signaling activity in the skin or liver, as assessed by phosphorylation of STAT3 (see, e.g., FIGS. 4D and 4E).

Without being bound to any particular theory, given that the mutations in 22-B3 were designed to weaken the interaction with IL10Rβ, it was hypothesized that differences in IL10Rβ expression could disproportionately affect 22-B3 signaling relative to that of WT IL-22, which explains the observed differences in signaling across tissues. Indeed, RT-qPCR analysis of IL22R1 and IL10Rβ expression in these four mouse tissues revealed that the strength of 22-B3 signaling correlated well with IL10Rβ expression, which is highest in the pancreas, followed by the colon, skin, and then liver (FIG. 4F). A similar pattern of cell type specificity was also observed in human cell lines in vitro, where variants 22-B1, B2 and B3 all retained STAT3 signaling in pancreatic cell-derived PANC-1 cells but elicited no activation of STAT3 or STAT1 in liver-derived HepG2 cells (FIGS. 4G, 4H, and S4A). In particular, it was observed that 22-B3 was also able to block signaling by WT IL-22 in HepG2 cells, demonstrating that 22-B3 retains the ability to bind IL22R1 in these cells and thereby act as an antagonist (FIG. 4I). Thus, IL-22 variant 22-B3 elicits differential signaling behaviors across tissue types in vitro and in vivo, behaving as a weak biased agonist in the pancreas, strong biased agonist colon, and a neutral antagonist in the skin and liver, due to the differential IL10Rβ expression in these tissues.

### Example 6

### 22-B3 Uncouples Tissue Protective and Pro-Inflammatory Functions of IL-22 In Vivo

This Example describes the results from experiments performed to demonstrate that the biased IL-22 variant 22-B3 uncouples expression of tissue protective and pro-inflammatory IL-22 target genes in vivo.

These experiments were designed to address the question as to what extent biasing STAT activation and altering IL-22 tissue specificity affected expression of canonical IL-22 target genes in vivo. In these experiments, RNA was isolated from the pancreas, colon, skin, and liver of mice six hours following a single injection of WT IL-22 or 22-B3 and analyzed the relative expression of known IL-22 target genes by RT-qPCR.

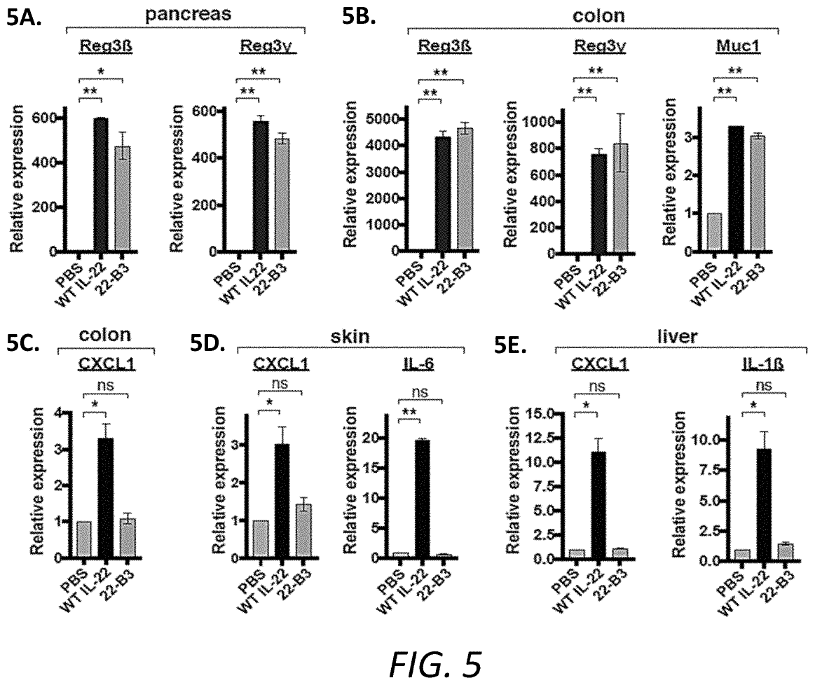

It was observed that in the pancreas, 22-B3 induced significant upregulation of the tissue protective genes encoding regenerating islet-derived proteins Reg3β and Reg3γ, to the same extent as WT IL-22 (FIG. 5A), consistent with the ability of 22-B3 to signal normally through STAT3 in this tissue (FIG. 4B). In the colon, 22-B3 also retained the ability to drive expression of Reg3β, Reg3γ, and Mucinl (FIG. 5B). However, unlike WT IL-22, 22-B3 did not induce colonic expression of the neutrophil-recruiting chemokine CXCL1 (FIG. 5C), a STAT1 target gene that is in part responsible for the pro-inflammatory effects of IL-22 in genetic mouse models of IBD. Furthermore, whereas WT IL-22 increased expression of CXCL1 and the pro-inflammatory cytokine IL-6 in the skin, 22-B3 had no detectable effect on either of these genes (FIG. 5D). Similarly, unlike WT IL-22, 22-B3 did not induce expression of the pro-inflammatory genes CXCL1 and IL-1β in the liver (FIG. 5E). Thus, 22-B3 retains the ability to induce expression of key tissue protective genes in the pancreas and colon but has lost the capacity to induce expression of several pro-inflammatory genes in the colon, skin, and liver.

Additional experiments were performed to test whether the 22-B3-induced changes in gene expression are sufficient to drive the protective effects of IL-22 in a disease setting, where 22-B3 was assayed for its efficacy in a mouse model of acute pancreatitis. In these experiments, mice were pre-treated with two injections of WT IL-22 or 22-B3 at 20 hours and 2 hours before inducing acute pancreatitis via six hourly injections of Caerulein, a cholecystokinin analogue that promotes pancreatic inflammation and elevates the levels of pancreatic enzymes in the blood (see, e.g., FIG. 6A. Importantly, 22-B3 significantly mitigated disease severity to a similar extent as WT IL-22, as measured by the serum levels of pancreatic Lipase and Amylase relative to PBS-treated controls shortly after disease onset (FIG. 6B).

As discussed above, a major potential limitation for the therapeutic use of IL-22 is its ability to increase serum levels of hepatic acute phase-response proteins (APPs) in both mice and humans. In addition to serving as markers of systemic inflammation, APPs such as SAA-1/2 can also contribute to Tₕ17-mediated inflammatory diseases as well. Consistent with previous reports, the experimental data presented herein had demonstrated that just a single injection of WT IL-22 in healthy mice was sufficient to yield a dramatic increase in the serum levels of the APPs SAA-1/2 and Haptoglobin (see, e.g., FIG. 6C). Remarkably, however, administration of 22-B3 had no detectable effect on SAA-1/2 or Haptoglobin levels, even at a high dose (FIG. 6C). Together, these data illustrate that although 22-B3 signaling is sufficient to retain tissue protective functions of IL-22 in vivo, it no longer induces many of the known local and systemic pro-inflammatory effects associated with IL-22.

### Example 7

### Human and Mouse IL-22 Partial Agonists Elicit STAT3 Biased Agonism

This Example describes experiments performed to demonstrate that several mutations in IL-22 at the IL-10Rβ-binding interface result in STAT3-biased signaling.

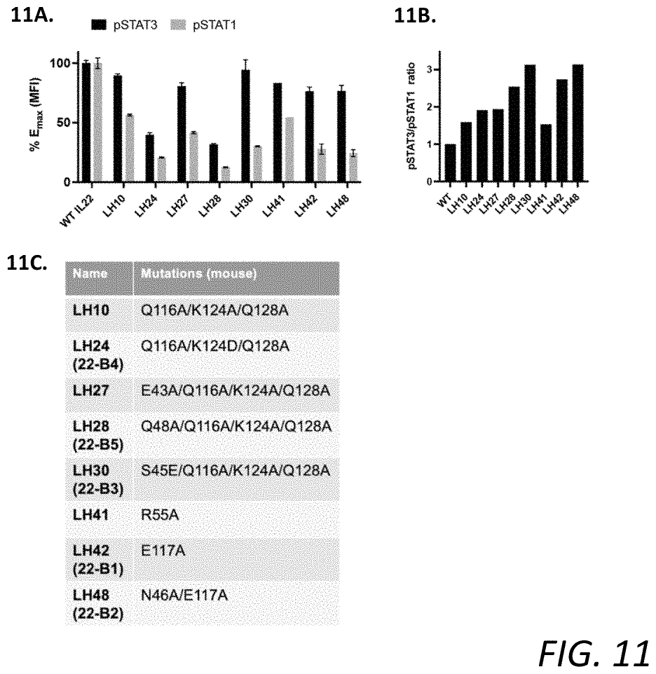

As shown in FIG. 11A, HT-29 (human colorectal carcinoma) cells were treated with varying concentrations of wild-type or engineered mouse IL-22 variants for 20 minutes. Cells were fixed and permeabilized using Methonol/PFA and stained with fluorescently conjugated anti-phospho-STAT1 (AF488) or anti-phospho-STAT3 (AF647) antibodies, and fluorescent intensities were analyzed by flow cytometry. Data were fit to a sigmoidal dose-response curve allowing calculation of the E max for pSTAT1 and pSTAT3 signaling. E max for biased variants were normalized to percentages of wild type IL22. Data shown are mean+/−SEM. FIG. 11B depicts the ratio of the phospho-STAT3/phospho-STAT1 E max for each IL-22 variant, using the data from (A). FIG. 11C is a list of mouse IL-22 variants and corresponding mutations relative to wild-type mouse IL-22.

### Example 8

### Ratio of STAT3 to STAT1 Signaling Elicited by IL-22 Partial Agonists Varies Between IL-22 Partial Agonists

This Example describes experiments performed to illustrate that engineered human IL-22 variants also elicit STAT3-biased signaling in colorectal-derived HT29 cells but are inactive on liver-derived HepG2 cells.

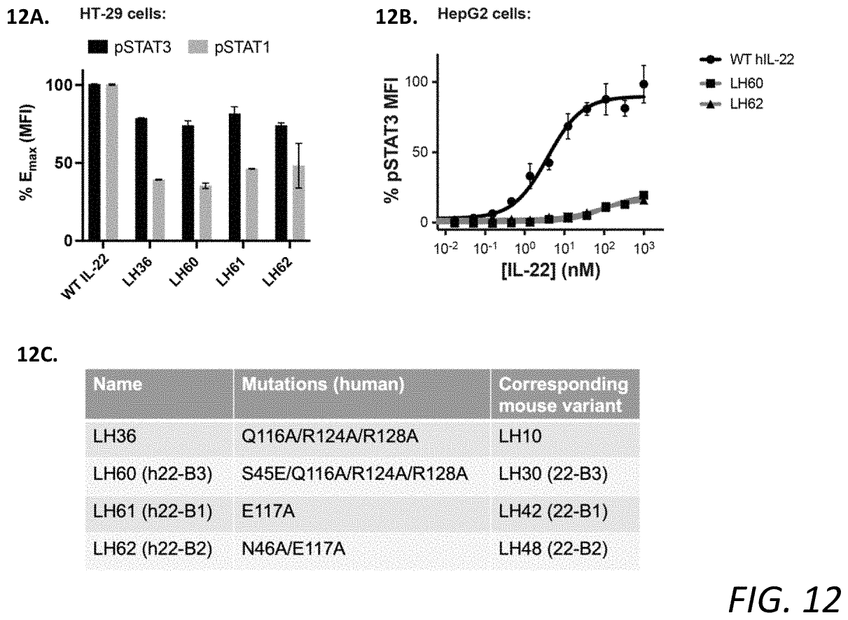

As shown in FIGS. 12A-12B, HT-29 (human colorectal carcinoma; FIG. 12A) or HepG2 (human liver; FIG. 12B) cells were treated with varying concentrations of wild-type or mutant mouse IL-22 for 20 minutes. Cells were fixed and permeabilized using Methonol/PFA and stained with fluorescently conjugated anti-phospho-STAT1 (AF488) or anti-phospho-STAT3 (AF647) antibodies, and fluorescent intensities were analyzed by flow cytometry. Dose-response curves. Data were fit to a sigmoidal dose-response curve allowing calculation of the E max for pSTAT1 and pSTAT3 signaling. E max for biased variants were normalized to percentages of wild type IL22. Data shown are mean+/−SEM for 3 independent replicates. A summary of exemplary human IL-22 variants and corresponding mutations relative to wild-type human IL-22 is provided in FIG. 12C.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

### REFERENCES

Adams, P. D., Afonine, P. V., Bunkoczi, G., Chen, V. B., Davis, I. W., Echols, N., Headd, J. J., Hung, L. W., Kapral, G. J., Grosse-Kunstleve, R. W., et al. (2010). PHENIX: a comprehensive Python-based system for macromolecular structure solution. Acta crystallographica Section D, Biological crystallography 66, 213-221.
Best, M., (2009). Biochemistry 48(28): 6571-6584.
Chao, G., Lau, W. L., Hackel, B. J., Sazinsky, S. L., Lippow, S. M., and Wittrup, K. D. (2006). Isolating and engineering human antibodies using yeast surface display. Nature protocols 1, 755-768.
Dieters, et al., (2004). Site-specific PEGylation of proteins containing unnatural amino acids. Bioorganic and Medicinal Chemistry Letters 14(23):5743-5745.
Dozier and Distefano. Site-Specific PEGylation of Therapeutic Proteins. International Journal of Molecular Science 16(10): 25831-25864, 2015.
Emsley, P., Lohkamp, B., Scott, W. G., and Cowtan, K. (2010). Features and development of Coot. Acta crystallographica Section D, Biological crystallography 66, 486-501.
Evans, P. R. (2011). An introduction to data reduction: space-group determination, scaling and intensity statistics. Acta crystallographica Section D, Biological crystallography 67, 282-292.
Goddard, T. D., Huang, C. C., Meng, E. C., Pettersen, E. F., Couch, G. S., Morris, J. H., and Ferrin, T. E. (2018). UCSF ChimeraX: Meeting modern challenges in visualization and analysis. Protein Science: a publication of the Protein Society 27, 14-25.
Jones, B. C., Logsdon, N. J., and Walter, M. R. (2008). Structure of IL-22 bound to its high-affinity IL-22R1 chain. Structure (London, England: 1993) 16, 1333-1344.
Kabsch, W. (2010). XDS. Acta crystallographica Section D, Biological crystallography 66, 125-132.
Krissinel, E., and Henrick, K. (2007). Inference of macromolecular assemblies from crystalline state. Journal of molecular biology 372, 774-797.
McCoy, A. J., Grosse-Kunstleve, R. W., Adams, P. D., Winn, M. D., Storoni, L. C., and Read, R. J. (2007). Phaser crystallographic software. Journal of applied crystallography 40, 658-674.
Morin, A., Eisenbraun, B., Key, J., Sanschagrin, P. C., Timony, M. A., Ottaviano, M., and Sliz, P. (2013). Collaboration gets the most out of software. eLife 2, e01456.
Oh Jee-Hwan, Schueler L. Kathryn, Donnie S. Stapleton, Laura M. Alexander, Chi-Liang Eric Yen, Mark P. Keller, Alan D. Attie, Jan-Peter van Pijkeren. (2020). Secretion of Recombinant Interleukin-22 by Engineered Lactobacillus reuteri Reduces Fatty Liver Disease in a Mouse Model of Diet-Induced Obesity. mSphere, Vol. 5, Issue 3, May/June 2020.
Lothar Steidler, Sabine Neirynck, Nathalie Huyghebaert, Veerle Snoeck, An Vermeire, Bruno Goddeeris, Eric Cox, Jean Paul Remon and Erik Remaut. (2003). Biological containment of genetically modified Lactococcus lactis for intestinal delivery of human interleukin 10. Nature Biotechnology, Vol. 21, No. 7, July 2003.
Terwilliger, T. C., Grosse-Kunstleve, R. W., Afonine, P. V., Moriarty, N. W., Zwart, P. H., Hung, L. W., Read, R. J., and Adams, P. D. (2008). Iterative model building, structure refinement and density modification with the PHENIX AutoBuild wizard. Acta crystallographica Section D, Biological crystallography 64, 61-69.
Winn, M. D., Ballard, C. C., Cowtan, K. D., Dodson, E. J., Emsley, P., Evans, P. R., Keegan, R. M., Krissinel, E. B., Leslie, A. G., McCoy, A., et al. (2011). Overview of the CCP4 suite and current developments. Acta crystallographica Section D, Biological crystallography 67, 235-242.
Yoon, S. I., Jones, B. C., Logsdon, N. J., Harris, B. D., Deshpande, A., Radaeva, S., Halloran, B. A., Gao, B., and Walter, M. R. (2010). Structure and mechanism of receptor sharing by the IL- 10R2 common chain. Structure (London, England: 1993) 18, 638-648.

What is claimed is:

1. A recombinant polypeptide comprising:
an amino acid sequence having at least 70% sequence identity to an interleukin 22 (IL-22) polypeptide having the amino acid sequence of SEQ ID NO: 1;
and further comprising one or more amino acid substitution at a position corresponding to an amino acid residue selected from the group consisting of X43, X49, X45, X46, X116, X124, and X128 of SEQ ID NO: 1.

2. The recombinant polypeptide of claim 1, wherein the amino acid sequence further comprising an additional amino acid substitution at a position corresponding to an amino acid residue selected from the group consisting of X48, X55, and X117 of SEQ ID NO: 1.

3. The recombinant polypeptide of any one of claims 1 to 2, wherein the one or more amino acid substitution reduces IL10Rβ-binding affinity of the recombinant IL-22 polypeptide compared to a reference IL-22 polypeptide lacking the one or more amino acid substitution.

4. The recombinant polypeptide of any one of claims 1 to 2, wherein the one or more amino acid substitution increases IL10Rβ-binding affinity of the recombinant IL-22 polypeptide compared to a reference IL-22 polypeptide lacking the one or more amino acid substitution.

5. The recombinant polypeptide of any one of claims 1 to 4, wherein the one or more amino acid substitution is at a position corresponding to an amino acid residue selected from the group consisting of X43, X45, X46, X48, X49, X55, and X117 of SEQ ID NO: 1.

6. The recombinant polypeptide of claim 5, further comprising a combination of amino acid substitutions at positions corresponding to amino acid residues X116, X124, X128 of SEQ ID NO: 1.

7. The recombinant polypeptide of any one of claims 1 to 6, wherein the amino acid sequence comprises an amino acid substitution corresponding to amino acid residue X55 or X117 of SEQ ID NO: 1.

8. The recombinant polypeptide of any one of claims 1 to 7, wherein the one or more amino acid substitution is independently selected from the group consisting of an alanine substitution, an arginine substitution, an aspartic acid substitution, a histidine substitution, a glutamic acid substitution, a lysine substitution, a serine substitution, a tryptophan substitution, and combinations of any thereof.

9. The recombinant polypeptide of any one of claims 1 to 8, wherein the one or more amino acid substitution is at a position corresponding to an amino acid residue selected from the group consisting of D43, S45, N46, Q49, Q116, R124, and R128 of SEQ ID NO: 1.

10. The recombinant polypeptide of any one of claims 1 to 9, comprising an amino acid sequence having at least 70% sequence identity to SEQ ID NO: 1, and further comprising the amino acid substitutions corresponding to the following amino acid substitutions:
a) R55A;
b)E117A;
c) N46A/E117A;
d) Q116A/R124A/R128A;
e) Q116A/R124D/R128A;
f) D43A/Q116A/R124A/R128A;
g) S45E/Q116A/R124A/R128A; and
h) Q48A/Q116A/R124A/R128A.

11. The recombinant polypeptide of any one of claims 1 to 9, comprising an amino acid sequence having at least 70% sequence identity to SEQ ID NO: 1, and further comprising an amino acid substitution corresponding an amino acid residue selected from the group consisting of D43H, D43R, S45R, S45G, Q49S, Q49G, Q116W, Q116K, R124Y, and R128K.

12. A recombinant polypeptide comprising:
an amino acid sequence having at least 70% sequence identity to an interleukin 22 (IL-22) polypeptide having the amino acid sequence of SEQ ID NO: 6;
and further comprising one or more amino acid substitution at a position corresponding to an amino acid residue selected from the group consisting of X43, X45, X46, X49, X116, X124, and X128 of SEQ ID NO: 6.

13. The recombinant polypeptide of claim 12, wherein the amino acid sequence further comprising an additional amino acid substitution at a position corresponding to an amino acid residue selected from the group consisting of X48, X55, and X117 of SEQ ID NO: 6.

14. The recombinant polypeptide of any one of claims 12 to 13, wherein the one or more amino acid substitution reduces IL10RB-binding affinity of the recombinant IL-22 polypeptide compared to a reference IL-22 polypeptide lacking the one or more amino acid substitution.

15. The recombinant polypeptide of any one of claims 12 to 13, wherein the one or more amino acid substitution increases IL10Rβ-binding affinity of the recombinant IL-22 polypeptide compared to a reference IL-22 polypeptide lacking the one or more amino acid substitution.

16. The recombinant polypeptide of any one of claims 12 to 15, wherein the one or more amino acid substitution is at a position corresponding to an amino acid residue selected from the group consisting of X43, X45, X46, X48, X55, and X117 of SEQ ID NO: 6.

17. The recombinant polypeptide of claim 16, further comprising a combination of amino acid substitutions at positions corresponding to amino acid residues X116, X124, X128 of SEQ ID NO: 6.

18. The recombinant polypeptide of any one of claims 12 to 17, wherein the amino acid sequence comprises an amino acid substitution corresponding to amino acid residue X55 or X117 of SEQ ID NO: 6.

19. The recombinant polypeptide of any one of claims 12 to 18, wherein the one or more amino acid substitution is independently selected from the group consisting of an alanine substitution, an arginine substitution, an aspartic acid substitution, a histidine substitution, a glutamic acid substitution, a lysine substitution, a serine substitution, a tryptophan substitution, and combinations of any thereof.

20. The recombinant polypeptide of any one of claims 12 to 19, wherein the one or more amino acid substitution is at a position corresponding to an amino acid residue selected from the group consisting of E43, S45, N46, Q48, R55, Q116, E117, K124, Q128 of SEQ ID NO: 6.

21. The recombinant polypeptide of any one of claims 12 to 20, comprising an amino acid sequence having at least 70% sequence identity to SEQ ID NO: 6, and further comprising the amino acid substitutions corresponding to the following amino acid substitutions:
a) R55A;
b)E117A;
c) N46A/E117A;
d) Q116A/K124A/Q128A;
e) Q116A/K124D/Q128A;
f) E43A/Q116A/K124A/Q128A;
g) S45E/Q116A/K124A/Q128A; and
h) Q48A/Q116A/K124A/Q128A.

22. The recombinant polypeptide of any one of claims 12 to 20, comprising an amino acid sequence having at least 70% sequence identity to SEQ ID NO: 6, and further comprising an amino acid substitution corresponding an amino acid residue selected from the group consisting of E43H, E43R, S45R, S45G, Q49S, Q49G, Q116W, Q116K, Q124Y, and Q128K.

23. The recombinant polypeptide of any one of claims 1 to 22, wherein one or more amino acid substitution results in a tissue-selective IL-22 signaling compared to a reference IL-22 polypeptide lacking the one or more amino acid substitution.

24. The recombinant polypeptide of claim 23, wherein the tissue-selective IL-22 signaling comprises a reduction of IL-22 signaling in the skin while substantially retains IL-22 signaling in the pancreas and/or the gastrointestinal tract.

25. The recombinant polypeptide of any one of claims 23 to 24, wherein the tissue-selective IL-22 signaling comprises a reduction of IL-22 signaling in the liver while substantially retains IL-22 signaling in the pancreas and/or the gastrointestinal tract.

26. The recombinant polypeptide of any one of claims 1 to 25, wherein the one or more amino acid substitution results in a biased IL-22 signaling compared to a reference IL-22 polypeptide lacking the one or more amino acid substitution.

27. The recombinant polypeptide of claim 26, wherein the biased IL-22 signaling comprises a reduction in a STAT1-mediated pro-inflammatory function while substantially retains its STAT3-mediated function.

28. The recombinant polypeptide of any one of claims 26 to 27, wherein the biased IL-22 signaling comprises a ratio of STAT1-mediated signaling to STAT3-mediated signaling ranging from 1:1.5 to 1:10.

29. The recombinant polypeptide of claim 28, the STAT1-mediated signaling and/or STAT3-mediated signaling is determined by an assay selected from the group consisting of by a gene expression assay, a phospho-flow signaling assay, and an enzyme-linked immunosorbent assay (ELISA).

30. The recombinant polypeptide of any one of claims 27 to 29, wherein the STAT3-mediated function is selected from the group consisting of tissue protection, tissue regeneration, cell proliferation, and cell survival.

31. The recombinant polypeptide of any one of claims 27 to 30, wherein the STAT1-mediated pro-inflammatory function is selected from the group consisting of cytokine production, chemokine production, and immune cell recruitment.

32. The recombinant polypeptide of any one of claims 27 to 31, wherein the STAT1-mediated pro-inflammatory function is reduced about 20% to about 100%, as determined by a gene expression assay, a phospho-flow signaling assay, and/or an enzyme-linked immunosorbent assay (ELISA).

33. A recombinant nucleic acid molecule comprising a nucleic acid sequence encoding a polypeptide that comprises an amino acid sequence having at least 90% sequence identity to the amino acid sequence of the polypeptide of any one of claims 1 to 32.

34. The nucleic acid molecule of claim 33, wherein the nucleic acid sequence is operably linked to a heterologous nucleic acid sequence.

35. The nucleic acid molecule of any one of claims 33 to 34, wherein the nucleic acid molecule is further defined as an expression cassette or an expression vector.

36. A recombinant cell comprising:
a) a recombinant polypeptide according to any one of claims 1 to 32; and/or
b) a recombinant nucleic acid according to any one of claims 33 to 35.

37. The recombinant cell of claim 36, wherein the recombinant cell is a eukaryotic cell.

38. The recombinant cell of claim 37, wherein the eukaryotic cell is a mammalian cell

39. A cell culture comprising at least one recombinant cell of any one of claims 36 to 38, and a culture medium.

40. A method for producing a polypeptide comprising:
a) providing one or more recombinant cells of any one of claims 1 to 32; and
b) culturing the one or more recombinant cells in a culture medium such that the cells produce the polypeptide encoded by the recombinant nucleic acid molecule.

41. The method of claim 40, further comprising isolating and/or purifying the produced polypeptide.

42. The method of any one of claims 40 to 41, further comprising structurally modifying the produced polypeptide to increase half-life.

43. The method of claim 42, wherein said modification comprises one or more alterations selected from the group consisting of fusion to a human Fc antibody fragment, fusion to albumin, and PEGylation.

44. A recombinant polypeptide produced by the method of any one of claims 40 to 43.

45. A pharmaceutical composition comprising:
a) a recombinant polypeptide according to any one of claims 1-32 and 44;
b) a recombinant nucleic acid according to any one of claims 33 to 35;
c) a recombinant cell according to any one of claims to 36 to 38; and/or
d) a pharmaceutically acceptable carrier.

46. The pharmaceutical composition of claim 45, wherein the composition comprises a recombinant polypeptide according to any one of claims 1-32 and 44, and a pharmaceutically acceptable carrier.

47. The pharmaceutical composition of claim 45, wherein the composition comprises a recombinant nucleic acid according to any one of claims 33 to 35, and a pharmaceutically acceptable carrier.

48. A method for modulating IL-22-mediated signaling in a subject, the method comprising administering to the subject a composition comprising:
a) a recombinant polypeptide according to any one of claims 1-32 and 44;
b) a recombinant nucleic acid according to any one of claims 33 to 35;
c) a recombinant cell according to any one of claims to 36 to 38; and/or
d) a pharmaceutically composition according to claims 45 to 47.

49. A method for the treatment of a condition in a subject in need thereof, the method comprising administering to the subject a composition comprising:
a) a recombinant polypeptide according to any one of claims 1-32 and 44;
b) a recombinant nucleic acid according to any one of claims 33 to 35;
c) a recombinant cell according to any one of claims to 36 to 38; and/or
d) a pharmaceutically composition according to claims 45 to 47.

50. The method of any one of claims 48 to 49, wherein the administered composition results in a tissue-selective IL-22 signaling in the subject compared to a reference IL-22 polypeptide lacking the one or more amino acid substitution.

51. The recombinant polypeptide of claim 50, wherein the tissue-selective IL-22 signaling comprises a reduction of IL-22 signaling in the skin while substantially retains IL-22 signaling in the pancreas and/or the gastrointestinal tract.

52. The recombinant polypeptide of any one of claims 50 to 51, wherein the tissue-selective IL-22 signaling comprises a reduction of IL-22 signaling in the liver while substantially retains IL-22 signaling in the pancreas and/or the gastrointestinal tract.

53. The method of any one of claims 48 to 52, wherein the administered composition results in a biased IL-22 signaling in the subject compared to a reference IL-22 polypeptide lacking the one or more amino acid substitution.

54. The method of claim 53, wherein the biased IL-22 signaling comprises a reduction in a STAT1-mediated pro-inflammatory function while substantially retains its STAT3-mediated function.

55. The method of any one of claims 53 to 54, wherein the biased IL-22 signaling comprises a ratio of STAT1-mediated signaling to STAT3-mediated signaling ranging from 1:1.5 to 1:10.

56. The method of claim 55, wherein the STAT1-mediated signaling and/or STAT3-mediated signaling is determined by an assay selected from the group consisting of by a gene expression assay, a phospho-flow signaling assay, and an enzyme-linked immunosorbent assay (ELISA).

57. The method of any one of claims 54 to 56, wherein the STAT3-mediated function is selected from the group consisting of tissue protection, tissue regeneration, cell proliferation, and cell survival.

58. The method of any one of claims 54 to 57, wherein the STAT1-mediated pro-inflammatory function is selected from the group consisting of cytokine production, chemokine production, and immune cell recruitment.

59. The method of claim 58, wherein the STAT1-mediated pro-inflammatory function is reduced about 20% to about 100%.

60. The method of claim 59, wherein the STAT1-mediated pro-inflammatory function is determined by a gene expression assay, a phospho-flow signaling assay, and/or an enzyme-linked immunosorbent assay (ELISA).

61. The methods of any one of claims 48 to 60, wherein the administered composition results in a reduced capacity to induce expression of a pro-inflammatory gene selected from CXCL1, CXCL2, CXCL8, CXCL9, CXCL10, IL-1β, and IL-6 in the subject.

62. The method of any one of claims 48 to 61, wherein the administered composition substantially retains its capacity to induce expression of a gene selected from Reg3β, Reg3γ, Muc1, Muc2, Muc10, BCL-2, Cyclin-D, Claudin-2, LCN2, and β-Defensin in the subject.

63. The method of any one of claims 48 to 62, wherein the administration of the pharmaceutical composition does not inhibit T-cell activity in the subject.

64. The method of any one of claims 48 to 63, wherein the administered composition enhances epithelial protection and regeneration.

65. The method of any one of claims 48 to 64, wherein the condition is an immune disease, or a chronic infection.

66. The method of claim 65, wherein the immune disease is an autoimmune disease.

67. The method of claim 66, wherein the autoimmune disease is selected from the group consisting of rheumatoid arthritis, insulin-dependent diabetes mellitus, hemolytic anemias, rheumatic fever, thyroiditis, Crohn's disease, myasthenia gravis, glomerulonephritis, autoimmune hepatitis, multiple sclerosis, alopecia areata, psoriasis, vitiligo, dystrophic epidermolysis bullosa, systemic lupus erythematosus, graft vs. host disease, ulcerative colitis, pancreatitis, psoriatic arthritis, and diabetic foot ulcer.

68. The method of claim 66, wherein the autoimmune disease is acute pancreatitis.

69. The method of any one of claims 48 to 68, wherein the subject is a mammal.

70. The method of claim 69, wherein the mammal is a human.

71. The method of any one of claims 48 to 70, wherein the subject has or is suspected of having a condition associated with IL-22 mediated signaling.

72. The method of any one of claims 48 to 71, wherein the composition is administered to the subject individually as a first therapy or in combination with a second therapy.

73. The method of claim 72, wherein the second therapy is selected from the group consisting of chemotherapy, radiotherapy, immunotherapy, hormonal therapy, or toxin therapy.

74. The method of any one of claims 72 to 73, wherein the first therapy and the second therapy are administered concomitantly.

75. The method of any one of claims 72 to 74, wherein the first therapy is administered at the same time as the second therapy.

76. The method of any one of claims 72 to 74, wherein the first therapy and the second therapy are administered sequentially.

77. The method of claim 76, wherein the first therapy is administered before the second therapy.

78. The method of claim 76, wherein the first therapy is administered after the second therapy.

79. The method of claim 76, wherein the first therapy is administered before and/or after the second therapy.

80. The method of any one of claims 72 to 79, wherein the first therapy and the second therapy are administered in rotation.

81. The method of any one of claims 72 to 73, wherein the first therapy and the second therapy are administered together in a single formulation.

82. A kit for modulating IL-22-mediated signaling in a subject, or treating a condition in a subject in need thereof, the kit comprising:
a) a recombinant polypeptide according to any one of claims 1-32 and 44;
b) a recombinant nucleic acid according to any one of claims 33 to 35;
c) a recombinant cell according to any one of claims 36 to 39; and/or
d) a pharmaceutical composition according to any one of claims 45 to 47.
